(19) United States
(12) Patent Application Publication
(10) Pub. No.: US 2015/0211070 A1
(43) Pub. Date: Jul. 30, 2015

(54) COMPOSITIONS AND METHODS FOR ANALYZING HETEROGENEOUS SAMPLES

(71) Applicant: IMMU-METRIX, LLC, Palo Alto, CA (US)

(72) Inventors: Dan Seligson, Palo Alto, CA (US); Thomas Snyder, Palo Alto, CA (US)

(21) Appl. No.: 14/346,293

(22) PCT Filed: Sep. 20, 2012
(86) PCT No.: PCT/US2012/056416

§ 371 Date: Mar. 20, 2014

Related U.S. Application Data
(60) Provisional application No. 61/537,875, filed on Sep. 22, 2011.
(60) Provisional application No. 61/554,086, filed on Nov. 1, 2011.
(60) Provisional application No. 61/608,442, filed on Mar. 8, 2012.

(51) Int. Cl. C12Q 1/68; A61N 5/10; A61K 31/713; C12N 15/113; C07K 16/18; A61K 45/06

(52) U.S. Cl. CPC C12Q 1/6886; C12Q 1/6881; C07K 16/18; A61K 45/06; A61K 31/713; C12N 15/113; A61N 5/1064; C12Q 2600/16; C12Q 2600/156; C12Q 2600/106; C12N 2310/14

(57) ABSTRACT

Methods and compositions for detecting molecules in a heterogeneous sample are disclosed. The methods and compositions disclosed herein may be used for the treatment of a disease or condition characterized by the presence of nucleic acids from at least two different genomic sources. Additionally, the methods and compositions disclosed herein may be used to diagnose, predict, or monitor the status or outcome of a disease or condition characterized by the presence of nucleic acids from at least two different genomic sources. The heterogeneous samples may be from a transplant recipient, a chimeric individual, a subject suffering from a pathogenic infection, or a subject suffering from a different condition such as cancer.

### CROSS-REFERENCE TO RELATED APPLICATIONS

This application claims the benefit under 35 U.S.C. §119(e) of U.S. Provisional Application No. 61/537,875, filed on Sep. 22, 2011; U.S. Provisional Application No. 61/554,086, filed on Nov. 1, 2011; and U.S. Provisional Application No. 61/608,442, filed on Mar. 8, 2012; each of which applications is incorporated herein by reference in its entirety.

### BACKGROUND OF THE INVENTION

Nucleic acids associated with certain pathological or physiological processes are sometimes released into the blood or other bodily fluids of a subject. For example, nucleic acids derived from tumors may be found in bodily fluids of subjects suffering from cancer. Additionally, nucleic acids derived from an unborn fetus may be found in the bodily fluids of pregnant subjects, while nucleic acids derived from donor organs may be found in certain bodily fluids of transplant recipients. As a result, bodily fluids of a subject may contain a heterogeneous mix of nucleic acids from different genomic sources.

Noise or background signal from the genome of a host subject can often make it difficult to detect or distinguish a foreign genome within a biological sample taken from the host. There is thus a need for improved methods for the detection of certain nucleic acids within a heterogeneous sample.

### SUMMARY OF THE INVENTION

Disclosed herein, in some embodiments, is a method comprising (a) obtaining a sample from a subject who is the recipient of transplanted tissue; (b) inserting the sample into a device that generates a size profile of a set of molecules derived from the transplanted tissue; and (c) using the size profile to evaluate the level of necrosis in the transplanted tissue. In some instances, the size profile is generated by paired-end sequencing, single molecule sequencing, gel electrophoresis, capillary electrophoresis, amplification reaction, or arrays. In some instances, the subject is undergoing a rejection of the transplanted tissue. In some instances, the transplanted tissue is a solid organ. In some instances, the method further comprises determining whether the rejection is at least partially caused by an infectious process within the transplanted tissue. In some instances, the method further comprises determining whether the rejection is at least partially caused by an immune reaction to the transplanted tissue. In some instances, the immune reaction is a cell-mediated immune reaction. In some instances, the immune reaction is an antibody-mediated immune reaction. In some instances, the method further comprises comparing the size profile of the set of molecules with the size profile expected if the molecules were derived from necrotic tissue. In some instances, the method further comprises determining the ratio of apoptotic versus necrotic tissue. In some instances, the method further comprises determining the overall levels of a molecule derived from the transplanted tissue. In some instances, the set of molecules are nucleic acids. In some instances, the nucleic acids are DNA molecules. In some instances, the method further comprises conducting a sequencing reaction to detect the sequence of an infectious agent. In some instances, the infectious agent is a virus. In some instances, the infectious agent is a bacterium. In some instances, the method further comprises conducting a sequencing reaction to detect the sequences of molecules from the immune repertoire. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the sample is blood, plasma, a blood fraction, saliva, sputum, urine, semen, transvaginal fluid, cerebrospinal fluid, stool, a cell or a tissue biopsy. In some instances, the sample is blood or plasma. In some instances, the sample is urine.

Further disclosed herein, in some embodiments, is method comprising (a) obtaining a sample of biological fluid from a subject who is the recipient of transplanted tissue; (b) inserting the sample into a device that detects a set of molecules derived from the transplanted tissue; and (c) evaluating the level of necrosis in the transplanted tissue based on the detection of the set of molecules derived from the transplanted tissue. In some instances, the subject is undergoing a rejection of the transplanted tissue. In some instances, the transplanted tissue is a solid organ. In some instances, the method further comprises determining whether the rejection is at least partially caused by an infectious process within the transplanted tissue. In some instances, the method further comprises determining whether the rejection is at least partially caused by an immune reaction to the transplanted tissue. In some instances, the immune reaction is a cell-mediated immune reaction. In some instances, the immune reaction is an antibody-mediated immune reaction. In some instances, the method further comprises determining the overall levels of a molecule derived from the transplanted tissue. In some instances, the set of molecules are nucleic acids. In some instances, the nucleic acids are DNA molecules. In some instances, the method further comprises conducting a sequencing reaction to detect the sequence of an infectious agent. In some instances, the infectious agent is a virus. In some instances, the infectious agent is a bacterium. In some instances, the method further comprises conducting a sequencing reaction to detect the sequences of molecules from the immune repertoire. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the sample is blood, plasma, a blood fraction, saliva, sputum, urine, semen, transvaginal fluid, cerebrospinal fluid, stool, a cell or a tissue biopsy. In some instances, the sample is blood or plasma. In some instances, the sample is urine.

Disclosed herein, in some embodiments, is a method comprising: (a) obtaining a sample from a subject who is the recipient of transplanted tissue; (b) inserting the sample into a device that generates a size profile of a set of molecules derived from the transplanted tissue; and (c) using the size profile to evaluate the level of apoptosis in the transplanted tissue in order to detect or evaluate the risk of rejection of the transplanted tissue. In some instances, the subject is undergoing a rejection of the transplanted tissue. In some instances, the transplanted tissue is a solid organ. In some instances, the method further comprises determining whether the rejection is at least partially caused by an infectious process within the transplanted tissue. In some instances, the method further comprises determining whether the rejection is at least partially caused by an immune reaction to the transplanted tissue. In some instances, the immune reaction is a cell-mediated immune reaction. In some instances, the immune reaction is an antibody-mediated immune reaction. In some instances, the method further comprises comparing the size profile of the set of molecules with the size profile expected if the molecules were derived from apoptotic tissue. In some instances, the method further comprises determining the ratio of apoptotic versus necrotic tissue. In some instances, the method further comprises determining the overall levels of a molecule derived from the transplanted tissue. In some instances, the set of molecules are nucleic acids. In some instances, the nucleic acids are DNA molecules. In some instances, the method further comprises conducting a sequencing reaction to detect the sequence of an infectious agent. In some instances, the infectious agent is a virus. In some instances, the infectious agent is a bacterium. In some instances, the method further comprises conducting a sequencing reaction to detect the sequences of molecules from the immune repertoire. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the sample is blood, plasma, a blood fraction, saliva, sputum, urine, semen, transvaginal fluid, cerebrospinal fluid, stool, a cell or a tissue biopsy. In some instances, the sample is blood or plasma. In some instances, the sample is urine.

Further disclosed herein, in some embodiments, is a method of differential detection of whole genomes, or unique regions thereof, in a biological sample comprising a mixture of genetic material from different genomic sources, the method comprising the steps of: (a) isolating nucleic acid from the biological sample comprising a mixture of genetic material from different genomic sources to obtain a heterogeneous nucleic acid sample; (b) directly sequencing the heterogeneous nucleic acid sample without diluting or distributing the sample into discrete sub-samples or individual molecules; (c) counting the number of unique sequences in the heterogeneous nucleic acid sample; and (d) conducting an analysis that compares the ratios of unique sequences to determine the relative amounts of the different genomes in the biological sample. In some instances, the unique region of the genome comprises one or more variable number tandem repeats (VNTRs), short tandem repeat (STRs), SNP patterns, hypervariable regions, minisatellites, dinucleotide repeats, trinucleotide repeats, tetranucleotide repeats, and simple sequence repeats. In some instances, the sequencing step is performed using long-read sequencing technology. In some instances, the long-read sequencing technology is selected from the group consisting of: the SMRT™ sequencing system, the SOLiD™ sequencing system, the SOLEXA™ sequencing system, the Ion Torrent™ sequencing system, or the Genome Sequencer FLX system. In some instances, the biological sample is blood, a blood fraction, saliva, sputum, urine, semen, transvaginal fluid, cerebrospinal fluid, stool, a cell or a tissue biopsy. In some instances, the blood is peripheral blood derived from a subject diagnosed with or suspected of having cancer, or a fraction thereof. In some instances, the blood is peripheral blood derived from a transplant recipient, or a fraction thereof. In some instances, the different genomic sources are selected from the group consisting of: a pregnant female and a fetus, an organ donor and a transplant recipient, cancerous cell and a non-cancerous cell. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the cancer is prostate, breast, ovarian, lung, colon, pancreatic, or skin cancer.

Disclosed herein, in some embodiments, is a method of treating a subject, the method comprising the steps of: (a) administering a therapeutic regimen to the subject; (b) obtaining a biological sample from the subject and detecting a quantity of nucleic acid from at least one different genomic source within the sample, wherein the at least one different genomic source is different from the subject; and (c) adjusting the therapeutic regimen administered to the subject based on the amount of nucleic acids from the at least one different genomic source, wherein the therapeutic regimen is increased if the percentage of nucleic acids from the at least one different genomic source is greater than 0.5% of the total nucleic acids in the biological sample. In some instances, the percentage of nucleic acids from the at least one different genomic source is less than 1.5% of the total nucleic acids in the biological sample. In some instances, the percentage of nucleic acids from the at least one different genomic source is greater than 1% of the total nucleic acids in the biological sample. In some instances, the subject is a recipient of a heart transplant. In some instances, the biological sample is blood, a blood fraction, saliva, sputum, urine, semen, transvaginal fluid, cerebrospinal fluid, stool, a cell or a tissue biopsy. In some instances, the biological sample is blood. In some instances, the blood is peripheral blood derived from a transplant recipient, or a fraction thereof. In some instances, the blood is peripheral blood derived from a subject diagnosed with or suspected of having cancer, or a fraction thereof. In some instances, the subject is a transplant recipient or afflicted with cancer. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the cancer is prostate, breast, ovarian, lung, colon, pancreatic, or skin cancer. In some instances, the therapeutic regimen is an immune suppression regimen. In some instances, the therapeutic regimen is reduced by at least 50%. In some instances, the immune suppression regimen comprises administering to the subject a glucocorticoid, a cytostatic agent, an anti-metabolite, an antibody, or drugs acting on immunophilins to the subject. In some instances, the immune suppression regimen comprises administering to the subject an anti-IL2 antibody. In some instances, the immune suppression regimen comprises administering to the subject cyclophilin, mycophenolate, basiliximab, daclizumab, tacrolimus, sirolimus, sacrolimus, interferon, opioid, TNF-α (tumor necrosis factor-alpha) binding protein, fingolimod or myriocin. In some instances, the immune suppression regimen comprises administering to the subject CellCept, ProGraf, Simulect, Zenapax, Rapamune, or Nulojix. In some instances, the therapeutic regimen is a chemotherapeutic regimen, a radiation therapy regimen, a monoclonal antibody regimen, an anti-angiogenic regimen, an oligonucleotide therapeutic regimen, or any combination thereof. In some instances, the oligonucleotide therapeutic regimen comprises the administration of an antisense oligonucleotide, miRNA, siRNA, aptamer, or RNA-based therapeutic to the subject. In some instances, the method further comprises sequencing the nucleic acids. In some instances, the sequencing is performed using long-read sequencing technology. In some instances, the long-read sequencing technology is selected from the group consisting of: the SMRT™ sequencing system, the SOLiD™ sequencing system, the SOLEXA™ sequencing system, the Ion Torrent™ sequencing system, or the Genome Sequencer FLX system. In some instances, the method further comprises the steps of counting the number of unique sequences of nucleic acids; and conducting an analysis that compares the ratios of unique sequences to determine the relative amounts of the different genomes in the biological sample. In some instances, the unique region of the genome comprises one or more variable number tandem repeats (VNTRs), short tandem repeat (STRs), SNP patterns, hypervariable regions, minisatellites, dinucleotide repeats, trinucleotide repeats, tetranucleotide repeats, and simple sequence repeats.

Further disclosed herein, in some embodiments, is a method of treating a subject, the method comprising the steps of: (a) administering a therapeutic regimen to the subject; (b) at a first point of time, obtaining a first biological sample from the subject; (c) detecting a first quantity of nucleic acids from at least one different genomic source within the first biological sample, wherein the at least one different genomic source is different from the subject; (d) at a second point of time, obtaining a second biological sample from the subject at a point of time wherein a transplant rejection is detectable by a biopsy and wherein the second point of time is within a three-month period after the obtaining of the first biological sample from the subject; (e) detecting a second quantity of the nucleic acids from the at least one genomic source within the second biological sample; and (f) adjusting the therapeutic regimen administered to the subject based on the first and second quantities, wherein the therapeutic regimen is increased if the second quantity of nucleic acids is greater than 2.5-fold higher than the first quantity of nucleic acids. In some instances, the biological sample is blood, a blood fraction, saliva, sputum, urine, semen, transvaginal fluid, cerebrospinal fluid, stool, a cell or a tissue biopsy. In some instances, the biological sample is blood. In some instances, the blood is peripheral blood derived from a transplant recipient, or a fraction thereof. In some instances, the blood is peripheral blood derived from a subject diagnosed with or suspected of having cancer, or a fraction thereof. In some instances, the subject is a transplant recipient or afflicted with cancer. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the cancer is prostate, breast, ovarian, lung, colon, pancreatic, or skin cancer. In some instances, the therapeutic regimen is an immune suppression regimen. In some instances, the therapeutic regimen is reduced by at least 50%. In some instances, the immune suppression regimen comprises administering to the subject a glucocorticoid, a cytostatic agent, an anti-metabolite, an antibody, or drugs acting on immunophilins to the subject. In some instances, the immune suppression regimen comprises administering to the subject an anti-IL2 antibody. In some instances, the immune suppression regimen comprises administering to the subject cyclophilin, mycophenolate, basiliximab, daclizumab, tacrolimus, sirolimus, sacrolimus, interferon, opioid, TNF-α (tumor necrosis factor-alpha) binding protein, fingolimod or myriocin. In some instances, the immune suppression regimen comprises administering to the subject CellCept, ProGraf, Simulect, Zenapax, Rapamune, or Nulojix. In some instances, the therapeutic regimen is a chemotherapeutic regimen, a radiation therapy regimen, a monoclonal antibody regimen, an anti-angiogenic regimen, an oligonucleotide therapeutic regimen, or any combination thereof. In some instances, the oligonucleotide therapeutic regimen comprises the administration of an antisense oligonucleotide, miRNA, siRNA, aptamer, or RNA-based therapeutic to the subject. In some instances, the method further comprises sequencing the nucleic acids. In some instances, the sequencing is performed using long-read sequencing technology. In some instances, the long-read sequencing technology is selected from the group consisting of: the SMRT™ sequencing system, the SOLiD™ sequencing system, the SOLEXA™ sequencing system, the Ion Torrent™ sequencing system, or the Genome Sequencer FLX system. In some instances, the method further comprises the steps of counting the number of unique sequences of nucleic acids; and conducting an analysis that compares the ratios of unique sequences to determine the relative amounts of the different genomes in the biological sample. In some instances, the unique region of the genome comprises one or more variable number tandem repeats (VNTRs), short tandem repeat (STRs), SNP patterns, hypervariable regions, minisatellites, dinucleotide repeats, trinucleotide repeats, tetranucleotide repeats, and simple sequence repeats.

Further disclosed herein, in some embodiments, is a method of treating a subject, the method comprising the steps of (a) administering a therapeutic regimen to the subject; (b) isolating nucleic acid from a biological sample obtained from the subject and detecting within the biological sample a quantity of nucleic acids from at least one different genomic source, wherein the at least one different genomic source is different from the subject; and (c) adjusting the therapeutic regimen administered to the subject based on the amount of nucleic acids from different genomic sources detected in said biological sample, wherein the therapeutic regimen is reduced or stopped if the percentage of nucleic acids from the at least one different genomic source is less than 1% of the total nucleic acids in said biological sample. In some instances, the subject is a recipient of a lung transplant, a kidney transplant or a liver transplant. In some instances, the percentage of nucleic acids from the at least one different genomic source is less than 0.5% of the total nucleic acids in said biological sample. In some instances, the biological sample is blood, a blood fraction, saliva, sputum, urine, semen, transvaginal fluid, cerebrospinal fluid, stool, a cell or a tissue biopsy. In some instances, the biological sample is blood. In some instances, the blood is peripheral blood derived from a transplant recipient, or a fraction thereof. In some instances, the blood is peripheral blood derived from a subject diagnosed with or suspected of having cancer, or a fraction thereof. In some instances, the subject is a transplant recipient or afflicted with cancer. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the cancer is prostate, breast, ovarian, lung, colon, pancreatic, or skin cancer. In some instances, the therapeutic regimen is an immune suppression regimen. In some instances, the therapeutic regimen is reduced by at least 50%. In some instances, the immune suppression regimen comprises administering to the subject a glucocorticoid, a cytostatic agent, an anti-metabolite, an antibody, or drugs acting on immunophilins to the subject. In some instances, the immune suppression regimen comprises administering to the subject an anti-IL2 antibody. In some instances, the immune suppression regimen comprises administering to the subject cyclophilin, mycophenolate, basiliximab, daclizumab, tacrolimus, sirolimus, sacrolimus, interferon, opioid, TNF-α (tumor necrosis factor-alpha) binding protein, fingolimod or myriocin. In some instances, the immune suppression regimen comprises administering to the subject CellCept, ProGraf, Simulect, Zenapax, Rapamune, or Nulojix. In some instances, the therapeutic regimen is a chemotherapeutic regimen, a radiation therapy regimen, a monoclonal antibody regimen, an anti-angiogenic regimen, an oligonucleotide therapeutic regimen, or any combination thereof. In some instances, the oligonucleotide therapeutic regimen comprises the administration of an antisense oligonucleotide, miRNA, siRNA, aptamer, or RNA-based therapeutic to the subject. In some instances, the method further comprises sequencing the nucleic acids. In some instances, the sequencing is performed using long-read sequencing technology. In some instances, the long-read sequencing technology is selected from the group consisting of: the SMRT™ sequencing system, the SOLiD™ sequencing system, the SOLEXA™ sequencing system, the Ion Torrent™ sequencing system, or the Genome Sequencer FLX system.

Further disclosed herein, in some embodiments, is a method of monitoring the immune system in a subject, said method comprising the steps of (a) administering a therapeutic regimen to the subject; (b) at a first point of time, obtaining a first biological sample from the subject and detecting a first quantity of nucleic acid from at least one different genomic source within the biological sample, wherein the at least one different genomic source is different from the subject; (c) at a second point of time, obtaining a second biological sample from the subject at a point of time within three months after the first point of time; (d) detecting a second quantity of nucleic acids from the at least one genomic source within the second biological sample; and (e) adjusting the therapeutic regimen administered to the subject based on the first and second quantities of nucleic acids, wherein the therapeutic regimen is increased if the second quantity of nucleic acids is greater than five-fold higher than the first quantity of nucleic acids. In some instances, the biological sample is blood, a blood fraction, saliva, sputum, urine, semen, transvaginal fluid, cerebrospinal fluid, stool, a cell or a tissue biopsy. In some instances, the biological sample is blood. In some instances, the blood is peripheral blood derived from a transplant recipient, or a fraction thereof. In some instances, the blood is peripheral blood derived from a subject diagnosed with or suspected of having cancer, or a fraction thereof. In some instances, the subject is a transplant recipient or afflicted with cancer. In some instances, the transplant recipient has received a kidney transplant, a pancreas transplant, a liver transplant, a heart transplant, a lung transplant, an intestine transplant, a pancreas after kidney transplant, or a simultaneous pancreas-kidney transplant from the organ donor. In some instances, the cancer is prostate, breast, ovarian, lung, colon, pancreatic, or skin cancer. In some instances, the therapeutic regimen is an immune suppression regimen. In some instances, the therapeutic regimen is reduced by at least 50%. In some instances, the immune suppression regimen comprises administering to the subject a glucocorticoid, a cytostatic agent, an anti-metabolite, an antibody, or drugs acting on immunophilins to the subject. In some instances, the immune suppression regimen comprises administering to the subject an anti-IL2 antibody. In some instances, the immune suppression regimen comprises administering to the subject cyclophilin, mycophenolate, basiliximab, daclizumab, tacrolimus, sirolimus, sacrolimus, interferon, opioid, TNF-α (tumor necrosis factor-alpha) binding protein, fingolimod or myriocin. In some instances, the immune suppression regimen comprises administering to the subject CellCept, ProGraf, Simulect, Zenapax, Rapamune, or Nulojix. In some instances, the therapeutic regimen is a chemotherapeutic regimen, a radiation therapy regimen, a monoclonal antibody regimen, an anti-angiogenic regimen, an oligonucleotide therapeutic regimen, or any combination thereof. In some instances, the oligonucleotide therapeutic regimen comprises the administration of an antisense oligonucleotide, miRNA, siRNA, aptamer, or RNA-based therapeutic to the subject. In some instances, the method further comprises sequencing the nucleic acids. In some instances, the sequencing is performed using long-read sequencing technology. In some instances, the long-read sequencing technology is selected from the group consisting of: the SMRT™ sequencing system, the SOLiD™ sequencing system, the SOLEXA™ sequencing system, the Ion Torrent™ sequencing system, or the Genome Sequencer FLX system.

Further disclosed herein, in some embodiments, is a method of treating a subject who has received a lung transplant from a donor comprising the steps of (a) providing a biological sample from the subject; (b) detecting within the biological sample a quantity of nucleic acids derived from the donor; and (c) administering a therapeutic regimen to the subject wherein at least 1% of the total nucleic acids in the biological sample comprise the donor nucleic acids. In some instances, at least 3% of the total nucleic acids in the biological sample comprise the donor nucleic acids.

Further disclosed herein, in some embodiments, is a method of treating a subject who has received a lung transplant from a donor comprising the steps of (a) administering a therapeutic regimen to the subject; (b) obtaining a biological sample from the subject from at last two different time points; (c) determining a quantity of nucleic acids derived from the donor at the at least two different time points; and (d) reducing or stopping the therapeutic regimen when the percentage of the total nucleic acids in the sample comprising the donor nucleic acids is less than 1.5%. In some instances, the percentage of the total nucleic acids in the sample comprising donor nucleic acids is less than 0.5%.

Further disclosed herein, in some embodiments, is a method of treating a subject who has received a liver transplant from a donor comprising the steps of (a) providing a biological sample from the subject; (b) detecting within the biological sample a quantity of nucleic acids derived from the donor; and (c) administering a therapeutic regimen to the subject wherein at least 1.5% of the total nucleic acids in the biological sample comprise the donor nucleic acids. In some instances, at least 4% of the total nucleic acids in the biological sample comprise the donor nucleic acids.

Further disclosed herein, in some embodiments, is a method of treating a subject who has received a liver transplant from a donor comprising the steps of (a) administering a therapeutic regimen to the subject; (b) obtaining a biological sample from the subject from at last two different time points; (c) determining a quantity of nucleic acids derived from the donor at the at least two different time points; and (d) reducing or stopping the therapeutic regimen when the percentage of the total nucleic acids in the sample comprising the donor nucleic acids is less than 2%. In some instances, the percentage of the total nucleic acids in the sample comprising the donor nucleic acids is less than 0.75%.

Further disclosed herein, in some embodiments, is a method of treating a subject who has received a transplant from a donor comprising the steps of: (a) providing a biological sample from the subject; (b) detecting within the biological sample a quantity of nucleic acids derived from the donor; and (c) administering a therapeutic regimen to the subject when the quantity of donor nucleic acids increases by greater than 2.5 fold over at least a one-month period. In some instances, the 2.5-fold increase is predictive that a transplant rejection will occur within at least one month. In some instances, the 2.5-fold increase is predictive that a transplant rejection will occur within at least three months.

Also disclosed herein, in some embodiments, is a method comprising (a) obtaining a sample from a subject who is the recipient of a transplanted tissue; (b) conducting a reaction on the sample to detect a molecule from a pathogen, wherein the reaction comprises a sequencing reaction; and (c) diagnosing, predicting, or monitoring a status or outcome of a condition in the subject based on the detection of the molecule from a pathogen. In some instances, the pathogen is a virus. In some instances, the pathogen is a bacterium. In some instances, the pathogen is derived from the transplanted tissue. In some instances, the pathogen is introduced as a result of the subject receiving the transplanted tissue. In some instances, the sample is a biological fluid. In some instances, the biological fluid is blood or plasma. In some instances, the biological fluid is urine.

Also disclosed herein, in some embodiments, is a method comprising (a) obtaining a sample from a subject who is the recipient of a transplanted tissue; (b) conducting a reaction on the sample to detect a molecule from a pathogen, wherein the reaction comprises attaching one or more unique identifiers to the molecule from a pathogen; and (c) diagnosing, predicting, or monitoring a status or outcome of a condition in the subject based on the detection of the molecule from a pathogen. In some instances, the pathogen is a virus. In some instances, the pathogen is a bacterium. In some instances, the unique identifiers comprise nucleic acids. In some instances, the reaction is a sequencing reaction. In some instances, the pathogen is derived from the transplanted tissue. In some instances, the pathogen is introduced as a result of the subject receiving the transplanted tissue. In some instances, the sample is a biological fluid. In some instances, the biological fluid is blood or plasma. In some instances, the biological fluid is urine.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative instances, in which the principles of the invention are utilized, and the accompanying drawings of which:

FIG. 1 Illustration of a workflow for detecting molecules within a heterogeneous sample

FIG. 2 Illustration of a workflow for detecting foreign molecules

### DETAILED DESCRIPTION OF THE INVENTION

### I. Overview

This disclosure provides methods, compositions, and systems for detecting molecules (e.g., nucleic acids, proteins, etc.) in a heterogeneous sample, such as a sample comprising nucleic acids derived from at least two different genomic sources. The heterogeneous sample may be a biological sample obtained from a subject and may comprise both the subject's molecules and foreign molecules (e.g., nucleic acids, proteins, etc.) that originated from donor tissue, a pathogen, a fetus, or other source. However, in some cases, the so-called foreign molecules are derived from the subject's own tissue that has transformed in some way—such as by becoming cancerous, or experiencing cellular death (e.g., by necrosis or apoptosis). In such cases, the heterogeneous sample may comprise molecules derived from the subject's healthy tissue as well as molecules derived from tissue that has undergone such change or transformation.

This disclosure is particularly useful for the differential diagnosis of a condition. For example, often the origin of a graft injury experienced by an organ transplant recipient is difficult to determine A graft injury can also be caused by more than one factor. The present disclosure can enable detection, approximation, or identification of the cause (or multiple causes) of the injury. It also discloses methods of distinguishing between different causes. There are many potential causes of a graft injury including, but not limited to: (1) an immune-mediated rejection of the transplanted tissue and (2) a pathogenic infection. This disclosure provides methods of evaluating a heterogeneous sample in order to evaluate the level of necrosis or apoptosis in the transplanted tissue (or surrounding tissue). The relative levels of necrosis and apoptosis can then be used to assess whether the injury is due to a pathogenic infection (which may correlate with higher levels of necrotic tissue) or a cellular immune response (which may correlate with higher levels of apoptotic tissue). The present disclosure also provides therapeutic regimens, diagnostics, prognostics, and methods of monitoring a condition.

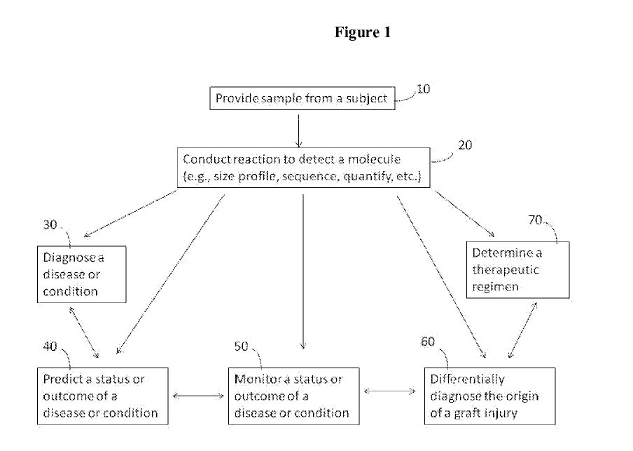

FIG. 1 provides a general overview of the flow of many of the methods provided herein. Generally, the method comprises providing a sample from a subject (10), conducting a reaction to detect a molecule (20), and then diagnosing a disease or condition (30), predicting the status or outcome of a disease or condition (40), monitoring the status or outcome of a disease or condition (50), differentially diagnosing the origin of a graft injury (60), or determining a therapeutic regimen (70). Different combinations of steps can be used, and the steps can be performed in different orders as well. Also provided are methods for detecting, monitoring, and/or measuring whole genomes, or unique regions thereof, within the heterogeneous sample. The genomes (or genotypic patterns) may derive from a subject or from a foreign source.

In some instances, the methods further comprise the use of a computer, computer software, and/or algorithm for analyzing one or more molecules in the sample. In other instances, the methods further comprise generating a report.

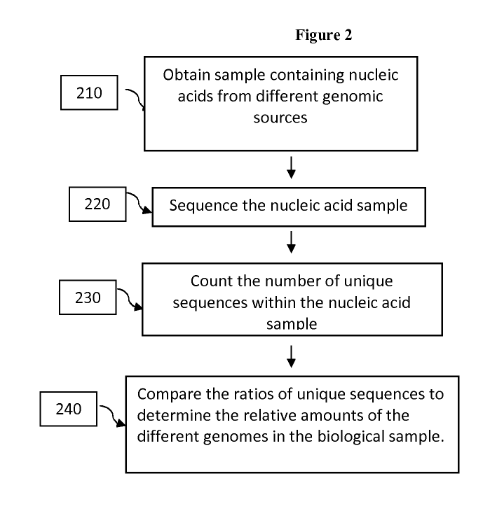

FIG. 2 outlines some additional embodiments of the methods provided herein. The methods may generally comprise: (a) obtaining a sample containing nucleic acids from different genomic sources (110); (b) optionally, sequencing the nucleic acids, e.g., by long-read sequencing (120); (c) optionally, counting the number of unique sequences within the nucleic acid sample (e.g., via sequence reads) (130); and (d) optionally, analyzing (e.g., comparing) the ratios of unique sequences to determine the relative amounts of the different genomes in the biological sample (140). Different combinations of steps can be used, and the steps can be performed in different orders as well, or combined with steps described herein related to other methods.

The methods, compositions, and systems of the disclosure may be especially useful for noninvasive detection of organ rejection in a transplant recipient, cancer in a subject, fetal genetic disorders in a fetus (via the maternal blood), and infection by foreign pathogens. The methods provided herein are also useful for the detection of single nucleotide polymorphisms (SNPs), as well as the detection of any genomic instability, such as a point mutation or an aneuploidy (e.g., trisomy, monosomy, duplication, deletion, addition, rearrangement, translocation, or inversion) within a foreign and/or host genome.

### II. Organ/Tissue Transplantation

Introduction

This disclosure provides methods for detecting circulating molecules (e.g., nucleic acids, proteins, etc.) in a subject who has received a transplant (e.g., organ transplant, tissue transplant, stem cell transplant) in order to diagnose, monitor, predict, or evaluate the status or outcome of the transplant. Moreover, this disclosure provides methods of determining or evaluating potential causes of transplant rejection, or threatened-rejection.

Often, a biological sample containing blood (or other bodily fluid such as urine) obtained from a transplant recipient is a heterogeneous sample containing molecules derived both from the donor and the recipient. The method may comprise specifically detecting, profiling, or quantitating molecules (e.g., nucleic acids, DNA, RNA, protein, etc.) that are within the biological sample and that derive from the donor or donor tissue. In some cases, the method comprises detecting nucleic acids (or other molecules) that are derived from the transplant recipient's tissue (as opposed to the donor tissue)—either alone or in addition to molecules derived from donor tissue.

A relative rise in the level of certain circulating nucleic acids, particularly those derived from the donor organ or tissue, generally indicates an increased risk of rejection—or actual rejection—of the transplanted tissue. Since cell-free DNA or RNA can arise from dying cells (e.g., apoptotic cells or necrotic cells), the relative amount of donor-specific sequences in circulating nucleic acids may provide a predictive measure of on-coming organ failure in transplant patients for many types of solid organ transplantation including, but not limited to, heart, lung, liver, kidney and skin. Thus, transplant rejection can be detected or predicted using partial or whole genome analysis of circulating nucleic acids derived from the donor as compared to the recipient's genome.

a. Differential Diagnosis of Graft Injuries

i. Types of Tissue Transplant Outcomes (or Statuses)

A subject who has received a tissue or organ transplant has a number of different possible outcomes. Under optimal circumstances, the status or outcome of the tissue transplant is transplant tolerance. Transplant tolerance includes situations where the subject does not reject a graft organ, tissue or cell(s) that has been introduced into/onto the subject. In other words, the subject tolerates or maintains the organ, tissue or cell(s) that has been transplanted to it.

Less-favorable statuses or outcomes may involve immunological rejection (e.g., acute cellular rejection, antibody-mediated rejection) of the transplant, transplant (or graft) injury (either non-rejection-based, or due to rejection), decreased or impaired transplant function, decreased transplant survival, and/or chronic transplant injury. Even worse statuses or outcomes include organ failure and death of the organism. Organ failure can involve failure of the whole organ, or a portion thereof. Organ failure may also involve one organ, or multiple organs, e.g., greater than 1, 2, 3, 4, 5, 6, 7, 8, 9, or 10 organs.

Transplant rejection encompasses both acute and chronic transplant rejection. Acute rejection (AR) may occur when the immune system of a tissue transplant recipient rejects transplanted tissue, usually because it is immunologically foreign. Acute rejection may be characterized by infiltration of the transplanted tissue by immune cells of the recipient, which carry out their effector function and destroy the transplanted tissue. The onset of acute rejection may be rapid and generally occurs in humans within a few weeks or a few months after transplant surgery, but in some cases acute rejection may occur several months after transplant surgery or even years after transplant surgery. Generally, acute rejection can be inhibited or suppressed with immunosuppressive drugs such as rapamycin, cyclosporin A, anti-CD4OL monoclonal antibody and the like. Chronic transplant rejection (CR) generally occurs in humans within several months to years after engraftment, and can occur even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include cardiac allograft vasculopathy and fibrotic atherosclerosis; in kidney transplants, such disorders include obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs. In some instances, chronic rejection comprises inflammation at the site of a graft and/or surrounding vasculature. In some instances, chronic rejection comprises injury to a graft and/or surrounding vasculature. Chronic rejection can be caused by a pathogenic infection (e.g., viral, bacterial, fungal, microbial). For example, a viral infection can cause a chronic rejection. Alternatively, a bacterial infection can cause a chronic rejection. Chronic rejection can be characterized by a slow accumulation of injury. Chronic rejection can occur over a prolonged period of time, such as over several weeks (e.g., about 2 weeks, about 4 weeks, about 6 weeks, about 8 weeks, about 10 weeks, about 12 weeks). In some instances, chronic rejection occurs over several months (e.g., about 3 months, about 6 months, about 9 months, about 12 months). Chronic rejection can occur over several years (e.g., about 1.5 years, about 2 years, about 2.5 years, about 3 years, about 3.5 years, about 4 years, about 4.5 years, about 5 years). In some instances, the immune activity of a transplant recipient continues, extends, or prolongs the duration of the chronic rejection.

Examples of non-rejection based transplant injury (e.g., allograft injury) include, but are not limited to, ischemic injury, pathogenic infection (e.g., viral infection, bacterial infection, fungal infection), perioperative ischemia, reperfusion injury, hypertension, physiological stress, injuries due to reactive oxygen species and injuries caused by pharmaceutical agents (e.g., immunosuppressive drugs, etc.). Transplant status or outcome may also involve vascular complications or neoplastic involvement of the transplanted organ. The outcome or status of a transplant can be affected by the dose, titer or level of therapies used to treat the subject, such as the level of immunosuppressive agents administered to the subject. For example, a high dose of immunosuppressive drugs may result in transplant injury, while a dose that is too low may result in rejection of the transplant.

ii. Circulating Donor Molecules and Cellular Death by Apoptosis or Necrosis

The present disclosure provides methods for identifying a source or cause of a transplant/graft injury or of transplant rejection, including by measuring levels of circulating donor molecules and/or by evaluating the size distribution of such molecules. As described further herein, information from circulating donor molecules can be used either alone, or in combination with other markers of transplant injury, such as markers derived from the subject's own tissue (including the subject's immune repertoire) or markers derived from a foreign source such as a pathogen.

Provided herein are methods of determining an origin of a graft injury by discriminating between rejection and infection. In some instances, the fragment length of the donor molecule is used to discriminate between an immunologic rejection (which may be associated with an increase in apoptotic tissue) and an infection (which may manifest in an increase in necrotic tissue). Cell-free DNA is released from both apoptotic and necrotic cells, but the size distribution of the DNA fragments may differ in these two cases.

The methods provided herein may comprise determining a relative level of apoptotic cell death in a donor tissue or organ by evaluating a size profile of circulating DNA (e.g., circulating donor DNA) or other molecule (e.g., nucleic acid, RNA, protein, etc.). The method may further comprise using the level (or relative level) of apoptotic cell death to determine the presence or degree of an immune response to transplanted tissue or a transplanted organ. The immune response may be a cellular immune response and/or an antibody-mediated immune response. Apoptotic cell death usually involves nuclease digestion of the genomic DNA while still bound to nucleosomes prior to release from the cell. Consequently, as a result of apoptosis, the circulating DNA may present as a set of small fragments, often separated by a uniform, or near-uniform periodicity. If the DNA is run on an electrophoretic gel, it may appear as a ladder of fragments of different sizes. The methods provided herein may comprise detecting the level (or relative level) of a set of fragments comprising fragments of size 180 bp, 360 bp, 540 bp, and 720 bp, 900 bp, etc. with the majority of molecules at the smallest sizes. The method may comprise detecting the level (or relative level) of a set of fragments comprising molecules of sizes that are smaller than 200 bp, e.g., a set comprising fragments of sizes of 195 bp, 190 bp, 185 bp, 180 bp, 175 bp, 170 bp, 165 bp, 160 bp, 155 bp, 150 bp, 145 bp, 140 bp, 135 bp, 130 bp, 125 bp, 120 bp, 110 bp, 100 bp, 90 bp, 80 bp, 70 bp, 60 bp, 50 bp, 40 bp, 30 bp, 20 bp, and/or 10 bp, or any combination thereof. The set of fragments may also comprise molecules that are within plus or minus 1, 2, 3, 4, or 5 bp of these values. In some cases, the set of fragments comprises fragments of sizes less than 300 bp, 250 bp, 240 bp, 230 bp, 220 bp, 210 bp, 200 bp, 190 bp, 180 bp, 170 bp, 160 bp, or 150 bp. The set of molecules may be spaced at a periodicity of about 5, 6, 7, 8, 9, 10, 11, 12, 13, 14, 15, 16, 17, 18, 19, 20, 30, 40, 50, 60, 70, 80, 90, 100, 120, 130, 140, 150, 160, 170, 180, 190, 200, 210, or 220 bp. For example, a set of molecules spaced at a periodicity of about 10 bp can comprise fragments of sizes of 10 bp, 20 bp, 30 bp, 40 bp, 50 bp, 60 bp, 70 bp, 80 bp, 90 bp, 100 bp, 110 bp, 120 bp, 130 bp, 140 bp, 150 bp, 160 bp, or 170 bp. In some instances, apoptotic cell death in a donor tissue or organ is characterized by a size profile of donor-derived DNA wherein a majority of DNA fragments are less than about 250 bp. In some instances, the size profile is characterized by an increase in DNA fragments of about 166 bp, when compared to (1) the size profile of DNA from a different tissue type, such as blood; and/or (2) the size profile of DNA from the same tissue type, where the tissue is known to be either healthy or diseased. In other cases, the size profile is characterized by a decrease in DNA fragments with sizes of about 166 bp. In some instances, the size profile is characterized by a decrease in DNA fragments of less than about 120 bp.

The methods provided herein may comprise determining a relative level of apoptotic or necrotic cell death in a donor tissue or organ by evaluating the quantity of circulating RNA derived from the donor tissue or organ. The method may further comprise using the level (or relative level) of apoptotic or necrotic cell death to determine the presence or degree of an immune response to transplanted tissue or a transplanted organ; to determine or predict the degree of tissue or organ rejection or damage; and/or to identify or predict the presence of a pathogenic infection. In some cases, a relative increase in circulating donor RNA indicates a higher risk of rejection. In some cases, a relative decrease in circulating donor RNA indicates a higher risk of rejection. In some cases, a relative increase in circulating donor RNA may indicate a higher risk of pathogenic infection; in other cases, a relative decrease in circulating donor RNA indicates a higher risk of pathogenic infection. The relative increase may be at least 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, 4-fold, 5-fold, 6-fold, 7-fold, 8-fold, 9-fold, 10-fold, 50-fold, 100-fold, 500-fold, 1000-fold or more. Alternatively, the relative decrease is at least 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, 4-fold, 5-fold, 6-fold, 7-fold, 8-fold, 9-fold, 10-fold, 50-fold, 100-fold, 500-fold, 1000-fold or more. In some instances, the relative increase is at most about 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, 4-fold, 5-fold, 6-fold, 7-fold, 8-fold, 9-fold, 10-fold, 50-fold, 100-fold, 500-fold, 1000-fold or more. Alternatively, the relative decrease is at most about 1-fold, 1.25-fold, 1.5-fold, 1.75-fold, 2-fold, 2.25-fold, 2.5-fold, 2.75-fold, 3-fold, 3.25-fold, 3.5-fold, 3.75-fold, 4-fold, 5-fold, 6-fold, 7-fold, 8-fold, 9-fold, 10-fold, 50-fold, 100-fold, 500-fold, 1000-fold or more. The increase or decrease may be relative to the quantity of circulating donor RNA at a particular time point or may occur over a particular time period. For example, the increase or decrease is a 2-fold increase over a 5-day time period. In another example, the increase or decrease is a 2.5-fold increase over, or within, a 1-month time period, a 2-month time period, a 3-month time period, a 4-month time period, a 5-month time period, or a 6-month time period. In some cases, the increase or decrease is a 3-fold increase over, or within, a 1-month time period, a 2-month time period, a 3-month time period, a 4-month time period, a 5-month time period, or a 6-month time period. In some cases, the particular time period is about 0.5, 1, 2, 3, 4, 5, 6, 7, 8, 9, or 10 days; 1, 2, 3, 4, 5, 6, 7, 8, 9, or 10 weeks; 1, 2, 3, 4, 5, 6, 7, 8, 9, or 10 months; or 1 or 2 years, or, in some cases, even longer. In some cases, the particular time period is at most 0.5, 1, 2, 3, 4, 5, 6, 7, 8, 9, or 10 days. Alternatively, the particular time period is at most 1, 2, 3, 4, 5, 6, 7, 8, 9, or 10 months. In some cases, the particular time period is at most 0.5, 1, 2, 3, 4, 5, 6, 7, 8, 9, or 10 years.

The methods provided herein may comprise determining a relative level of necrotic cell death in a donor tissue or organ by evaluating a size profile of circulating DNA (e.g., circulating donor DNA) or of a different molecule (e.g., RNA or other nucleic acid, protein, etc.). The method may further comprise using the level (or relative level) of necrotic cell death to determine the presence or degree of a pathogenic infection associated with transplanted tissue or a transplanted organ. Necrotic cell death is not as orderly as apoptotic cell death. Moreover, DNA released from necrotic cells is generally longer than that released from apoptotic cells. The methods provided herein may comprise detecting the level (or relative level) of a set of fragments comprising fragments of relatively large size. The set of fragments may comprise fragments that are greater than 300 bp, 400 bp, 500 bp, 1000 bp, 1500 bp, 2000 bp, 2500 bp, 3000 bp, 3500 bp, 4000 bp, 4500 bp, 5000 bp, 5500 bp, 6000 bp, 6500 bp, 7000 bp, 7500 bp, 8000 bp, 8500 bp, 9000 bp, 9500 bp, 10000 bp, 10500 bp, 11000 bp, 11500 bp, 12000 bp, 12500 bp, 13000 bp, 13500 bp, 14000 bp, 14500 bp, or 15000 bp.

In some cases, necrotic cell death in a donor tissue or organ is characterized by an increase in smaller-sized DNA fragments, particularly after the donor-derived DNA is digested (e.g., digestion by restriction enzymes). Such increase may be an increase of small-sized donor DNA fragments when compared with digested DNA from healthy tissue, such as healthy recipient tissue. In some cases, such increase is an increase of small-sized DNA fragments when compared with digested donor DNA from a different time-point, or over a particular time period (such as, e.g., 1, 2, 3, 4, 5, 6, 7, 8, 9, 10, 11, or 12 hours; 1, 2, 3, 4, 5, 6, 7, 8, 9, 10, 11, or 12 days; 1, 2, 3, 4, 5, 6, 7, 8, 9, 10, 11, or 12 months; or 1, 2, or 3 years or longer. The increase may be a 1-fold, 2-fold, 3-fold, 4-fold, 5-fold, 6-fold, 7-fold, 8-fold, 9-fold, 10-fold, 50-fold, 100-fold, 500-fold, 1000-fold increase, or even more. In some instances, the smaller-sized DNA fragments are less than about 150 bp, about 140 bp, about 130 bp, about 120 bp, about 110 bp, or about 100 bp. For example, necrotic cell death can be characterized by an increase of DNA fragments of about 120 bp and/or a decrease in DNA fragments of about 166 bp, particularly after digestion.

In some cases, the method comprises identifying whether the fragments have a uniform, or near-uniform, periodicity in size versus whether the sizes of the fragments appear to be more randomly-sized. For example, in some cases, the method may comprise determining whether a size profile of DNA fragments has well-defined peaks of sizes (e.g., as would be more indicative of apoptotic cell death) versus less-defined sizes. The DNA fragments may derive from necrotic DNA if they fail to appear as a ladder with distinct sizes separated by a uniform or near-uniform size periodicity or if they appear as a smear when run on an electrophoretic gel. The methods herein, therefore, may comprise using these factors (size, periodicity, etc.) to determine whether the originating DNA is derived from apoptotic versus necrotic tissue. For example, in some instances, necrotic cell death is characterized by a size profile comprising irregular or randomly-sized DNA fragments, whereas apoptotic cell death is characterized by a size profile comprising DNA fragments of a certain periodicity (e.g., 5 bp, 10 bp, 20 bp, etc). In some cases, the size profile characterizing apoptotic cell death is an even distribution of DNA fragments across a spectrum of sizes. The quantity of donor DNA fragments across a given size profile may vary, on average, by less than 1%, 2%, 3%, 4%, 5%, 6%, 7%, 8%, 9%, 10%, 20%, 30%, 40%, 50%, 60%, 70%, 80%, 90%, 100%, 150%, 200%, 300%, 400%, 500%, or 1000%. For example, for a size profile that contains only DNA fragments that are 50 bp, and 1000 bp in length and where the quantity of 50-bp fragments is half the quantity of 1000 bp fragments, the average quantity variation is less than 100%. In some cases, necrotic cell death is characterized by a size profile comprising indistinguishable or non-discrete DNA fragments (e.g., DNA fragments appear as a smear on an electrophoresis gel).

Methods of obtaining a size profile are described further in other sections herein. Briefly, a size profile can be obtained by any one of a number different techniques, including, but not limited to, sequencing (e.g., paired-end sequencing, single molecule sequencing), electrophoresis (e.g., gel electrophoresis, agarose gel electrophoresis, polyacrylamide electrophoresis, capillary electrophoresis, alkaline gel electrophoresis, pulsed field gel electrophoresis), amplification (e.g., PCR-based amplification, non-PCR based amplification), and arrays. In some instances, devices, including, but not limited to, a sequencing machine, electrophoresis chamber, electrophoresis machine, thermal cycler, PCR machine, plate reader, fluorometer, luminometer, microscope, and computer are used to obtain a size profile.

The method may further comprise obtaining a “Death Mode Ratio” by comparing the relative level of circulating DNA fragments of a certain size (or size pattern, ladder, or profile) associated with apoptosis with the relative level of circulating DNA fragments of a certain size (or size pattern, ladder, or profile) associated with necrosis. Often, the circulating DNA fragments used to obtain the Death Mode Ratio may derive from the donor tissue; but the DNA fragments may also derive from recipient tissue, or some combination of donor and recipient tissue (e.g., necrotic recipient DNA, necrotic donor DNA, apoptotic donor DNA, or apoptotic recipient DNA).

The methods provided herein may comprise correlating a Death Mode Ratio with a control Death Mode Ratio that characterizes a particular known condition, such as a condition or transplant status described herein (e.g., tolerance, immunologic rejection, pathogenic infection (of donor tissue, recipient tissue, or both), specific graft injury, graft injury due to pharmacological agent, etc.). Thus, a method may comprise determining a control Death Mode Ratio, determined by measuring levels of circulating DNA in subjects with a known condition, such as a known immunologic rejection of transplanted tissue or a known pathogenic infection of transplanted tissue. In some cases, the control Death Ratio may be determined in a subject known not to have received a transplant, or who is known to have tolerated a transplant. The subject used to determine the control Death Mode Ratio may be a subject different from the subject used for the Death Mode Ratio or the same as the subject used for the Death Mode Ratio. In some cases, multiple subjects are used to determine the control Death Mode Ratio.

The method may further comprise comparing the Death Mode Ratio of a subject with an unknown condition (e.g., it is unknown whether transplanted tissue has triggered an immunologic rejection) with the control Death Mode Ratio in order to determine, or help determine, whether the subject is experiencing an immunologic rejection or pathogenic infection associated with transplanted tissue. Such method may also determine, or help determine, whether a known case of transplant rejection is worsening or improving. The method may further comprise evaluating or analyzing the comparison of the Death Mode Ratio with the control Death Mode Ratio in order to determine the existence of, risk of, level of, or status of immunologic rejection within the subject with the unknown condition. For example, a Death Mode Ratio of a sample from a transplant recipient can be obtained by determining the quantity of DNA fragments (e.g., donor DNA) that is between 160-170 bp in size and comparing that to the quantity of DNA fragments (e.g., donor DNA) across a broader size range, such as DNA fragments present between 100-250 bp. A Death Mode Ratio of a control sample can be determined in a similar manner. The Death Mode Ratio of the sample from a transplant recipient then can be compared to the Death Mode Ratio of the control sample. A Death Mode Ratio of a sample from the transplant recipient greater than the Death Mode Ratio of the control sample can be indicative of apoptosis within the donor organ or tissue, whereas, a Death Mode Ratio of a sample from the transplant recipient less than the Death Mode Ratio of the control sample can be indicative of necrosis.

Often, the method comprises determining a Death Mode Ratio after determining the relative level of circulating donor nucleic acids within the transplant recipient. For example, the method may comprise (a) evaluating the level of circulating donor nucleic acids (e.g., DNA, RNA) in a transplant recipient and then, if the level has reached a certain threshold level, (b) evaluating a size profile of circulating nucleic acids and/or calculation of a Death Mode Ratio. The threshold level may be a level known to indicate a particular status or outcome: e.g., rejection, threatened rejection, organ failure, organ damage, or risk of the foregoing. In some cases, the threshold level reflects a level of circulating nucleic acids disclosed herein in any section of this disclosure. In some cases, the threshold level is determined on a patient-specific basis. For example, the threshold level may be determined based on a review of a patient's (or other subject's) past history of organ tolerance, rejection or threatened rejection and correlation of such previous events with the level of circulating molecules (e.g, nucleic acids) in the patient. In some instances, an increase in donor-derived molecules (e.g., DNA and/or RNA) and an increase in apoptotic cell death as determined by a Death Mode ratio relative to values determined from previously obtained samples from a transplant recipient are indicative of a rejection or an increased likelihood of rejection in the transplant recipient.

iii. Detecting an Immune Response

In addition to detecting circulating donor molecules, discriminating between rejection and infection may further comprise measuring an immune response in a subject, such as by immune repertoire profiling of T cells and/or B cells. The method may comprise detecting, monitoring, or evaluating an immune response within a transplant recipient, particularly an immune response to transplanted organ, tissue, cells, or molecules. In some cases, an immunological rejection is indicated (or at increased risk) when the immune repertoire profiling reveals the presence of B-cell clones or T cells that are capable of targeting an antigen associated with the transplanted cells, tissues, organ, or molecules. In some cases, an immunological rejection is less indicated (or at reduced risk) when the immune repertoire profiling reveals the absence or reduction of B-cell clones or T cells that are capable of targeting an antigen associated with the transplanted cells, tissues, organ, or molecules.

In some cases, the method comprises determining that a cellular rejection of transplanted tissue or organ is occurring, has an increased risk of occurring, or is worsening, where there is a relative increase in one or both of the following (a) circulating molecules (e.g., DNA) associated with apoptotic donor tissue and (b) immune response as measured by evaluating the immune repertoire. In some cases, the method comprises determining that a cellular rejection of transplanted tissue or organ is not occurring, is at decreased risk of occurring, or is improving, where there is a relative decrease in one or both of the following (a) circulating molecules (e.g., DNA) associated with (or derived from) apoptotic donor tissue and (b) immune response as measured by evaluating the immune repertoire. Although in preferred embodiments the circulating molecules are derived from apoptotic donor tissue, in some cases they may derive from apoptotic recipient tissue.

The method may further comprise detecting, monitoring, or evaluating an immune response to a pathogenic infection associated with the transplant. As described herein, the immune response may be detected, monitored, or evaluated by measuring the immune repertoire. The method may comprise predicting an increased chance that an organ or tissue rejection is due to pathogenic infection, where an increased immune response to a pathogen is detected. For example, the immune repertoire profiling can reveal the presence of (or increased number of, or increased activity of) a large number of B-cell clones producing antibodies to a pathogen, thereby indicating an infection. In some cases, the method may comprise predicting a decreased chance that an organ or tissue rejection is due to infection, where a decreased immune response to a pathogen is detected. For example, immune repertoire profiling can reveal the absence of (or reduction of) T cells (or B cells) targeting a pathogen, thereby indicating the absence of infection by that pathogen and/or possibly the presence of an immunologic rejection episode.

A method that involves detecting an immune response to a pathogen may also comprise using this information along with information regarding relative levels of necrosis or apoptosis to evaluate, predict, monitor or diagnose the risk of, or existence of, a pathogenic infection. For example, the method may comprise determining that there is an increased chance that a transplant rejection is due to infection where a transplant recipient demonstrates a relative increase in one or more of the following: (a) circulating molecules (e.g., DNA) associated with necrosis (e.g., necrotic donor tissue or necrotic recipient tissue) and (b) immune response to a pathogen. Although in preferred embodiments the circulating molecules are derived from necrotic donor tissue; in some cases, they may derive from necrotic recipient tissue.

Detection of the T cell and/or B cell repertoire can comprise sequencing (e.g., high-throughput sequencing), amplifying, and/or quantifying the T cell and/or B cell repertoire. Exemplary methods of measuring the immune repertoire are described in PCT publication No. WO/2011/140433 entitled: Measurement and Comparison of Immune Diversity by High-Throughput Sequencing, filed May 6, 2011.

iv. Detecting Pathogenic Infections

The method may further comprise detecting, monitoring, or evaluating a pathogenic infection within a transplant recipient; particularly a pathogenic infection associated with, or caused by, the transplant (or introduced as a result of the transplantation of a tissue or organ). As described further herein, a pathogenic infection can be detected via numerous methods including by sequencing nucleic acids (e.g., DNA or RNA) or proteins from the pathogen, by amplifying nucleic acids from a pathogen (e.g., by applying a PCR reaction to a sample taken from the transplant recipient, or by using an antibody to detect a particular pathogen). The method may also comprise determining the amount of pathogen (e.g., viral load) in a sample, or otherwise quantifying the pathogen. The method may comprise predicting an increased chance that an organ or tissue rejection is due to pathogenic infection, where a pathogen is detected within a sample taken from a transplant recipient. The method may comprise predicting a decreased chance that an organ or tissue rejection is due to infection, where a pathogen is not detected in a sample taken from a transplant recipient. The method may further comprise using this information along with information regarding relative levels of necrosis or apoptosis, as described herein. For example, the method may comprise determining that there is an increased chance that a transplant rejection is due to infection where a pathogen is detected in a sample taken from a transplant recipient and a transplant recipient demonstrates a relative increase in one or more of the following: (a) circulating molecules (e.g., DNA) associated with necrosis and (b) immune response to a pathogen.

b. Therapeutic Regimens for Organ/Tissue Transplant Recipients

i. General

The methods disclosed herein may further comprise administering, adjusting, or terminating a therapeutic regimen based on the discrimination between rejection and infection. For example, if an infection is indicated, then a therapeutic regimen comprising an anti-microbial (e.g., antibiotic, antiviral, antifungal) is administered, increased, or adjusted (e.g., by making a change in the number or types of pharmacological agents administered). In some cases, if an infection is indicated, then a therapeutic regimen comprising an immunosuppressive drug is reduced, terminated or adjusted (e.g., by making a change in the number or types of pharmacological agents administered). In some cases, if a rejection is indicated, then a therapeutic regimen comprising an immunosuppressive drug is administered, increased or adjusted (e.g., by making a change in the number or types of pharmacological agents administered).

ii. Predicting or Diagnosing Transplant Rejection

This disclosure provides methods of predicting or diagnosing transplant survival (or rejection) in a subject that has received a transplant. The prediction or diagnosis may involve detecting circulating molecules associated with the transplant graft. In many cases, the prediction or diagnosis may take into account other factors as well, such as other indicia of organ failure or reduced function. For example, the prediction or diagnosis may involve monitoring proteinuria in a kidney transplant recipient in addition to the methods described herein.

In some cases, the disclosure provides methods of diagnosing or predicting the length of time that transplanted tissue, organ(s), or cells, will survive, such as the presence of long-term graft survival. By “long-term” graft survival is meant graft survival for at least about five years beyond current sampling, despite the occurrence of one or more prior episodes of acute rejection. In some cases, graft survival is predicted to be at least 1, 2, 3, 4, 5, 6, 7, 8, 9, 10, 11, 12, 13, 14, 15, 20, 25 or 30 years. In some cases, graft survival is predicted to be at least, 1, 2, 3, 4, 5, 6, 7, or 8 weeks, or 1, 2, 3, 4, 5, 6, 7, 8, 10, 11, or 12 months. In other cases, graft survival is predicted to be less than any of these time periods, e.g., less than 1, 2, 3, 4, 5, 6, 7, or 8 weeks, or 1, 2, 3, 4, 5, 6, 7, 8, 10, 11, or 12 months or less than 1, 2, 3, 4, 5, 6, 7, 8, 9, 10, 11, 12, 13, 14, 15, 20, 25 or 30 years.

This disclosure also provides methods of determining or predicting transplant survival following acute rejection. In certain embodiments, transplant survival is determined for patients in which at least one episode of acute rejection has occurred; in some cases, the subject has experienced at least 1, 2, 3, 4, or 5 episodes of rejection of transplanted tissue. Transplant survival is determined or predicted in certain embodiments in the context of transplant therapy, e.g., immunosuppressive therapy, where immunosuppressive therapies are known in the art.

Similarly, the methods provided herein may involve determining, diagnosing, detecting, predicting, or monitoring the risk of, or existence of, a transplant rejection. In yet other embodiments, methods of determining the class and/or severity of rejection (e.g., acute rejection) (and not just the presence thereof) are provided. In some instances, methods of determining the cause of a rejection are provided herein.

In some instances, predicting a status or outcome of an organ transplant comprises predicting a risk of transplant rejection. Alternatively, predicting a status or outcome of an organ transplant comprises predicting or detecting organ failure. Monitoring a status or outcome of an organ transplant may comprise monitoring efficacy of an immunosuppressive regimen. In addition, predicting a status or outcome of an organ transplant may comprise identifying or predicting responders to an immunosuppressive therapy.

In some instances, the method comprises predicting or diagnosing the presence of, risk of, or degree of transplant rejection after detecting an increase in foreign (e.g., donor-derived) molecules, such as circulating donor-derived molecules. A transplant rejection may be indicated, or an increased risk of rejection may be indicted, where donor-derived molecules may increase by at least about 30%, at least about 40%, at least about 50%, at least about 60%, at least about 70%, at least about 80%, or at least about 90%. In other instances, the rejection may be indicated, or at an increased risk, where the donor-derived molecules increase by at least about 1.25-fold, at least about 1.5-fold, at least about 1.75-fold, at least about 2-fold, at least about 2.25-fold, at least about 2.5-fold, at least about 2.75-fold, at least about 3-fold, at least about 3.25-fold, at least about 3.5-fold, at least about 3.75-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 50-fold, or at least about 100-fold. The increase in donor-derived molecules may be detected over a period of time. For example, the increase in donor-derived molecules may occur within about a 2-day period, 5-day period, 10-day period, 14-day period, 21-day period, 28-day period, 1-month period, 2-month period, or 3-month period. In another example, the increase in donor-derived molecules can occur within about a 4-month period, 5-month period, 6-month period, 7-month period, 8-month period, 9-month period, 10-month period, 11-month period, 12-month period, 13-month period, 14-month period, 15-month period, 16-month period, 17-month period, 18-month period, or 24-month period. In some instances, the increase in donor-derived molecules occurs within at most a 1, 2, 3, 4, 5, 6, 7, 8, 9, or 10-month period. In some instances, the increase in donor-derived molecules occurs within at most a 1, 2, 3, 4, 5, 6, 7, 8, 9, or 10-year period. In some instances, an increase in donor-derived molecules relative to a consensus normal or control at about the time of transplantation (e.g., time=0) is indicative of rejection or increased risk of rejection. In some cases, a certain increase in donor-derived molecules is predictive of a transplant rejection with a certainty of at least 25%, 50%, 60%, 70%, 75%, 80%, 85%, 90%, 95%, 96%, 97%, 98%, 99% or 99.5%. The time period may indicate the presence of, or risk of transplant rejection. For example, an increase of about two-fold of donor-derived molecules within a 10 day period may indicate a rejection or increased risk of rejection. As another example, an increase in donor-derived molecules above an established baseline level over several months may indicate a rejection or increased risk of rejection.

In some instances, a gradual increase in foreign molecules (e.g., donor-derived molecules) is indicative of chronic rejection. In some instances, chronic rejection, or risk thereof, is indicated when the foreign molecules increase by less than about 20%, less than about 15%, less than about 12%, less than about 10%, less than about 7%, less than about 5%, over a period of time. Preferably, the foreign molecules increase by less than about 5%, less than about 4%, less than about 3%, less than about 2%, less than about 1%, less than about 0.5%, less than about 0.25% over a period of time. In other instances, the presence of the foreign molecules increases by less than about 1.25-fold, 1.5-fold, less than about 1.75-fold, less than about 2-fold, less than about 2.25-fold, less than about 2.5-fold, less than about 2.75-fold, less than about 3-fold, less than about 3.25-fold, less than about 3.5-fold, less than about 3.75-fold, less than about 4-fold, less than about 5-fold, less than about 6-fold, less than about 7-fold, less than about 8-fold, less than about 9-fold, less than about 10-fold, less than about 15-fold, less than about 20-fold, less than about 50-fold, or less than about 100-fold.

In some instances, the methods disclosed herein predict transplant rejection at least about 1, at least about 2, at least about 3, at least about 4, at least about 5, at least about 6, at least about 7, at least about 8, at least about 9, at least about 10, at least about 11, at least about 12, at least about 13, or at least about 14 days earlier than a biopsy-predicted transplant rejection. Alternatively, the methods disclosed herein predict transplant rejection at least about 1, at least about 2, at least about 3, at least about 4, at least about 5, at least about 6, at least about 7, at least about 8, at least about 9, at least about 10, at least about 11, at least about 12, at least about 13, or at least about 14 weeks earlier than a biopsy-predicted transplant rejection. The methods disclosed herein may predict transplant rejection at least about 1, at least about 2, at least about 3, at least about 4, at least about 5, at least about 6, at least about 7, at least about 8, at least about 9, at least about 10, at least about 11, at least about 12, at least about 13, or at least about 14 months earlier than a biopsy-predicted transplant rejection. The methods disclosed herein may predict transplant rejection at least about 1, 1.25, 1.5, 1.75, 2, 2.25, 2.5, 2.75, 3, 3.25, 3.5, or 3.75 years prior to transplant rejection.

In some instances, the methods disclosed herein predict the cause of a transplant rejection by detecting the presence or absence of foreign nucleic acids, wherein the foreign nucleic acids are not donor-derived nucleic acids. In some instances, the presence of the foreign nucleic acids is indicative of a pathogenic infection. In some instances, the foreign nucleic acids are viral nucleic acids. Alternatively, the foreign nucleic acids are bacterial nucleic acids. In some instances, the presence of the foreign nucleic acids is indicative of an infection within the transplanted tissue or organ. In some instances, the presence of foreign nucleic acids indicates that the rejection is at least partially caused by an infection. In some instances, the infection is a viral infection. In other instances, the infection is a bacterial infection. In some instances, the method further comprises conducting a sequencing reaction on the foreign nucleic acids. In some instances, the absence of foreign nucleic acids is indicates that the rejection is at least partially caused by an immune reaction. In some instances, the immune reaction is a cell-mediated immune response. Alternatively, the immune reaction is an antibody-mediated immune reaction.

In some instances, the methods disclosed herein predict transplant tolerance by monitoring the fold-change of donor nucleic acids, or the percentage change of donor nucleic acids relative to total nucleic acids. In some instances, a fold-increase of not more than about 0.0001-fold, 0.005-fold, 0.01-fold, 0.05-fold, 0.1-fold, 0.15-fold, 0.2-fold, 0.25-fold, 0.3-fold, 0.35-fold, 0.4-fold, 0.45-fold, 0.5-fold, 0.55-fold, 0.6-fold, 0.65-fold, 0.7-fold, 0.75-fold, 0.8-fold, 1-fold, 1.1-fold, 1.2-fold, 1.3-fold, 1.4-fold, 1.5-fold, 1.6-fold, 1.7-fold, 1.8-fold, 1.9-fold, or 2-fold over a period of time, or within a period of time, is indicative of tolerance. In some instances, tolerance is indicated when donor nucleic acids make up not more than about 0.4%, 0.45%, 0.5%, 0.55%, 0.6%, 0.65%, 0.7%, 0.75%, 0.8% of total nucleic acids within a period of time. In some instances, the period of time is over, or within, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. In some instances, the period of time is over, or within, 1, 2, 3, 4, 5, 6, 7, 8, 9, 10, 11, or 12 months. Alternatively, the period of time is over, or within, 1, 2, 3, 4, 5, 6, 7, 8, 9, or 10 years.

In some instances, diagnosing, predicting, or monitoring the status or outcome of a disease or condition comprises determining patient-specific baselines and/or thresholds. For example, the patient-specific baselines and/or thresholds can provide ranges for predicting a transplant rejection. In another example, the patient-specific baselines and/or thresholds can provide ranges for diagnosing a disease or condition. In another example, the baselines and thresholds in a pediatric heart recipient may be higher than in an adult heart recipient. Similarly, the baselines and thresholds in a recipient receiving a partial liver transplant as in living donor transplantation can differ from the baselines and thresholds in a recipient receiving a complete liver. Alternatively, the baselines and thresholds in a recipient receiving a single-lung transplant may differ from the baselines or thresholds in a recipient receiving a double-lung transplant. In some instances, patient-specific baselines and/or thresholds determined at about time of transplantation (e.g., time=0) are compared to baselines and/or thresholds for a group (e.g., children, adults, males, females, ethnic groups). In some instances, comparison of patient-specific baselines and/or thresholds to group-specific baselines and/or thresholds is used to predict outcome and/or guide therapies.

For example, the threshold percentage of donor nucleic acids can be predictive of rejection. In some instances, the threshold percentage of total nucleic acids within a sample that are donor nucleic acids varies depending on the organ. For example, a threshold percentage for a liver transplant where at least about 0.1%, 0.2%, 0.3%, 0.4%, 0.5%, 0.75%, 1%, 1.25%, 1.5%, 2%, 2.2%, 2.5%, 2.7%, 3%, 3.25%, 3.5%, 3.75%, 4%, 4.25%, 4.5%, 4.75%, 5%, 5.25%, 5.5%, 5.75%, or 6% of total nucleic acids are donor nucleic acids is indicative of rejection. In some instances, in a liver transplant, a percentage of total nucleic acids in a sample that are donor nucleic acids that is at least about 1%, 2%, 3%, or 4% may be indicative of rejection. In another example, a percentage for a lung transplant of at least 0.1%, 0.2%, 0.3%, 0.4%, 0.5%, 0.75%, 1%, 1.25%, 1.5%, 2%, 2.25%, 2.5%, 2.75%, 3%, 3.25%, 3.5%, 3.75%, 4%, 4.25%, 4.5%, 4.75% or 5% of total nucleic acids that are donor nucleic acids is indicative of rejection. In some instances, a percentage for a lung transplant of at least about 3% of total nucleic acids that are donor nucleic acids is indicative of rejection. In another example, a rejection is indicated in a kidney or heart transplant where at least 0.1%, 0.2%, 0.3%, 0.4%, 0.5%, 0.75%, 1%, 1.25%, 1.5%, 1.75%, 2%, 2.25%, 2.5%, 2.75%, 3%, 3.25%, 3.5%, 3.75%, or 4% of total nucleic acids within a sample are donor nucleic acids. In some instances, rejection is indicated in a heart or kidney transplant wherein the donor nucleic acids make up at least about 0.2%, 0.3%, 0.5%, 1%, or 2% of the total nucleic acids within a sample. In some instances, rejection is indicated in a heart or kidney transplant wherein the percentage of total nucleic acids within a sample that are donor nucleic acids is within a range of greater than or equal to 0.1% through less than 2%. In some instances, such percentage is within a range of greater than or equal to 0.2% through less than 1.5%. In some instances, such percentage is within a range of greater than or equal to 0.3% through less than 1.5%. In some instances, such percentage is within a range of greater than or equal to 0.4% through less than 1.5%. In some instances, such percentage is within a range of greater than or equal to 0.5% through less than 1.5%.

In some instances, the patient-specific baselines and/or thresholds can provide ranges for predicting transplant tolerance. In some instances, the threshold percentage of donor nucleic acids within a population of total nucleic acids in a sample varies depending on the organ. For example, in a liver transplant, a threshold percentage of total nucleic acids in a sample that are donor nucleic acids that is not more than about 0.5%, 0.75%, 1.0%, 1.1%, 1.2%, 1.3%, 1.4%, 1.5%, 1.6%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%, 2.65%, 2.7%, 2.75%, or 2.8% is indicative of tolerance. In another example, in a lung transplant, a threshold percentage of total nucleic acids that are donor nucleic acids of at least about, or not more than, 0.3%, 0.5%, 0.75%, 1.0%, 1.1%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, or 2% may be indicative of tolerance. In another example, the threshold percentage for a kidney or heart transplant of at least about, or not more than, 0.05%, 0.1%, 0.15%, 0.2%, 0.25%, 0.3%, 0.35%, 0.4%, 0.45%, 0.5%, 0.55%, 0.60%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, or 1.5% donor nucleic acids, within a population of total nucleic acids in a sample, is indicative of tolerance.

The patient-specific baselines and/or thresholds may be determined based on the presence or absence of the foreign molecules. In some instances, the patient-specific baselines and/or thresholds are determined by calculating the absolute percent of foreign molecules in a sample. For example, the presence of at least about 1% of foreign molecules (e.g., donor-derived molecules) in a sample comprising foreign molecules and subject-derived molecules may be predictive of a transplant rejection. In some instances, the patient-specific baseline and/or threshold is when at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 5%, at least about 7%, at least about 10%, at least about 12%, at least about 15%, at least about 20%, at least about 25%, at least about 30%, at least about 35%, at least about 40%, at least about 45%, at least about 50%, at least about 55%, at least about 60%, at least about 65%, or at least about 70% of the total molecules (e.g., nucleic acids, DNA) in a sample are foreign molecules (e.g., nucleic acids, DNA).

iii. Immunosuppressive Therapies

In some instances, the methods, compositions, and systems of the invention disclosed herein are used to determine a therapeutic regimen for a transplant recipient. Determining a therapeutic regimen may comprise administering one or more immunosuppressive therapies. In some cases, the immunosuppressive therapy is administered along with an antimicrobial agent, or instead of an antimicrobial agent. In some cases, the immunosuppressive therapy is administered along with a different pharmaceutical agent (e.g., cancer drug) or in place of such different pharmaceutical agent.

Determining a therapeutic regimen may comprise modifying, recommending, or initiating an immunosuppressive regimen. Modifying a therapeutic regimen may comprise continuing, discontinuing, increasing, or decreasing an immunosuppressive therapy. In some instances, determining a therapeutic regimen comprises preventing, or reducing the risk of, a transplant rejection by administering or modifying an immunosuppressive regimen. In some instances, determining a therapeutic regimen comprises modifying a dosage of a therapeutic drug based on the presence or absence of foreign molecules. Alternatively, determining a therapeutic regimen comprises dose control of a therapeutic drug. Determining a therapeutic regimen may also comprise adjusting the frequency of dosage. In some instances, a therapeutic regimen is administered, modified, or initiated once the donor-derived molecules reach a certain percentage of the total molecules. For example, a therapeutic regime is administered, increased, or initiated when the donor-derived molecule is at least about 0.1%, at least about 0.5%, at least about 1%, at least about 2%, at least about 3%, at least about 4%, at least about 5%, at least about 7%, at least about 10%, at least about 15%, or at least about 20% of the total molecules in the sample. In another example, if the presence of the foreign molecules increases, then the dosage of the therapeutic drug increases. Alternatively, if the presence of the foreign molecules increases, then a new therapeutic drug is administered. The foreign molecules can increase by at least about 30%, at least about 40%, at least about 50%, at least about 60%, at least about 70%, at least about 80%, at least about 90%, at least about 95%, or at least about 100%. The dosage of the therapeutic drug can increase by at least about 5%, at least about 10%, at least about 20%, at least about 30%, at least about 40%, at least about 50%, at least about 60%, at least about 70%, or at least about 80%. In some instances, the dosage of the therapeutic drug can increase by at least about 5-fold, 10-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, or 500-fold. The donor-derived molecules may increase by at least about 30%, at least about 40%, at least about 50%, at least about 60%, at least about 70%, at least about 80%, or at least about 90%. In other instances, the donor-derived molecules may increase by at least about 1.5-fold, at least about 2-fold, at least about 3-fold, at least about 4-fold, at least about 5-fold, at least about 6-fold, at least about 7-fold, at least about 8-fold, at least about 9-fold, or at least about 10-fold.

In some instances, determining a therapeutic regimen comprises terminating or reducing an immunosuppressive regimen. In some instances, a fold-increase in molecules (e.g., donor nucleic acids, donor DNA) of not more than about 0.0001-fold, 0.005-fold, 0.01-fold, 0.05-fold, 0.1-fold, 0.15-fold, 0.2-fold, 0.25-fold, 0.3-fold, 0.35-fold, 0.4-fold, 0.45-fold, 0.5-fold, 0.55-fold, 0.6-fold, 0.65-fold, 0.7-fold, 0.75-fold, 0.8-fold, 1.0-fold, 1.1-fold, 1.2-fold, 1.3-fold, 1.4-fold, 1.5-fold, 1.6-fold, 1.7-fold, 1.8 fold, 1.9-fold, or 2-fold over a period of time is indicative that an immunosuppressive regimen should be reduced or terminated. In some instances, termination or reduction of an immunosuppressive regime is indicated when the quantity of donor nucleic acids reach a certain percentage of the total nucleic acids within a sample within a given period of time, such as greater than, or less than, 0.001%, 0.005%, 0.01%, 0.05%, 0.1%, 0.2%, 0.3%, 0.4%, 0.45%, 0.5%, 0.55%, 0.6%, 0.65%, 0.7%, 0.75%, 0.8%, 0.9%, 1.0%, 1.2%, 1.3%, 1.4%, 1.5%, or 1.8% within a given period of time. In some instances, the given period of time is over, or within, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. In some instances, the given period of time is over, or within, 1, 2, 3, 4, 5, 6, 7, 8, 9, 10, 11, or 12 months. Alternatively, the period of time is over, or within, 1, 2, 3, 4, 5, 6, 7, 8, 9, or 10 years.

The increase in donor-derived molecules may occur over a period of time. For example, the increase in donor-derived molecules may occur in about a 2-day period, 5-day period, 10-day period, 14-day period, 21-day period, 28-day period, 1-month period, 2-month period, or 3-month period.

Alternatively, the increase in donor-derived molecules can occur in about a 4-month period, 5-month period, 6-month period, 7-month period, 8-month period, 9-month period, 10-month period, 11-month period, 12-month period, 13-month period, 14-month period, 15-month period, 16-month period, 17-month period, 18-month period, or 24-month period.

In some cases, if the presence of the foreign molecules increases, then the frequency of the dosage of the therapeutic drug increases. The frequency of the dosage of the therapeutic drug may increase from once a day to twice a day, three times a day, or four times a day. Alternatively, the frequency of the dosage of the therapeutic drug may increase to weekly, biweekly, every other day, daily, or multiple times a day. In some instances, the route of administration of the therapeutic drug is altered in response to an increase in the presence of the foreign molecules. For example, the route of administration of the therapeutic drug can change from oral to intravenous, or from oral to yet a different route of administration such as intraarterial, intramuscular, intracardiac, intraosseous infusion, intrathecal, intraperitoneal, intravesical infusion, intravitreal, nasal, intradermal or subcutaneous.

In some instances, the method comprises decreasing the dosage of a therapeutic drug if the level of the foreign molecules decreases. The foreign molecules can decrease by at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 97%. The dosage of the therapeutic drug can decrease by at least about 5%, at least about 10%, at least about 20%, at least about 30%, at least about 40%, at least about 50%, at least about 60%, at least about 70%, or at least about 80%. In another example, if the presence of the foreign molecules decreases, then the frequency of the dosage of the therapeutic drug decreases. The frequency of the dosage of the therapeutic drug may decrease to four times a day, three times a day, two times a day, one time a day, every other day, biweekly, weekly, or monthly. Alternatively, if the percentage of foreign molecules within a population of total molecules is less than about 10%, less than about 7%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% in the sample, then the therapeutic regimen is decreased or terminated. Alternatively, if the percentage of foreign molecules is less than about 10%, less than about 7%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% in the sample, then the frequency of dosage of the therapeutic drug is decreased. In some instances, the route of administration of the therapeutic drug is altered in response to a decrease in the presence of the foreign molecules. For example, the route of administration of the therapeutic drug can change from oral to intravenous, or from oral to yet a different route of administration such as intraarterial, intramuscular, intracardiac, intraosseous infusion, intrathecal, intraperitoneal, intravesical infusion, intravitreal, nasal, intradermal or subcutaneous.

An immunosuppressive regimen may comprise one or more immunosuppressive therapies. Examples of immunosuppressive therapies include, but are not limited to, glucocorticoids, cytostatics, antibodies, drugs acting on immunophilins, other drugs, and any combination thereof. In some instances, the immunosuppressive therapy may comprise a glucocorticoid. Glucocorticoids (GC) are a class of steroid hormones that bind to the glucocorticoid receptor (GR). Examples of glucocorticoids include, but are not limited to, hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (DOCA), and aldosterone.

Alternatively, the immunosuppressive therapy may comprise a cytostatic drug. Cytostatic drugs may inhibit cell division and may affect the proliferation of both T cells and B cells. Cytostatic drugs may be alkylating agents, antimetabolites, or cytotoxic antibiotics. The alkylating agents used in immunotherapy may be nitrogen mustards (cyclophosphamide), nitrosoureas, platinum compounds, and others.

Cytostatic drugs such as antimetabolites may interfere with the synthesis of nucleic acids. In some instances, the antimetabolite is an inhibitor of de novo purine synthesis, such as mycophenolic acid (MPA) or mycophenolate mofetil (MMF, CellCept, Myfortic) or an inhibitor of de novo pyramidine synthesis, such as leflunomide. Antimetabolites may include folic acid analogues, such as methotrexate; purine analogues such as azathioprine and mercaptopurine; pyrimidine analogues; and protein synthesis inhibitors. Methotrexate is a folic acid analogue. Methotrexate may bind to dihydrofolate reductase and prevents synthesis of tetrahydrofolate. Azathioprine may be used to control transplant rejection reactions. It may be nonenzymatically cleaved to mercaptopurine that acts as a purine analogue and an inhibitor of DNA synthesis. Mercaptopurine itself can also be administered directly.

Cytotoxic antibiotics are another example of an immunosuppressive therapy. Cytotoxic antibiotics may prevent the clonal expansion of lymphocytes in the induction phase of the immune response, thereby affecting both the cell and the humoral immunity. Examples of cytotoxic antibiotics include, but are not limited to, dactinomycin, anthracyclines, mitomycin C, bleomycin, and mithramycin.

Antibodies are sometimes used as an immunosuppressive therapy. Heterologous polyclonal antibodies may be obtained from the serum of animals (e.g., rabbit, horse), and injected with the patient's thymocytes or lymphocytes. Polyclonal antibodies may inhibit T lymphocytes and may cause their lysis, which may be complement-mediated cytolysis and cell-mediated opsonization followed by removal of reticuloendothelial cells from the circulation in the spleen and liver. Examples of polyclonal antibodies include, but are not limited to, atgam and thymoglobuline. Polyclonal antibodies may be administered with highly-purified serum fractions. Alternatively, polyclonal antibodies may be administered in combination with other immunosuppressants, for example, calcineurin inhibitors, cytostatics and corticosteroids. Preferably, combination therapy comprises antibodies and ciclosporin.

Monoclonal antibodies are another example of antibody therapies and may be directed towards exactly defined antigens. Examples of monoclonal antibodies include, but are not limited to, IL-2 receptor-(CD25-) and CD3-directed antibodies. Muromonab-CD3 is a murine anti-CD3 monoclonal antibody of the IgG2a type that may prevent T-cell activation and proliferation by binding the T-cell receptor complex present on all differentiated T cells. Monoclonal antibody may be administered to control the steroid- and/or polyclonal antibodies-resistant acute rejection episodes. Monoclonal antibodies may also be used prophylactically in transplantations.

In some instances, the immunosuppressive therapy may comprise an anti-IL-2 antibody. Examples of anti-IL-2 antibodies include basiliximab (Simulect) and daclizumab (Zenapax). They may be used in the prophylaxis of the acute organ rejection.

Additional examples of immunosuppressive therapy or therapies comprise drugs that act on immunophilins. Drugs that act on immunophilins include ciclosporin, tacrolimus, and sirolimus. Tacrolimus (trade name Prograf) is macrolide lactone and is a product of the bacterium Streptomyces tsukubaensis. Preferably, tacrolimus is used in liver and kidney transplantations. Alternatively, tacrolimus may be used heart, lung, and heart/lung transplantations. Like tacrolimus, ciclosporin is an immunosuppressive therapy. It is a cyclic fungal peptide, composed of 11 amino acids. Ciclosporin (or cyclosporin) may bind to the cytosolic protein cyclophilin (an immunophilin) of immunocompetent lymphocytes, especially T-lymphocytes. This complex of ciclosporin and cyclophilin inhibits the phosphatase calcineurin, which under normal circumstances induces the transcription of interleukin-2 Cyclosporin may also inhibit lymphokine production and interleukin release, leading to a reduced function of effector T-cells. Ciclosporin can be used in the treatment of acute rejection reactions. Sirolimus (rapamycin, trade name Rapamune) is a macrolide lactone and is produced by the actinomycete bacterium Streptomyces hygroscopicus. Sirolimus may be used to prevent rejection reactions. Although sirolimus is a structural analogue of tacrolimus, it may act somewhat differently. Sirolimus may affect the second phase, namely signal transduction and lymphocyte clonal proliferation and may inhibit mTOR. Therefore, sirolimus may act synergistically with ciclosporin and tacrolimus. Also, sirolimus may indirectly inhibit several T lymphocyte-specific kinases and phosphatases, hence preventing their transition from G₁ to S phase of the cell cycle. In a similar manner, sirolimus may prevent B cell differentiation into plasma cells, reducing production of IgM, IgG, and IgA antibodies.

Additional immunosuppressive drugs or therapies may comprise interferons, opioids, TNF-α (tumor necrosis factor-alpha) binding protein, mycophenolic acid, and small biological agents. Interferons, such as IFN-β and IFN-γ may be used as immunosuppressive therapy. IFN-β may suppress the production of TH1 cytokines and the activation of monocytes. IFN-γ may trigger lymphocytic apoptosis.

Alternatively, immunosuppressive drugs or therapies may comprise inhibition or blockage of T-cell stimulation. In some instances, the immunosuppressive drugs or therapies bind CD80 or CD86. Examples of immunosuppressive drugs or therapies that bind to CD80 and/or CD86 include, but are not limited to, anti-CD80 antibodies, anti-CD86 antibodies, CTLA4-Ig, XENP9523, and belatacept. In other instances, the immunosuppressive drug or therapy comprises a fusion protein. The fusion protein may comprise an Fc fragment of a human immunoglobulin linked to an extracellular domain of CTLA-4. Non-limiting examples of fusion proteins include alefacept (Amevive®), etanercept (Enbrel®), and atacicept. In some instances, the immunosuppressive drug or therapy is belatacept (Nulojix®).

TNF-α (tumor necrosis factor-alpha) binding proteins may also act as immunosuppressants. A TNF-α (tumor necrosis factor-alpha) binding protein may be a monoclonal antibody or a circulating receptor such as infliximab (Remicade), etanercept (Enbrel), or adalimumab (Humira) that may bind to TNF-α and may prevent TNF-α from inducing the synthesis of IL-1 and IL-6 and the adhesion of lymphocyte-activating molecules. TNF or the effects of TNF may also be suppressed by various natural compounds, including curcumin (an ingredient in turmeric) and catechins (in green tea).

Another type of immunosuppressive therapy is mycophenolic acid. Mycophenolic acid may act as a non-competitive, selective, and reversible inhibitor of Inosine-5′-monophosphate dehydrogenase (IMPDH), which is a key enzyme in the de novo guanosine nucleotide synthesis. Lymphocytes B and T are very dependent on de novo guanosine nucleotide synthesis.

Small biological agents such as fingolimod and myriocin may also be used as immunosuppressive therapies. Fingolimod is a synthetic immunosuppressant. It may increase the expression or changes the function of certain adhesion molecules (α4/β7 integrin) in lymphocytes, so they accumulate in the lymphatic tissue (lymphatic nodes) and their number in the circulation is diminished. Myriocin, also known as antibiotic ISP-1 and thermozymocidin, is an atypical amino acid and an antibiotic derived from certain thermophilic fungi. Among the producing strains are Mycelia sterilia and Isaria sinclairii. Myriocin may inhibit serine palmitoyltransferase, the first step in sphingosine biosynthesis. Myriocin may inhibit the proliferation of an IL-2-dependent mouse cytotoxic T cell line and possesses immunosuppressant activity.

iv. Reducing the Risk of or Avoiding, Over-Suppression

In some instances, a subject is treated with a therapeutic drug and the dosage or frequency of dosage of the therapeutic drug is higher or more frequent than what is necessary to achieve a therapeutic or prophylactic effect. In a transplant recipient, the over-dosage or high frequency of dosage results in over-suppression of the immune system. Detection of the foreign molecules (e.g., donor-derived molecules) can provide insight into or determine the effective therapeutic dosage or frequency of dosage of an immunosuppressive drug.

In some instances, diagnosing, predicting, or monitoring the status or outcome of a disease comprises preventing over-suppression therapy based on the presence or absence of foreign molecules (e.g., donor-derived molecules). Preventing over-suppression therapy may comprise maintaining, adjusting, or terminating an immunosuppressive therapy. For example, if the presence of the foreign molecules decreases, then the dosage or the frequency of dosage of an immunosuppressive therapy is reduced, thereby preventing over-suppression therapy. Similarly, if the presence of the foreign molecules is unchanged over a period of time, then the dosage or the frequency of dosage of the immunosuppressive therapy can be reduced. Following the reduction in the dosage or dosage frequency, the presence of the foreign molecules may be assayed. The detection of the foreign molecules can be used to determine the minimal effective dosage and frequency of dosage that is necessary to achieve therapeutic efficacy and prevent over-suppression therapy. Often, the level of foreign molecules in a subject may be monitored over time in order to determine a patient-specific threshold or baseline.

v. Reducing the Risk of or Avoiding, Toxicity

In some instances, a subject is treated with a therapeutic drug and the dosage or frequency of dosage of the therapeutic drug is toxic to the subject. In a transplant recipient, the over-dosage or high frequency of dosage results in increased toxicity or risk of death. Detection of the foreign molecules can provide insight into or determine the effective therapeutic dosage or frequency of dosage of an immunosuppressive drug to minimize or reduce toxicity.

In some instances, diagnosing, predicting, or monitoring the status or outcome of a disease comprises preventing or reducing toxicity of a therapeutic drug based on the presence or absence of foreign molecules. Preventing or reducing toxicity may comprise maintaining, adjusting, or terminating an immunosuppressive therapy. For example, if the presence of the foreign molecules decreases, then the dosage or the frequency of dosage of an immunosuppressive therapy is reduced, thereby preventing or reducing toxicity. Similarly, if the presence of the foreign molecules is unchanged over a period of time, then the dosage or the frequency of dosage of the immunosuppressive therapy can be reduced. Following the reduction in the dosage or dosage frequency, the presence of the foreign molecules may be assayed. The detection of the foreign molecules can be used to determine the minimal effective dosage and frequency of dosage that is necessary to achieve therapeutic efficacy and prevent or reducing toxicity. Often, the level of foreign molecules in a subject may be monitored over time in order to determine a patient-specific threshold or baseline.

c. Tissue and Organ Types

As described further herein, heterogeneous samples taken from a transplant recipient may comprise a mix of molecules, some derived from the recipient and others derived from the transplanted organ(s), tissue(s) or cell(s). The transplant organ(s), tissue(s) or cell(s) may be allogeneic or xenogeneic, such that the grafts may be allografts or xenografts. In some instances, the transplant organ(s), tissue(s) or cell(s) are allogeneic. In other instances, the transplant organ(s), tissue(s) or cell(s) are xenogeneic.

In some cases, the transplant organ(s), tissue(s) or cell(s) are derived from the subject; but in most cases, the transplant organ(s), tissue(s) or cell(s) are derived from a different subject. In some cases, the transplant organ(s), tissue(s) or cell(s) are derived from a human, mammal, non-human mammal, ape, orangutan, monkey, chimpanzee, cow, pig, horse, rodent, bird, reptile, or other animal.

The transplant graft may be any solid organ, hollow organ, bone marrow or skin transplant. The transplanted tissue can be whole organs, or portions of organs. Examples of organ transplants that can be analyzed by the methods described herein include but are not limited to kidney transplant, pancreas transplant, liver transplant, heart transplant, lung transplant, intestine transplant, bladder transplant, pancreas after kidney transplant, simultaneous pancreas-kidney transplant, blood transfusion, or bone marrow transplantation. The organ transplant may also be part of reconstructive surgery, such as a cartilage or tendon transplant.

Examples of donor organs (or portions of organs) include, but are not limited to: adrenal gland, appendix, bladder, brain, ear, esophagus, eye, gall bladder, heart, kidney, large intestine, liver, lung, mouth, muscle, nose, pancreas, parathyroid gland, pineal gland, pituitary gland, skin, small intestine, spleen, stomach, thymus, thyroid gland, trachea, uterus, vermiform appendix, cornea, skin, heart valve, artery, or vein. In some cases, the organ is a gland organ. For example, the organ may be an organ of the digestive or endocrine system; in some cases, the organ can be both an endocrine gland and a digestive organ. In some cases, the organ may be derived from endoderm, ectoderm, primitive endoderm, or mesoderm. In other instances, donor cells are derived from the bone marrow, particularly where the heterogeneous samples are from a bone marrow transplant recipient.

In some cases, organ, tissue or cell transplant (or foreign molecules derived therefrom) is an intact organ, a fragment (or portion) of an intact organ, a disrupted organ, or a cell from any of the organs disclosed herein. Donor cells may be derived from any of the donor organs disclosed herein (e.g., pancreatic cell, hepatic cell, glioma, etc). For example, the transplanted tissue may comprise disrupted brain tissue and may comprise neurons (e.g., nerve cells) and/or glial cells (e.g., astrocytes, oligodendrocytes, ependymal cells). The transplanted tissue may also comprise stem cells (e.g., multipotent stem cells, pluripotent stem cells, neuronal stem cells, heart stem cells, induced pluripotent stem cells, embryonic stem cells, cells derived from cord blood, etc.). In some cases, the transplanted organ, tissues or cells may comprise cholecystocytes, cardiomyocytes, valves, glomerulus cells (e.g., parietal, podocyte), kidney proximal tubule brush border cells, Loop of Henle thin segment cell, thick ascending limb cell, kidney distal tubule cell, kidney collecting ductal cell, or interstitial kidney cell, enterocytes, goblet cells, enterocytes, caveolated tuft cells, enteroendocrine cells, ganglion neuron, parenchymal cells, non-parenchymal cells, hepatocytes, sinusoidal endothelial cells, kupffer cells, hepatic stellate cells, tendon, cartilage, bone, blood, lymph, myocytes, muscle fibres, pancreatic beta cells, endothelial cells, or exocrine cells.

Exemplary tissues include but are not limited to: connective tissue, epithelial tissue, muscular tissue, nervous tissue, fat tissue, dense fibrous tissue, skeletal muscle, cardiac muscle, or smooth muscle. The muscle tissue may comprise muscle fibres or myocytes. In some cases, the tissue is a bone or tendon (both referred to as musculoskeletal grafts).

Often, the donor tissue is derived from an adult. The donor tissue, organ, or cells may also be derived from a fetus, embryo, embryonic stem cells, induced pluripotent stem cells, child, or teenager. The donor tissue may be from a male or a female.

The donor organ, tissue, or cells may be derived from a subject who has certain similarities or compatibilities with the recipient subject. For example, the donor organ, tissue, or cells may be derived from a donor subject who is age-matched, ethnicity-matched, gender-matched, blood-type compatible, or HLA-type compatible with the recipient subject.

d. Transplant Recipients

The subjects disclosed anywhere in this disclosure including the transplant recipients described herein may be mammals or non-mammals. Preferably the subjects are a mammal, such as, a human, non-human primate (e.g., apes, monkeys, chimpanzees), cat, dog, rabbit, goat, horse, cow, pig, and sheep. Even more preferably, the subject is a human. The subject may be male or female; the subject may be a fetus, infant, child, adolescent, teenager or adult. Non-mammals include, but are not limited to, reptiles, amphibians, avians, and fish. A reptile may be a lizard, snake, alligator, turtle, crocodile, and tortoise. An amphibian may be a toad, frog, newt, and salamander. Examples of avians include, but are not limited to, ducks, geese, penguins, ostriches, and owls. Examples of fish include, but are not limited to, catfish, eels, sharks, and swordfish.

Often, the subject is a patient or other individual undergoing a treatment regimen, or being evaluated for a treatment regimen (e.g., immunosuppressive therapy). However, in some cases, the subject is not undergoing a treatment regimen. A feature of the graft tolerant phenotype detected or identified by the subject methods is that it is a phenotype which occurs without immunosuppressive therapy, e.g., it is present in a host that is not undergoing immunosuppressive therapy such that immunosuppressive agents are not being administered to the host.

### III. Pathogenic Infections

As described herein, this disclosure provides methods of detecting, monitoring, quantitating, or evaluating a presence of pathogen-derived molecules (e.g., viral, bacterial, fungal) in order to discriminate between a rejection and an infection in a recipient of a tissue or organ transplant. This disclosure also provides general methods of detecting monitoring, quantitating, or evaluating pathogen-derived molecules outside of an organ or tissue transplant setting.

The methods may comprise detecting the pathogen in a subject who shows signs or symptoms of an infection, who has been exposed to an infectious pathogen, who is suspected of having a pathogenic infection, who is at risk of having a pathogenic infection, who has undergone surgery or who is suffering from a disease or disorder (e.g., cancer). In other cases, the method may comprise detecting a pathogen in a healthy subject, or a subject who shows no signs or symptoms of disease. The pathogen-derived molecules can be detected by any methods known in the art and can include amplifying, sequencing, detecting by antibody, and/or quantifying the pathogen-derived molecules.

Often, the presence of pathogen-derived molecules is indicative of an infection. This disclosure also provides non-invasive diagnostics for the detection of infection by organisms using the methods described herein to detect the “foreign genome” within the host genome. For example, some viruses, such as retroviruses or lentiviruses, are able to integrate into a host genome; the integrated viral nucleic acids may then become part of the circulating molecules within the bodily fluid of a subject. By regular monitoring of circulating nucleic acids in bodily fluid (e.g., blood, or urine) using the genotyping methods described herein, one can detect the presence of infection by a virus or microorganism (e.g., bacteria, fungi, archae, protists).

In some instances, the methods (e.g., a method of discriminating between a rejection and infection) may comprise detection of a foreign (e.g., pathogen-derived, subject-derived, donor-derived, fetal-derived, cancer-derived) molecule. Detection of the foreign molecules may comprise the attachment of one or more barcodes to the foreign molecules. The barcode can comprise a unique sequence and/or primer sequence. The barcode can be used to amplify, sequence, quantify, and/or distinguish the foreign molecules.

Detection of the foreign molecules may comprise the use of foreign-molecule specific primers. For example, the foreign-molecule specific primers comprise a pathogen-specific primer (e.g., viral or bacterial-specific primer), donor-specific primer, fetal-specific primer, or cancer-specific primer.

In some instances, the heterogeneous sample is from a subject suffering from a disease or condition caused by a pathogen and the heterogeneous sample comprises foreign molecules derived from a pathogen and molecules derived from the subject. In some instances, the pathogen is a bacterium, fungi, virus, or protozoan.

Exemplary pathogens include but are not limited to: Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Vibrio, or Yersinia. In some cases, the disease or condition caused by the pathogen is tuberculosis and the heterogeneous sample comprises foreign molecules derived from the bacterium Mycobacterium tuberculosis and molecules derived from the subject. In some instances, the disease or condition is caused by a bacterium is tuberculosis, pneumonia, which can be caused by bacteria such as Streptococcus and Pseudomonas, a foodborne illness, which can be caused by bacteria such as Shigella, Campylobacter and Salmonella, and an infection such as tetanus, typhoid fever, diphtheria, syphilis and leprosy. The disease or condition may be bacterial vaginosis, a disease of the vagina caused by an imbalance of naturally occurring bacterial flora. Alternatively, the disease or condition is a bacterial meningitis, a bacterial inflammation of the meninges (e.g., the protective membranes covering the brain and spinal cord). Other diseases or conditions caused by bacteria include, but are not limited to, bacterial pneumonia, a urinary tract infection, bacterial gastroenteritis, and bacterial skin infection. Examples of bacterial skin infections include, but are not limited to, impetigo which may be caused by Staphylococcus aureus or Streptococcus pyogenes; erysipelas which may be caused by a streptococcus bacterial infection of the deep epidermis with lymphatic spread; and cellulitis which may be caused by normal skin flora or by exogenous bacteria.

The pathogen may be a fungus, such as, Candida, Aspergillus, Cryptococcus, Histoplasma, Pneumocystis, and Stachybotrys. Examples of diseases or conditions caused by a fungus include, but are not limited to, jock itch, yeast infection, ringworm, and athlete's foot.

The pathogen may be a virus. Examples of viruses include, but are not limited to, adenovirus, coxsackievirus, Epstein-Barr virus, Hepatitis virus (e.g., Hepatitis A, B, and C), herpes simplex virus (type 1 and 2), cytomegalovirus, herpes virus, HIV, influenza virus, measles virus, mumps virus, papillomavirus, parainfluenza virus, poliovirus, respiratory syncytial virus, rubella virus, and varicella-zoster virus. Examples of diseases or conditions caused by viruses include, but are not limited to, cold, flu, hepatitis, AIDS, chicken pox, rubella, mumps, measles, warts, and poliomyelitis.

The pathogen may be a protozoan, such as Acanthamoeba (e.g., A. astronyxis, A. castellanii, A. culbertsoni, A. hatchetti, A. polyphaga, A. rhysodes, A. healyi, A. divionensis), Brachiola (e.g., B connori, B. vesicularum), Cryptosporidium (e.g., C. parvum), Cyclospora (e.g., C. cayetanensis), Encephalitozoon (e.g., E. cuniculi, E. hellem, E. intestinalis), Entamoeba (e.g., E. histolytica), Enterocytozoon (e.g., E. bieneusi), Giardia (e.g., G. lamblia), Isospora (e.g, I. belli), Microsporidium (e.g., M. africanum, M. ceylonensis), Naegleria (e.g., N. fowleri), Nosema (e.g., N. algerae, N. ocularum), Pleistophora, Trachipleistophora (e.g., T. anthropophthera, T. hominis), and Vittaforma (e.g., V. corneae).

The detection of foreign molecules (e.g., donor-derived molecules) in a subject suffering from a pathogenic infection may be used in the diagnosis, prediction, or monitoring of a status or outcome of a pathogenic infection. For example, diagnosing, predicting, or monitoring a status or outcome of a pathogenic infection may comprise diagnosing or detecting a pathogenic infection. In other instances, diagnosing, predicting, or monitoring a status or outcome of a pathogenic infection may comprise predicting the risk of recurrence. Alternatively, diagnosing, predicting, or monitoring a status or outcome of a pathogenic infection may comprise predicting mortality or morbidity. Diagnosing, predicting, or monitoring a status or outcome of a pathogenic infection may comprise treating a pathogenic infection or preventing disease progression. In addition, diagnosing, predicting, or monitoring a status or outcome of a pathogenic infection may comprise identifying or predicting responders to an antimicrobial therapy.

In some instances, diagnosing, predicting, or monitoring may comprise determining a therapeutic regimen. Determining a therapeutic regimen may comprise administering an anti-microbial therapy. Alternatively, determining a therapeutic regimen may comprise modifying, recommending, continuing or discontinuing an antimicrobial regimen. An antimicrobial regimen may comprise one or more antimicrobial therapies.

An antimicrobial is a substance that kills or inhibits the growth of microorganisms such as bacteria, fungi, virus, or protozoans. Antimicrobial drugs either kill microbes (microbicidal) or prevent the growth of microbes (microbiostatic). There are mainly two classes of antimicrobial drugs, those obtained from natural sources (e.g., antibiotics, protein synthesis inhibitors (such as aminoglycosides, macrolides, tetracyclines, chloramphenicol, polypeptides)) and synthetic agents (e.g., sulphonamides, cotrimoxazole, quinolones). In some instances, the antimicrobial drug is an antibiotic, anti-viral, anti-fungal, anti-malarial, anti-tuberculosis drug, anti-leprotic, or anti-protozoal.

Antibiotics are generally used to treat bacterial infections. Antibiotics may be divided into two categories: bactericidal antibiotics and bacteriostatic antibiotics. Generally, bactericidals may kill bacteria directly where bacteriostatics may prevent them from dividing. Antibiotics may be derived from living organisms or may include synthetic antimicrobials, such as the sulfonamides. Antibiotics may include aminoglycosides, such as amikacin, gentamicin, kanamycin, neomycin, netilmicin, tobramycin, and paromomycin. Alternatively, antibiotics may be ansamycins (e.g., geldanamycin, herbimycin), cabacephems (e.g., loracarbef), carbapenems (e.g., ertapenem, doripenem, imipenem, cilastatin, meropenem), glycopeptides (e.g., teicoplanin, vancomycin, telavancin), lincosamides (e.g., clindamycin, lincomycin, daptomycin), macrolides (e.g., azithromycin, clarithromycin, dirithromycin, erythromycin, roxithromycin, troleandomycin, telithromycin, spectinomycin, spiramycin), nitrofurans (e.g., furazolidone, nitrofurantoin), and polypeptides (e.g., bacitracin, colistin, polymyxin B).

In some instances, the antibiotic therapy includes cephalosporins such as cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, and ceftobiprole.

The antibiotic therapy may also include penicillins. Examples of penicillins include amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, temocillin, and ticarcillin.

Alternatively, quinolines may be used to treat a bacterial infection. Examples of quinilones include ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, and temafloxacin.

In some instances, the antibiotic therapy comprises a combination of two or more therapies. For example, amoxicillin and clavulanate, ampicillin and sulbactam, piperacillin and tazobactam, or ticarcillin and clavulanate may be used to treat a bacterial infection.

Sulfonamides may also be used to treat bacterial infections. Examples of sulfonamides include, but are not limited to, mafenide, sulfonamidochrysoidine, sulfacetamide, sulfadiazine, silver sulfadiazine, sulfamethizole, sulfamethoxazole, sulfanilimide, sulfasalazine, sulfisoxazole, trimethoprim, and trimethoprim-sulfamethoxazole (co-trimoxazole) (tmp-smx).

Tetracyclines are another example of antibiotics. Tetracyclines may inhibit the binding of aminoacyl-tRNA to the mRNA-ribosome complex by binding to the 30S ribosomal subunit in the mRNA translation complex. Tetracyclines include demeclocycline, doxycycline, minocycline, oxytetracycline, and tetracycline. Additional antibiotics that may be used to treat bacterial infections include arsphenamine, chloramphenicol, fosfomycin, fusidic acid, linezolid, metronidazole, mupirocin, platensimycin, quinupristin/dalfopristin, rifaximin, thiamphenicol, tigecycline, tinidazole, clofazimine, dapsone, capreomycin, cycloserine, ethambutol, ethionamide, isoniazid, pyrazinamide, rifampicin, rifamycin, rifabutin, rifapentine, and streptomycin.

Antiviral therapies are a class of medication used specifically for treating viral infections. Like antibiotics, specific antivirals are used for specific viruses. They are relatively harmless to the host, and therefore can be used to treat infections. Antiviral therapies may inhibit various stages of the viral life cycle. For example, an antiviral therapy may inhibit attachment of the virus to a cellular receptor. Such antiviral therapies may include agents that mimic the virus associated protein (VAP and bind to the cellular receptors. Other antiviral therapies may inhibit viral entry, viral uncoating (e.g., amantadine, rimantadine, pleconaril), viral synthesis, viral integration, viral transcription, or viral translation (e.g., fomivirsen). In some instances, the antiviral therapy is a morpholino antisense. Antiviral therapies should be distinguished from viricides, which actively deactivate virus particles outside the body.

Many of the antiviral drugs available are designed to treat infections by retroviruses, mostly HIV. Antiretroviral drugs may include the class of protease inhibitors, reverse transcriptase inhibitors, and integrase inhibitors. Drugs to treat HIV may include a protease inhibitor (e.g., invirase, saquinavir, kaletra, lopinavir, lexiva, fosamprenavir, norvir, ritonavir, prezista, duranavir, reyataz, viracept), integrase inhibitor (e.g., raltegravir), transcriptase inhibitor (e.g., abacavir, ziagen, agenerase, amprenavir, aptivus, tipranavir, crixivan, indinavir, fortovase, saquinavir, Intelence™, etravirine, isentress, viread), reverse transcriptase inhibitor (e.g., delavirdine, efavirenz, epivir, hivid, nevirapine, retrovir, AZT, stuvadine, truvada, videx), fusion inhibitor (e.g., fuzeon, enfuvirtide), chemokine coreceptor antagonist (e.g., selzentry, emtriva, emtricitabine, epzicom, or trizivir). Alternatively, antiretroviral therarapies may be combination therapies, such as atripla (e.g., efavirenz, emtricitabine, and tenofovira disoproxil fumarate) and completer (embricitabine, rilpivirine, and tenofovir disoproxil fumarate). Herpes viruses, best known for causing cold sores and genital herpes, are usually treated with the nucleoside analogue acyclovir. Viral hepatitis (A-E) are caused by five unrelated hepatotropic viruses and are also commonly treated with antiviral drugs depending on the type of infection. Influenza A and B viruses are important targets for the development of new influenza treatments to overcome the resistance to existing neuraminidase inhibitors such as oseltamivir.

In some instances, the antiviral therapy may comprise a reverse transcriptase inhibitor. Reverse transcriptase inhibitors may be nucleoside reverse transcriptase inhibitors or non-nucleoside reverse transcriptase inhibitors. Nucleoside reverse transcriptase inhibitors may include, but are not limited to, combivir, emtriva, epivir, epzicom, hivid, retrovir, trizivir, truvada, videx ec, videx, viread, zerit, and ziagen. Non-nucleoside reverse transcriptase inhibitors may comprise edurant, intelence, rescriptor, sustiva, and viramune (immediate release or extended release).

Protease inhibitors are another example of antiviral drugs and may include, but are not limited to, agenerase, aptivus, crixivan, fortovase, invirase, kaletra, lexiva, norvir, prezista, reyataz, and viracept. Alternatively, the antiviral therapy may comprise a fusion inhibitor (e.g., enfuviride) or an entry inhibitor (e.g., maraviroc).

Additional examples of antiviral drugs include abacavir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, fusion inhibitors, ganciclovir, ibacitabine, imunovir, idoxuridine, imiquimod, indinavir, inosine, integrase inhibitor, interferons (e.g., interferon type I, II, III), lamivudine, lopinavir, loviride, maraviroc, moroxydine, methisazone, nelfinavir, nevirapine, nexavir, nucleoside analogues, oseltamivir, peg-interferon alfa-2a, penciclovir, peramivir, pleconaril, podophyllotoxin, protease inhibitors, raltegravir, reverse transcriptase inhibitors, ribavirin, rimantadine, ritonavir, pyramidine, saquinavir, stavudine, tea tree oil, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

An antifungal drug is medication that may be used to treat fungal infections such as athlete's foot, ringworm, candidiasis (thrush), serious systemic infections such as cryptococcal meningitis, and others. Antifungals work by exploiting differences between mammalian and fungal cells to kill off the fungal organism. Unlike bacteria, both fungi and humans are eukaryotes. Thus, fungal and human cells are similar at the molecular level, making it more difficult to find a target for an antifungal drug to attack that does not also exist in the infected organism.

Antiparasitics are a class of medications which are indicated for the treatment of infection by parasites, such as nematodes, cestodes, trematodes, infectious protozoa, and amoebae. Like antifungals, they must kill the infecting pest without serious damage to the host.

### IV. Additional Applications

a. Cancer

This disclosure provides highly sensitive, non-invasive diagnostics for the detection, monitoring or prognosis of cancer using partial or whole genome analysis of circulating nucleic acids derived from tumors as compared to the patient's genome. In some instances, the presence of sequences differing from a patient's normal genotype can be used to detect disease. In cancer, genetic variations such as gene mutations or copy number changes can be predictive of the advance of the disease.

This disclosure provides methods for the detection, monitoring and/or prognosis of cancer in a subject. In some instances, the method comprises detection and/or quantifying a foreign molecule. In some instances, the foreign molecule is a cell-free nucleic acid derived from necrotic or apoptotic cells from tumor circulating within the subject's blood. Methods of evaluating whether a circulating nucleic acid derived from necrotic, apoptotic or normal tissue are provided herein in other sections. Such methods can also be used to detect or monitor necrotic or apoptotic tissue associated with cancer. For example, the method comprises detecting nucleic acids associated with necrotic tissue that are derived from cancerous tissue. In some instances, the method comprises detecting nucleic acids associated with apoptotic tissue that are derived from cancerous tissue. The method may further comprise predicting, evaluating, monitoring, diagnosing, or prognosing the existence of, stage of, or risk of, cancer in the subject based on the level of such nucleic acids (e.g., necrotic nucleic acids or apoptotic nucleic acids). The method may also comprise calculating a Death Mode Ratio, as described herein, to evaluate the relative level of apoptosis or necrosis. The method may also comprise comparing the level of nucleic acids associated with apoptotic tissue with either or both: (a) a healthy subject who does not have cancer and/or (b) a subject known to have cancer. Similarly, the method may comprise calculating a control Death Mode Ratio, as described further herein in other sections.

The method may further comprise evaluating circulating nucleic acids, such as those derived from cancerous cells, for certain hallmark characteristics of disease, such as cancer. This information can be used with, or in place of, information related to apoptosis or necrosis. For example, the circulating nucleic acids can be evaluated for one or more of the following signs of cancer: mutations in oncogenes, microsatellite alterations, and/or viral genomic sequences (which are relevant to cancers caused by viral pathogens such as the human papilloma virus).

Examples of tumor-associated circulating nucleic acids include, but are not limited to, those derived from prostate, breast, ovarian, uterine, cervical, lung, colon, uterine, pancreatic, bladder, brain, liver, kidney, and skin cancer. The disclosure further provides methods for monitoring response to radiation treatment and/or chemotherapeutic drugs, and monitoring cancer remission and recurrence.

In some cases, the heterogeneous samples are from a subject suffering from a cancer and heterogeneous sample comprises foreign molecules derived from a cancerous cell or tumor and molecules derived from a non-cancerous cell. The sample may comprise malignant tissue, benign tissue, or a mixture thereof. The cancer may be a recurrent and/or refractory cancer. Examples of cancers include, but are not limited to, sarcomas, carcinomas, lymphomas or leukemias.

Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavitity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

Detection of foreign molecules (e.g., cancer-derived molecules) in a subject suffering from cancer may be used in the diagnosis, prediction, or monitoring of a status or outcome of a cancer. For example, diagnosing, predicting, or monitoring a status or outcome of a cancer may comprise diagnosing or detecting a cancer, cancer metastasis, or stage of a cancer. In other instances, diagnosing, predicting, or monitoring a status or outcome of a cancer may comprise predicting the risk of cancer recurrence. In some cases, diagnosing, predicting, or monitoring a status or outcome of a cancer may comprise predicting mortality or morbidity. The methods provided herein may comprise treating a cancer or preventing a cancer progression. In addition, diagnosing, predicting, or monitoring a status or outcome of a cancer may comprise identifying or predicting responders to an anti-cancer therapy.

In some instances, the method comprises determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants, block cell division, and may also be used for the treatment of cancer. Alkaloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin and warfarin (coumadin, dicoumarol). Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase). Warfarin is a synthetic derivative of dicoumarol, a 4-hydroxycoumarin-derived mycotoxin anticoagulant.

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide. Alternatively, the anti-cancer agent comprises a proteasome inhibitor. Examples of proteasome inhibitors include bortezomib, disulfiram, epigallocatechin-3-gallage, salinosporamide A, carfilzomib, ONX912, CEP-18770, and MLN9708.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antiobiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose will be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed.

Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Brachytherapy may be used to treat patients with breast cancer who have undergone breast-conserving surgery.

The placement of brachytherapy sources can be temporary or permanent. For permament brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

b. Other Diseases, Disorders or Conditions

This disclosure also provides methods for detecting, monitoring, diagnosing and/or predicting diseases or disorders in a subject, including non-cancerous diseases or disorders. The methods provided herein may be particularly useful for detecting, monitoring, diagnosing and/or predicting diseases or disorders that are characterized by in an increased in cell death, including apoptotic and/or necrotic cell death. Examples of such diseases and disorders include but are not limited to: atherosclerosis, inflammatory diseases, autoimmune diseases, rheumatic heart disease. Examples of inflammatory diseases include, but are not limited to, acne vulgaris. Alzheimer's, ankylosing spondylitis, arthritis (osteoarthritis, rheumatoid arthritis (RA), psoriatic arthritis), asthma, atherosclerosis, celiac disease, chronic prostatitis, Crohn's disease, colitis, dermatitis, diverticulitis, fibromyalgia, glomerulonephritis, hepatitis, irritable bowel syndrome (IBS), systemic lupus erythematous (SLE), nephritis, Parkinson's disease, pelvic inflammatory disease, sarcoidosis, ulcerative colitis, and vasculitis. Examples of autoimmune diseases include, but are not limited to, acute disseminated encephalomyelitis (ADEM), Addison's disease, agammaglobulinemia, alopecia areata, amyotrophic Lateral Sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease/Balo concentric sclerosis, Behçet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's syndrome, cutaneous leukocytoclastic angiitis, Dego's diseasevDercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritisvepidermolysis bullosa acquisita, erythema nodosum, erythroblastosis fetalis, essential mixed cryoglobulinemia, Evan's syndrome, fibrodysplasia ossificans progressiva, fibrosing alveolitis (or idiopathic pulmonary fibrosis), gastritis, gastrointestinal pemphigoid, giant cell arteritis, glomerulonephritis, Goodpasture's syndrome, Graves' disease, Guillain-Barré syndrome (GBS), Hashimoto's encephalopathy, Hashimoto's thyroiditisvHenoch-Schonlein purpuravherpes gestationis aka gestational pemphigoid, hidradenitis suppurativa, Hughes-Stovin syndrome, hypogammaglobulinemia, idiopathic inflammatory demyelinating diseases, idiopathic pulmonary fibrosis, IgA nephropathy, inclusion body myositis, chronic inflammatory demyelinating polyneuropathyvinterstitial cystitis, juvenile idiopathic arthritis aka juvenile rheumatoid arthritis, Kawasaki's disease, Lambert-Eaton myasthenic syndrome, leukocytoclastic vasculitis, Lichen planus, Lichen sclerosus, linear IgA disease (LAD), Lou Gehrig's disease (Also Amyotrophic lateral sclerosis), lupoid hepatitis aka autoimmune hepatitis, lupus erythematosus, Majeed syndrome, Meniere's disease, microscopic polyangiitis, mixed connective tissue disease, morphea, Mucha-Habermann disease, multiple sclerosis, myasthenia gravis, myositis, neuromyelitis optica (also Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, paroxysmal nocturnal hemoglobinuria (PNH), Parry Romberg syndrome, Parsonage-Turner syndrome, Pars planitis, pemphigus vulgaris, pernicious anaemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, Schmidt syndrome another form of APS, Schnitzler syndrome, scleritis, scleroderma, serum sickness, Sjogren's syndrome, spondyloarthropathy, Stiff person syndrome, subacute bacterial endocarditis (SBE), Susac's syndrome, Sweet's syndrome, sympathetic ophthalmia, Takayasu's arteritis, temporal arteritis (also known as “giant cell arteritis”), thrombocytopenia, Tolosa-Hunt syndrome, transverse myelitis, ulcerative colitis, undifferentiated connective tissue disease different from mixed connective tissue disease, undifferentiated spondyloarthropathy, urticarial vasculitis, vasculitis, vitiligo, and Wegener's granulomatosis. The methods provided herein may also be useful for detecting, monitoring, diagnosing and/or predicting a subject's response to an implanted device. For example, the methods may comprise detecting dying or infected tissue near the implanted device using methods described herein. In another example, the methods may comprise detecting subject molecules (e.g., RNA, DNA, protein) from an immune cell (e.g., B-cell, T-cell, NK cell) using methods described herein. Exemplary medical devices include but are not limited to stents, replacement heart valves, implanted cerebella stimulators, hip replacement joints, breast implants, and knee implants.

Methods of evaluating whether a circulating nucleic acid derived from necrotic, apoptotic or normal tissue are provided herein in other sections. Such methods can also be used to detect or monitor necrotic or apoptotic tissue associated with diseases other than cancer or organ transplantation. For example, the method may comprise detecting nucleic acids associated with apoptotic tissue that may have derived from dying tissue related to a particular disease or condition. The method may further comprise predicting, evaluating, monitoring, diagnosing, or prognosing the existence of, stage of, or risk of, the disease or disorder in the subject based on the level of such nucleic acids. The method may also comprise calculating a Death Mode Ratio, as described herein, to evaluate the relative level of apoptosis or necrosis. The method may also comprise comparing the level of nucleic acids associated with apoptotic tissue with either or both: (a) a healthy subject who does not have the disease or disorder and/or (b) a subject known to have the disease or disorder. Similarly, the method may comprise calculating a control Death Mode Ratio, as described further herein in other sections.

c. Fetal Molecules

In some embodiments, the disclosure provides highly sensitive, non-invasive diagnostics for monitoring the health of a fetus using whole or partial genome analysis of nucleic acids derived from a fetus, as compared to the maternal genome. For example, circulating DNA can be useful in healthy patients for fetal diagnostics, with fetal DNA circulating in maternal blood serving as a marker for gender, rhesus D status, fetal aneuploidy, and sex-linked disorders. In some instances, the methods, compositions, and systems disclosed herein can replace more invasive and risky techniques such as amniocentesis or chorionic villus sampling. In some instances, the nucleic acids derived from the fetus are RNA molecules. Alternatively, the nucleic acids derived from the fetus are DNA molecules. The nucleic acids derived from the fetus can be cell-free nucleic acids. Alternatively, the nucleic acids derived from the fetus are from a cell. In some instances, a size profile of fetal molecules is used for fetal diagnostics. The size profile of the fetal molecules may be produced by any of the methods disclosed herein. The size profile of the fetal molecules can be indicative of apoptotic cell death. Alternatively, the size profile of the fetal molecules can be indicative of necrotic cell death.

The methods of the disclosure may involve analysis of mixed fetal and maternal nucleic acids (e.g., DNA, RNA) in the maternal blood to identify fetal mutations or genetic abnormalities from the background of maternal DNA. Differential detection of the fetal nucleic acid is achieved using whole genome sequencing to differentially detect and quantitate the genetic fingerprint of the fetus as compared to the maternal genome.

In a particular embodiment of the methods described herein, the starting material is maternal blood. In order to obtain sufficient DNA for testing, it is preferred that 10-20 mL of blood be drawn, in order to obtain at least 10,000 genome equivalents of total DNA. This sample size is based on an estimate of fetal DNA being present as roughly 25 genome equivalents/mL of maternal plasma in early pregnancy, and a fetal DNA concentration of about 3.4% of total plasma DNA. However, less blood may be drawn for a genetic screen in which less statistical significance is required, or in which the DNA sample is enriched for fetal DNA.

While the present description refers throughout to fetal DNA, fetal RNA found in maternal blood may be analyzed as well. As described in Ng et al., “mRNA of placental origin is readily detectable in maternal plasma,” Proc. Nat. Acad. Sci. 100(8): 4748-4753 (2003), hPL (human placental lactogen) and hCG (human chorionic gonadotropin) mRNA transcripts were detectable in maternal plasma, as analyzed using the respective real-time RT-PCR assays.

The maternal blood may be processed to enrich the fetal DNA concentration in the total DNA, as described in Li et al., supra. Briefly, circulatory DNA is extracted from 5 to 10 mL maternal plasma using commercial column technology (Roche High Pure Template DNA Purification Kit; Roche, Basel, Switzerland) in combination with a vacuum pump. After extraction, the DNA is separated by agarose gel (1%) electrophoresis (Invitrogen, Basel, Switzerland), and the gel fraction containing circulatory DNA with a size of approximately 300 bp is carefully excised. The DNA is extracted from this gel slice by using an extraction kit (QIAEX II Gel Extraction Kit; Qiagen, Basel, Switzerland) and eluted into a final volume of 40 μL sterile 10-mM trishydrochloric acid, pH 8.0 (Roche).

DNA and/or RNA may be concentrated by known methods, including centrifugation and various enzyme inhibitors. The DNA and/or RNA is bound to a selective membrane (e.g., silica) to separate it from contaminants. The DNA and/or RNA is preferably enriched for fragments circulating in the plasma, which are less than 1000 base pairs in length, generally less than 300 bp. This size selection is done on a DNA and/or RNA size separation medium, such as an electrophoretic gel or chromatography material. Such a material is described in Huber et al., “High-resolution liquid chromatography of DNA fragments on non-porous poly(styrene-divinylbenzene) particles,” Nucleic Acids Res. 1993 Mar. 11; 21(5): 1061-1066, gel filtration chromatography, TSK gel, as described in Kato et al., “A New Packing for Separation of DNA Restriction Fragments by High Performance Liquid Chromatography,” J. Biochem, 1984, Vol. 95, No. 1 83-86.

United States Patent Application 20040137470 also reports an enrichment procedure for fetal DNA. In this enrichment procedure, blood is collected into 9 ml EDTA Vacuette tubes (catalog number NC9897284), 0.225 ml of 10% neutral buffered solution containing formaldehyde (4% w/v) is added to each tube, and each tube gently is inverted. The tubes are stored at 4° C. until ready for processing.

Agents that impede cell lysis or stabilize cell membranes can be added to the tubes including but not limited to formaldehyde, and derivatives of formaldehyde, formalin, glutaraldehyde, and derivatives of glutaraldehyde, crosslinkers, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, cleavable crosslinkers, etc. Any concentration of agent that stabilizes cell membranes or impedes cell lysis can be added. In a preferred embodiment, the agent that stabilizes cell membranes or impedes cell lysis is added at a concentration that does not impede or hinder subsequent reactions.

Flow cytometry techniques can also be used to enrich fetal cells (Herzenberg et al., PNAS 76: 1453-1455 (1979); Bianchi et al., PNAS 87: 3279-3283 (1990); Bruch et al., Prenatal Diagnosis 11: 787-798 (1991)). U.S. Pat. No. 5,432,054 also describes a technique for separation of fetal nucleated red blood cells, using a tube having a wide top and a narrow, capillary bottom made of polyethylene. Centrifugation using a variable speed program results in a stacking of red blood cells in the capillary based on the density of the molecules. The density fraction containing low-density red blood cells, including fetal red blood cells, is recovered and then differentially hemolyzed to preferentially destroy maternal red blood cells. A density gradient in a hypertonic medium is used to separate red blood cells, now enriched in the fetal red blood cells from lymphocytes and ruptured maternal cells. The use of a hypertonic solution shrinks the red blood cells, which increases their density, and facilitates purification from the more dense lymphocytes. After the fetal cells have been isolated, fetal DNA and/or RNA can be purified using standard techniques in the art.

Further, an agent that stabilizes cell membranes may be added to the maternal blood to reduce maternal cell lysis including but not limited to aldehydes, urea formaldehyde, phenol formaldehyde, DMAE (dimethylaminoethanol), cholesterol, cholesterol derivatives, high concentrations of magnesium, vitamin E, and vitamin E derivatives, calcium, calcium gluconate, taurine, niacin, hydroxylamine derivatives, bimoclomol, sucrose, astaxanthin, glucose, amitriptyline, isomer A hopane tetral phenylacetate, isomer B hopane tetral phenylacetate, citicoline, inositol, vitamin B, vitamin B complex, cholesterol hemisuccinate, sorbitol, calcium, coenzyme Q, ubiquinone, vitamin K, vitamin K complex, menaquinone, zonegran, zinc, ginkgo biloba extract, diphenylhydantoin, perftoran, polyvinylpyrrolidone, phosphatidylserine, tegretol, PABA, disodium cromglycate, nedocromil sodium, phenyloin, zinc citrate, mexitil, dilantin, sodium hyaluronate, or polaxamer 188.

An example of a protocol for using this agent is as follows: The blood is stored at 4° C. until processing. The tubes are spun at 1000 rpm for ten minutes in a centrifuge with braking power set at zero. The tubes are spun a second time at 1000 rpm for ten minutes. The supernatant (the plasma) of each sample is transferred to a new tube and spun at 3000 rpm for ten minutes with the brake set at zero. The supernatant is transferred to a new tube and stored at −80° C. Approximately two milliliters of the “buffy coat,” which contains maternal cells, is placed into a separate tube and stored at −80° C.

In addition, enrichment may be accomplished by suppression of certain alleles through the use of peptide nucleic acids (PNAs), which bind to their complementary target sequences, but do not amplify.

Plasma RNA extraction is described in Enders et al., “The Concentration of Circulating Corticotropin-releasing Hormone mRNA in Maternal Plasma Is Increased in Preeclampsia,” Clinical Chemistry 49: 727-731, 2003. As described there, plasma harvested after centrifugation steps is mixed Trizol LS reagent (Invitrogen) and chloroform. The mixture is centrifuged, and the aqueous layer transferred to new tubes. Ethanol is added to the aqueous layer. The mixture is then applied to an RNeasy mini column (Qiagen) and processed according to the manufacturer's recommendations.

Detection of foreign molecules (e.g., fetal-derived DNA and/or RNA molecules) in a pregnant female may be used in the diagnosis, prediction, or monitoring of genetic abnormaity of the fetus. Examples of fetal genetic abnormalities include, but are not limited to, aneuploidy and other genetic variations, such as mutations, insertions, additions, deletions, translocations, inversions, point mutation, trinucleotide repeat disorders and/or single nucleotide polymorphisms (SNPs), as well as control targets not associated with fetal genetic abnormalities.

Often the methods and compositions described herein can enable detection of extra or missing chromosomes, particularly those typically associated with birth defects or miscarriage. For example, the diagnosis, prediction or monitoring of autosomal trisomies (e.g., Trisomy 13, 15, 16, 18, 21, or 22) may be based on the detection of foreign molecules. In some cases the trisomy may be associated with an increased chance of miscarriage (e.g., Trisomy 15, 16, or 22). In other cases, the trisomy that is detected is a liveborn trisomy that may indicate that an infant will be born with birth defects (e.g., Trisomy 13 (Patau Syndrome), Trisomy 18 (Edwards Syndrome), and Trisomy 21 (Down Syndrome)). The abnormality may also be of a sex chromosome (e.g., XXY (Klinefelter's Syndrome), XYY (Jacobs Syndrome), or XXX (Trisomy X). In certain preferred instances, the foreign molecule(s) to be detected is on one or more of the following chromosomes: 13, 18, 21, X, or Y. For example, the foreign molecule may be on chromosome 21 and/or on chromosome 18, and/or on chromosome 13. The foreign molecules may comprise multiple sites on multiple chromosomes.

Further fetal conditions that can be determined based on the methods and systems herein include monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g. XXXX, XXYY, XXXY, XYYY, XXXXX, XXXXY, XXXYY, XYYYY and XXYYY), monoploidy, triploidy (three of every chromosome, e.g. 69 chromosomes in humans), tetraploidy (four of every chromosome, e.g. 92 chromosomes in humans), pentaploidy and multiploidy.

In some cases, the genetic target comprises more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 225, 250, 300, 350, 400, 450, 500, 1,000, 5,000, 10,000, 20,000, 30,000, 40,000, 50,000, 60,000, 70,000, 80,000, 90,000 or 100,000 sites on a specific chromosome. In some cases, the genetic target comprises targets on more than 1, 2, 3, 4, 5, 6, 7, 8, 9, 10, 11, 12, 13, 14, 15, 16, 17, 18, 19, 20, 21, or 22 different chromosomes. In some cases the genetic target comprises targets on less than 2, 3, 4, 5, 6, 7, 8, 9, 10, 11, 12, 13, 14, 15, 16, 17, 18, 19, 20, 21, 22, or 23 chromosomes. In some cases, the genetic target comprises a gene that is known to be mutated in an inherited genetic disorder, including autosomal dominant and recessive disorders, and sex-linked dominant and recessive disorders. Non-limiting examples include genetic mutations that give rise to autoimmune diseases, neurodegenerative diseases, cancers, and metabolic disorders. In some instances, the method detects the presence of a genetic target associated with a genetic abnormality (such as trisomy), by comparing it in reference to a genetic target not associated with a genetic abnormality (such as a gene located on a normal diploid chromosome).

### V. General Therapeutic Diagnoses/Predictions/Regimens

In some instances, the methods, compositions, and systems disclosed here are used to diagnose a disease or condition in a subject. Diagnosing a disease or condition may comprise diagnosing a disease or condition such as cancer, pathogenic condition (e.g., viral infection, bacterial infection), transplant rejection, or genetic disorder. Diagnosing a disease or condition may comprise confirming a preliminary diagnosis. In some cases, diagnosing a disease or condition comprises determining the stage or level of severity of a disease, such as cancer; or otherwise classifying a disease. In another example, diagnosing a disease or condition comprises diagnosing a fetal genetic disorder in a fetus. In a particular embodiment, the methods of the invention provide the capability for sensitive, non-invasive, high throughput screening for diseases or conditions associated with the release of circulating nucleic acids into the bloodstream of a subject. The disease or condition may be a cancer, an organ transplant, a pathogenic infection, or pregnancy. The circulating nucleic acids are foreign nucleic acids and may be from a cancerous cell or tissue, a donor organ, a pathogen, or a fetus. In some instances, the circulating nucleic acids are host-derived nucleic acids and may be from a non-cancerous cell or a subject tissue, organ, or cell.

In other instances, the methods, compositions, and system disclosed herein are used to predict a status or outcome of a disease or condition. Predicting a status or outcome of a disease or condition may comprise predicting the risk of disease or injury. Predicting a status or outcome of a disease or condition may comprise predicting the risk of recurrence. Alternatively, predicting a status or outcome of a disease or condition may comprise predicting mortality or morbidity. In some instances, predicting a status or outcome of a disease or condition comprises identifying or predicting therapeutic responders. In other instances, predicting a status or outcome of a disease or condition comprises predicting risk of drug resistance.

In some instances, predicting a status or outcome of a disease or condition comprises predicting a transplant rejection or risk of a transplant rejection. Alternatively, predicting a status or outcome of a disease or condition comprises predicting the risk of cancer recurrence. Predicting a status or outcome of a disease or condition can also comprise predicting the risk of infection or injury. In some instances, predicting a status or outcome of a disease or condition comprises predicting an effectiveness of a therapeutic regimen or predicting a response to a therapeutic regimen.

Monitoring a status or outcome of a disease or condition may comprise preventing progression of the disease or condition. Alternatively, monitoring a status or outcome of a disease or condition comprises determining an efficacy of a therapeutic drug or regimen. The efficacy of a therapeutic regimen can be determined by detecting foreign molecules before, during and/or after a therapeutic drug or regimen is administered. In some instances, a decrease in foreign molecules is indicative of drug efficacy. For example, if the foreign molecules decrease by at least about 5%, at least about 10%, at least about 20%, at least about 30%, at least about 40%, at least about 50%, at least about 60%, at least about 70%, at least about 80%, at least about 90%, or at least about 95%, then the therapeutic regimen is deemed effective. Alternatively, if the foreign molecules decrease by at least about 20-fold, at least about 15-fold, at least about 10-fold, at least about 7-fold, at least about 5-fold, at least about 4-fold, at least about 3-fold, at least about 2-fold, at least about 1.5-fold, or at least about 1-fold, then the therapeutic regimen is deemed effective. In another example, if the foreign molecules comprise less than about 10%, less than about 7%, less than about 5%, less than about 4%, less than about 3%, less than about 2%, less than about 1.5%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% of the total molecules in the sample, than the drug is deemed effective. Alternatively, an increase in foreign molecules is indicative of drug failure, inefficiency, or refraction. For example, if the foreign molecules of increases by at least about 5%, at least about 10%, at least about 20%, at least about 30%, at least about 40%, at least about 50%, at least about 60%, at least about 70%, at least about 80%, at least about 90%, or at least about 95%, then the therapeutic regimen is deemed a failure or ineffective, or the subject is deemed refractory to the drug. Alternatively, if the foreign molecules increase by at least about 1-fold, at least about 1.5-fold, at least about 2-fold, at least about 3-fold, at least about 4-fold, at least about 5-fold, at least about 7-fold, at least about 10-fold, at least about 15-fold, or at least about 20-fold, then the therapeutic regimen is deemed a failure or ineffective, or the subject is deemed refractory to the drug. In another example, if the foreign molecules comprise greater than about 0.1%, greater than about 0.2%, greater than about 0.3%, greater than about 0.4%, greater than about 0.5%, greater than about 0.6%, greater than about 0.7%, greater than about 0.8%, greater than about 0.9%, greater than about 1%, greater than about 2%, greater than about 3%, greater than about 4%, greater than about 5%, greater than about 7%, greater than about 10%, greater than about 15%, or greater than about 20% of the total molecules in the sample, than the drug is deemed a failure or ineffective, or the subject is deemed refractory to the drug.

In some instances, the quantitative measurement of cell-free nucleic acids found within the various biological samples obtained from the subject, as described herein, is indicative of whether the therapeutic regimen is effective to treat a particular disease or condition (e.g., cancer, transplant rejection, pathogenic infection, or chimerism), or whether it needs to be adjusted to increase efficacy, or to avoid over-administration of harsh or harmful drugs or agents to the subject. In certain embodiments, the therapeutic regimen is increased if the percentage of cell-free nucleic acids from different genomic sources is greater than 1-2% of the total nucleic acids, preferably greater than or equal to 1% of the total nucleic acids, in the biological sample obtained from the subject. In other certain embodiments, the therapeutic regimen is decreased if the percentage of nucleic acids from different genomic sources is less than 1%, 0.9%, 0.8%, 0.7%, 0.6%, 0.5%, 0.4%, 0.3%, 0.2%, or 0.1% of the total nucleic acids in the biological sample.

In certain embodiments, the therapeutic drug is titrated and the percentage of cell-free nucleic acids (e.g., DNA, RNA) from different genomic sources is determined for a plurality of titration points. The percentage of cell-free nucleic acids for the plurality of titration points can be used to inform the proper dose of the therapeutic treatment regimen. The concentration endpoint may be specific to a particular organ, or particular individual, or both.

In some instances, the methods, compositions and systems disclosed herein are used to determine a treatment regimen. Determining a treatment regimen may comprise administering a drug (e.g., immunosuppressive therapy, anti-cancer drug, anti-microbial). Alternatively, determining a treatment regimen may comprise modifying, recommending, or initiating a therapeutic regimen. Modifying a therapeutic regimen comprises continuing, discontinuing, increasing, or decreasing a therapeutic regimen. In some instances, determining a treatment regimen comprises determining an optimal dose and/or optimal dosing schedule based on the presence or absence of foreign molecules. A therapeutic regimen may comprise one or more therapeutic drugs. The therapeutic regimen may comprise at least about 1, at least about 2, at least about 3, at least about 4, at least about 5, at least about 6, at least about 7, at least about 8, at least about 9, or at least about 10 therapeutic drugs.

In some instances, determining a therapeutic regimen comprises determining drug-specific baselines and/or thresholds. The drug-specific baselines and/or thresholds can provide ranges for modifying (e.g., adjusting, maintaining, initiating, or terminating) a therapeutic regimen. In some instances, modifying a therapeutic regimen may comprise increasing or decreasing a dosage of a therapeutic drug based on the presence or absence of foreign molecules. The dosage of therapeutic drug may be increased if the percentage of foreign molecules in the sample increases. The dosage of the therapeutic drug may be increased by at least about 2%, 5%, 7%, 10%, 15%, 20%, 30%, 40%, 50%, 60%, 70%, 80%, 90%, 95%, and 97%. Alternatively, the dosage of the therapeutic drug may be decreased if the percentage of foreign molecules in the sample decreases. The dosage of the therapeutic drug may be decreased by at least about 2%, 5%, 7%, 10%, 15%, 20%, 30%, 40%, 50%, 60%, 70%, 80%, 90%, 95%, and 97%.

In certain cases, the therapeutic regimen is modified if the percentage of nucleic acids from different genomic sources (e.g., foreign nucleic acids) is greater than about 1% of the total molecules (e.g., nucleic acids) in the biological sample obtained from the subject. In some instances, modifying a therapeutic regimen comprises increasing a therapeutic regimen. The therapeutic regimen may be increased if the percentage of nucleic acids from different genomic sources (e.g., foreign nucleic acids) is greater than about 0.1%, greater than about 0.2%, greater than about 0.3%, greater than about 0.4%, greater than about 0.5%, greater than about 0.6%, 0.7%, greater than about 0.8%, greater than about 0.9%, greater than about 1%, greater than about 2%, greater than about 3%, greater than about 4%, greater than about 5%, greater than about 6%, greater than about 7%, greater than about 8%, greater than about 9%, or greater than about 10% of the total molecules (e.g., nucleic acids) in the sample. Alternatively, if the percentage of foreign molecules is greater than about 0.1%, greater than about 0.2%, greater than about 0.3%, greater than about 0.4%, greater than about 0.5%, greater than about 0.6%, greater than about 0.7%, greater than about 0.8%, greater than about 0.9%, greater than about 1%, greater than about 2%, greater than about 3%, greater than about 4%, greater than about 5%, greater than about 6%, greater than about 7%, greater than about 8%, greater than about 9%, or greater than about 10% of the total molecules in the sample, then the therapeutic regimen is administered, increased, or initiated. Alternatively, if the percentage of foreign molecules is greater than about 0.1%, greater than about 0.2%, greater than about 0.3%, greater than about 0.4%, greater than about 0.5%, greater than about 0.6%, greater than about 0.7%, greater than about 0.8%, greater than about 0.9%, greater than about 1%, greater than about 2%, greater than about 3%, greater than about 4%, greater than about 5%, greater than about 6%, greater than about 7%, greater than about 8%, greater than about 9%, or greater than about 10% of the total molecules in the sample, then the frequency of dosage of the therapeutic drug is increased.

In other certain embodiments, the therapeutic regimen is modified if the percentage of nucleic acids from different genomic sources (e.g., foreign nucleic acids) is less than about 4%, 3%, 2%, 1%, 0.9%, 0.8%, 0.7%, 0.6%, 0.5%, 0.4%, 0.3%, 0.2%, or 0.1% of the total molecules (e.g., nucleic acids) in the biological sample obtained from the subject. In some instances, modifying a therapeutic regimen comprises decreasing a therapeutic regimen. The therapeutic regimen may be decreased if the percentage of nucleic acids from different genomic sources (e.g., foreign nucleic acids) is less than about 10%, less than about 7%, less than about 5%, less than about 3%, less than about 2%, less than about 1.5%, less than about 1%, less than about 0.7%, less than about 0.5%, or less than about 0.1% of the total molecules (e.g., nucleic acids) in the sample. Alternatively, if the percentage of foreign molecules is less than about 10%, less than about 7%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% of the total molecules (e.g, nucleic acids, DNA) in the sample, then the therapeutic regimen is decreased or terminated. Alternatively, if the percentage of foreign molecules is less than about 10%, less than about 7%, less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2%, or less than about 0.1% of the total molecules (e.g., nucleic acids, DNA) in the sample, then the frequency of dosage of the therapeutic drug is decreased.

In some instances, the foreign molecules increase by at least about 30%, 40%, 50%, 60%, 70%, 80%, 90%, 95%, or 100%. In other instances, the foreign molecules increase by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold. Alternatively, the foreign molecules increase by at least about 20-fold, at least about 30-fold, at least about 40-fold, at least about 50-fold, at least about 60-fold, at least about 70-fold, at least about 80-fold, at least about 90-fold, or at least about 100-fold.

In some instances, the foreign molecules decrease by at least about 30%, 40%, 50%, 60%, 70%, 80%, 90%, 95%, or 100%. In other instances, the foreign molecules decrease by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold. Alternatively, the foreign molecules decrease by at least about 20-fold, at least about 30-fold, at least about 40-fold, at least about 50-fold, at least about 60-fold, at least about 70-fold, at least about 80-fold, at least about 90-fold, or at least about 100-fold.

In some instances, determining a therapeutic regimen comprises administering a drug. The method may further comprise administering a test prior to administration of the drug. In some instances, the test comprises detecting the presence or absence of a foreign molecule. The test may comprise a diagnostic assay. For example, the test may comprise a viral detection test to diagnose a viral infection in a subject. Alternatively, the test comprises a bacterial detection test to diagnose a bacterial infection in a subject. In another instance, the test is a genetic test. For example, the subject is a pregnant female and the genetic test is administered to detect a fetal genetic abnormality. If a fetal genetic abnormality is detected, a drug may be administered to the subject.

In some instances, the methods, compositions, and systems disclosed herein are used as a companion diagnostic, whereby molecular assays that measure levels of proteins, nucleic acids, genes or specific mutations are used to provide a specific therapy for an individual's condition by stratifying disease status, selecting the proper medication and tailoring dosages to that patient's specific needs. Alternatively, such methods might be used to monitor the efficacy and/or toxicity of an immunosuppressive therapy administered to a subject suffering from a transplant rejection. The immunosuppressive therapy may be increased, decreased, or terminated based on the results of monitoring. In other instances, a new immunosuppressive therapy may be administered based on the results of the monitoring. Additionally, such methods might be used to assess a patient's risk factor for a number of conditions and tailor individual preventative treatments such as nutritional immunology approaches. Tissue-derived molecular information might be combined with an individual's personal medical history, family history, and data from imaging, and other laboratory tests to develop more effective treatments for a wider variety of conditions.

Determining a therapeutic regimen may comprise administering, modifying, initiating, or terminating a therapeutic regimen. Modifying a therapeutic regimen may comprise increasing or decreasing the dose of a therapeutic drug(s) and/or frequency of dosage of a therapeutic drug(s). Alternatively, modifying a therapeutic regimen can comprise adding or removing one or more therapeutic drugs. In some instances, determining a therapeutic regimen comprises determining the effective dose of a therapeutic drug. Alternatively, determining a therapeutic regimen comprises determining a dosing schedule for a therapeutic drug. The therapeutic regimen may be an anti-cancer regimen, immunosuppressive regimen, anti-pathogenic regimen (e.g., antibacterial, antiviral, antifungal). Alternatively, therapeutic regimen is a chemotherapeutic regimen, a radiation therapy regimen, a monoclonal antibody regimen, an anti-angiogenic regimen, an oligonucleotide therapeutic regimen, or any combination thereof. In some instances, the oligonucleotide therapeutic regimen comprises an antisense oligonucleotide, an miRNA, an siRNA, an aptamer or an RNA-based therapeutic. Often, the therapeutic regimens provided herein have better performance than standard regimens since circulating nucleic acids can serve as a better marker for the diagnosis, prediction, or monitoring of a status or outcome of a disease or condition as compared to standard markers (e.g., serum creatinine levels for kidney function or transaminase levels for liver function).

VI. Detection of molecules

Types of Molecules Detected

The methods disclosed herein often comprise conducting a reaction to detect a molecule (e.g., foreign molecule) in a heterogeneous sample from a subject. The method may further comprise detecting a molecule derived from a subject. The molecule (e.g., foreign molecule or subject-derived molecule) may be a biomolecule. Examples of biomolecules include, but are not limited to, a protein, a polypeptide, a peptide, a nucleic acid molecule, a nucleotide, an oligonucleotide, a polynucleotide, a saccharide, a polysaccharide, a cytokine, a growth factor, a morphogen, an antibody, a peptibody, or any fragment thereof. In some instances, the foreign molecule is a nucleic acid molecule or fragment thereof. Additionally, the subject molecule is a nucleic acid molecule or fragment thereof. The nucleic acid molecule may be a DNA molecule, RNA molecule (e.g. mRNA, cRNA or miRNA), and DNA/RNA hybrids. Examples of DNA molecules include, but are not limited to, double-stranded DNA, single-stranded DNA, single-stranded DNA hairpins, cDNA, genomic DNA. The nucleic acid may be an RNA molecule, such as a double-stranded RNA, single-stranded RNA, ncRNA, RNA hairpin, and mRNA. Examples of ncRNA include, but are not limited to, siRNA, miRNA, snoRNA, piRNA, tiRNA, PASR, TASR, aTASR, TSSa-RNA, snRNA, RE-RNA, uaRNA, x-ncRNA, hY RNA, usRNA, snaR, and vtRNA.

The DNA or RNA can be cell-free DNA or cell-free RNA. The DNA or RNA can be circulating, such as circulating within a bodily fluid (e.g., blood, urine). Circulating nucleic acids in bodily fluids such as blood can arise from necrotic or apoptotic cells. In some particular cases, the method comprises detecting or isolating cell-free RNA present in human plasma (Tong, Y.K. Lo, Y.M., Clin Chim Acta, 363, 187-196 (2006)) and cDNA sequencing of transcripts, thereby providing another option to detect circulating nucleic acids arising from foreign genomes.

In some instances, the foreign molecules are circulating or cell-free nucleic acids (e.g., cell-free DNA, cell-free RNA). In other instances, the subject molecules are circulating or cell-free nucleic acids (e.g., cell-free DNA, cell-free RNA).

As disclosed herein, the molecules may be from circulating foreign cells (e.g., donor cells, bacterial cells, virally infected cells, cancer cells). In some instances, the molecules are from circulating non-foreign cells. In other instances, the molecule is a circulating cell-free molecule. Alternatively, or additionally, the molecules are from an apoptotic cell or a necrotic cell. The molecules may be from a mitotic, post-mitotic, differentiated, redifferentiated, dedifferentiated, stem, pluripotent, or progenitor cell.

In some instances, the foreign molecule is a protein, polypeptide, peptide, or fragment thereof. Additionally, the subject molecule is a protein, polypeptide, peptide, or fragment thereof. Proteins, polypeptides, peptides may comprise cell surface markers (e.g., carbohydrates on bacterial cell walls, receptors), antibodies, transcription factors, translation factors, cell cycle regulators, enzymes, or kinases.

In some instances, the nucleic acid comprises a genetic variation such as a polymorphism. A polymorphism may comprise one or more base changes, an insertion, a repeat, or a deletion of one or more bases. Copy number variants (CNVs), transversions and other rearrangements are also forms of genetic variation. Polymorphic markers include single nucleotide polymorphisms (SNPs), restriction fragment length polymorphisms, variable number of tandem repeats (VNTRs), hypervariable regions, minisatellites, dinucleotide repeats, trinucleotide repeats, tetranucleotide repeats, simple sequence repeats, and insertion elements such as Alu.

The methods disclosed herein often involve conducting a reaction to detect a foreign molecule. In some instances, conducting a reaction to detect a molecule comprises detecting a foreign molecule such as a molecule derived from donor tissue that was transplanted into a subject. Alternatively, conducting a reaction to detect a molecule comprises detecting a molecule that is not a foreign molecule but is derived from a subject, such as a subject who is a transplant recipient. The reaction to detect the molecules may comprise dilution or distribution of a mixture of molecules in the biological sample into discrete sub-samples or individual molecules. For example, the reaction to detect the foreign molecules may comprise a digital PCR reaction. Alternatively, the reaction to detect the molecules does not comprise dilution or distribution of mixture of molecules in the biological sample into discrete sub-samples or individual molecules. For example, the reaction to detect the molecules may comprise direct sequencing of the foreign molecules in the sample comprising a plurality of molecules (e.g., foreign molecules and/or subject molecules).

In some instances, the methods, compositions and systems disclosed herein comprise the isolation of the molecule(s). By way of example only, nucleic acids or proteins are “isolated” when such nucleic acids or proteins are free of at least some of the cellular components with which it is associated in the natural state, or that the nucleic acid or protein has been concentrated to a level greater than the concentration of its in vivo or in vitro production. Nucleic acid can be isolated from the heterogeneous biological sample using techniques well known to those of ordinary skill in the art. See Sambrook, Fritsch and Maniatis, Molecular Cloning: A Laboratory Manual, 2nd edition (1989). In certain embodiments, genomic DNA is isolated from plasma using commercially available kits (e.g., Qiagen Midi Kit or QIAAmp Circulating Nucleic Acid Kit) for purification of DNA from blood cells, following the manufacturer's instructions (QIAmp DNA Blood Midi Kit, Catalog number 51183). DNA is eluted in 1001.11 of distilled water. The Qiagen Midi Kit can also be used to isolate DNA contained in the “buffy coat.” In some cases, conducting a reaction can comprise isolation of a foreign nucleic acid from a heterogeneous sample. The isolated foreign nucleic acid can be directly sequenced, thereby digitally separating the foreign genome from the host genome.

In some instances, conducting a reaction to detect a foreign molecule or subject molecule comprises generating a size profile of the molecules, sequencing the molecules, quantifying the molecules, or any combination thereof. For example, methods of the invention comprise the detection of fragments of molecules and conducting a size profile of the molecules. The methods of the invention may also comprise sequencing the foreign molecules. In another example, methods of the invention comprise the use of long-read sequencing technology. In some instances, long-sequencing read technology is used to digitally count whole genomes, or unique regions thereof, contained in a heterogeneous sample.

Detection Methods

Sequencing

Many different methods may be used to detect a molecule (e.g., subject molecule or foreign molecule) in a sample. In some instances, the molecules in a heterogeneous sample are detected by sequencing. The methods, compositions, and systems of the invention may comprise sequencing the foreign molecule (e.g., molecules from a pathogen, molecules from a transplanted organ or tissue, molecules from a cancerous cell or tissue, molecules from an unborn fetus). Additionally, subject molecules (e.g., molecules derived from an infected host, molecules derived from a transplant recipient, molecules derived from a non-cancerous cell or tissue, molecules derived from a pregnant female) are sequenced. Any technique for sequencing a nucleic acid known to those skilled in the art can be used in the methods of the provided invention. Sequencing may allow for the presence of multiple genotypes to be detected and quantified in a biological sample containing a mixture of genetic material from different genomic sources. Whole genomes, or unique regions thereof (e.g., genotype patterns such as variable number tandem repeats (VNTRs), short tandem repeats (STRs), and SNP patterns), can be detected and quantified.

In a particular embodiment, the nucleic acid is directly sequenced without diluting the genetic material within the sample or distributing the mixture of genetic material into discrete reaction samples. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Preferably, the sequencing technique used in the methods of the invention generates at least 100 reads per run, at least 200 reads per run, at least 300 reads per run, at least 400 reads per run, at least 500 reads per run, at least 600 reads per run, at least 700 reads per run, at least 800 reads per run, at least 900 reads per run, at least 1000 reads per run, at least 5,000 reads per run, at least 10,000 reads per run, at least 50,000 reads per run, at least 100,000 reads per run, at least 500,000 reads per run, or at least 1,000,000 reads per run. Alternatively, the sequencing technique used in the methods of the invention generates at least 1,500,000 reads per run, at least 2,000,000 reads per run, at least 2,500,000 reads per run, at least 3,000,000 reads per run, at least 3,500,000 reads per run, at least 4,000,000 reads per run, at least 4,500,000 reads per run, or at least 5,000,000 reads per run.

Preferably, the sequencing technique used in the methods of the invention can generate at least about 30 bp, at least about 40 bp, at least about 50 bp, at least about 60 bp, at least about 70 bp, at least about 80 bp, at least about 90 bp, at least about 100 bp, at least about 110, at least about 120 bp per read, at least about 150 bp, at least about 200 bp, at least about 250 bp, at least about 300 bp, at least about 350 bp, at least about 400 bp, at least about 450 bp, at least about 500 bp, at least about 550 bp, at least about 600 bp, at least about 700 bp, at least about 800 bp, at least about 900 bp, or at least about 1,000 bp per read. Alternatively, the sequencing technique used in the methods of the invention can generate long sequencing reads. In some instances, the sequencing technique used in the methods of the invention can generate at least about 1,200 bp per read, at least about 1,500 bp per read, at least about 1,800 bp per read, at least about 2,000 bp per read, at least about 2,500 bp per read, at least about 3,000 bp per read, at least about 3,500 bp per read, at least about 4,000 bp per read, at least about 4,500 bp per read, at least about 5,000 bp per read, at least about 6,000 bp per read, at least about 7,000 bp per read, at least about 8,000 bp per read, at least about 9,000 bp per read, or at least about 10,000 bp per read.

High-throughput sequencing systems may allow detection of a sequenced nucleotide immediately after or upon its incorporation into a growing strand, i.e., detection of sequence in real time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120, at least 150, at least 200, at least 250, at least 300, at least 350, at least 400, at least 450, or at least 500 bases per read. Sequencing can be performed using nucleic acids described herein such as genomic DNA, cDNA derived from RNA transcripts or RNA as a template.

Examples of high throughput sequencing methods include, but are not limited to, Lynx Therapeutics' Massively Parallel Signature Sequencing (MPSS), Polony sequencing, 454 pyrosequencing, Illumina (Solexa) sequencing, SOLiD sequencing, Ion Torrent™, Ion semiconductor sequencing, DNA nanoball sequencing, Helioscope™ single molecule sequencing, Single Molecule SMRT™ sequencing, Single Molecule real time (RNAP) sequencing, Nanopore DNA sequencing, and VisiGen Biotechnologies approach.

Suitable sequencing platforms that are useful with methods of the invention include, but are not limited to, True Single Molecule Sequencing (tSMS™) technology such as the HeliScope™ Sequencer offered by Helicos Inc. (Cambridge, Mass.), Single Molecule Real Time (SMRT™) technology, such as the PacBio RS system offered by Pacific Biosciences (California) and the Solexa Sequencer, Genome Analyzer IIx, HiSeq, and MiSeq offered by Illumina. In the tSMS technique, a DNA sample is cleaved into strands of approximately 100 to 200 nucleotides, and a polyA sequence is added to the 3′ end of each DNA strand. Helicos True Single Molecule Sequencing (tSMS) (Harris T. D. et al. (2008) Science 320:106-109). Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step.

Another example of a sequencing technology that can be used in the methods of the provided invention includes the single molecule, real-time (SMRT™) technology of Pacific Biosciences. In SMRT™, each of the four DNA bases is attached to one of four different fluorescent dyes. These dyes are phospholinked A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technology that can be used in the methods of the provided invention is SOLEXA sequencing (Illumina). SOLEXA sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a DNA sequencing technique that can be used in the methods of the provided invention is 454 sequencing (Roche) (Margulies, M et al. 2005, Nature, 437, 376-380). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the invention includes the Genome Sequencer FLX systems (Roche/454). The Genome Sequences FLX systems (e.g., GS FLX/FLX+, GS Junior) offer more than 1 million high-quality reads per run and read lengths of 400 bases. These systems are ideally suited for de novo sequencing of whole genomes and transcriptomes of any size, metagenomic characterization of complex samples, or resequencing studies.

SOLiD™ Sequencing is another example of a DNA sequencing technique that can be used in the methods. In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In some instances, sequencing comprises paired-end sequencing. Paired-end sequencing can comprise a modification to the standard single-read DNA library preparation, facilitating reading both the forward and reverse template strands of each cluster during one paired-end read. In addition to sequence information, both reads contain long-range positional information, allowing for highly precise alignment of reads and determination of molecule length. The Paired-End Sequencing Assay can utilize a combination of cBot (or the Cluster Station) and the Paired-End Module followed by paired-end sequencing on the Genome Analyzer_{IIx} or HiSeq or MiSeq. The paired-end sequencing protocol can also allow the end user to choose the length of the insert (200-500 bp) and sequence either end of the insert, generating highly quality, alignable sequence data. A typical paired-end run can achieve 2×150 bp reads and up to 200 million reads.

Nanopore sequencing is another example of a sequencing technique that can be used. A nanopore is a small hole, of the order of 1 nanometer in diameter Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA (for example, as described in US Patent Application Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope (Moudrianakis E. N. and Beer M. Proc Natl Acad Sci USA. 1965 March; 53:564-71). In one example of the technique, individual DNA molecules are labeled using metallic labels that are distinguishable using an electron microscope. These molecules are then stretched on a flat surface and imaged using an electron microscope to measure sequences.

In some instances, high-throughput sequencing involves the use of technology available by Helicos Biosciences Corporation (Cambridge, Mass.) such as the Single Molecule Sequencing by Synthesis (SMSS) method. SMSS is unique because it allows for sequencing the entire human genome with no pre-amplification step needed. Thus, distortion and nonlinearity in the measurement of nucleic acids are reduced. Sequencing methods may also allow for detection of a SNP nucleotide in a sequence in substantially real time or real time.

Alternatively, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

High-throughput sequencing may be performed using Clonal Single Molecule Array (Solexa, Inc.) or sequencing-by-synthesis (SBS) utilizing reversible terminator chemistry. High-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection.

Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. In general, high-throughput sequencing systems involve sequencing a target nucleic acid molecule having a plurality of bases by the temporal addition of bases via a polymerization reaction that is measured on a molecule of nucleic acid, e.g., the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable lo move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

Shotgun sequencing may be performed to detect molecules in a heterogeneous sample. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence

Sequencing techniques may also be used for detection and quantitation of SNPs. In this case, one can estimate the sensitivity of detection. There are two components to sensitivity: (i) the number of molecules analyzed (depth of sequencing) and (ii) the error rate of the sequencing process. Regarding the depth of sequencing, a frequent estimate for the variation between individuals is that about one base per thousand differs. Currently, sequencers such as the Illumina Genome Analyzer have read lengths exceeding 36 base pairs. Without intending to be limited to any theory or specific embodiment, this means that roughly one in 30 molecules analyzed will have a potential SNP. While the fraction of foreign nucleic acid molecules in a heterogeneous sample from a subject is currently not well determined and will depend on organ type, one can take 1% as a baseline estimate based on the literature and applicants own studies with heart transplant patients. At this fraction of foreign nucleic acid molecules, approximately one in 3,000 molecules analyzed will be from the foreign subject (e.g., donor, pathogen, cancer) and informative about donor genotype. On the Genome Analyzer one can obtain about 10 million molecules per analysis channel and there are 8 analysis channels per instrument run. Therefore, if one sample is loaded per channel, one should be able to detect about 3,000 molecules that can be identified as from the donor in origin, more than enough to make a precise determination of the fraction of donor DNA using the above parameters. If one wants to establish a lower limit of sensitivity for this method by requiring at least 100 donor molecules to be detected, then it should have a sensitivity capable of detecting donor molecules when the donor fraction is as low as 0.03%. Higher sensitivity can be achieved simply by sequencing more molecules, i.e. using more channels.

The sequencing error rate also affects the sensitivity of this technique. For an average error rate of ε, the chance of a single SNP being accidentally identified as of donor origin as a result of a misread is roughly ε/3. For each individual read, this establishes a lower limit of sensitivity of one's ability to determine whether the read is due to donor or recipient. Typical sequencing error rates for base substitutions vary between platforms, but are between 0.5-1.5%. This places a potential limit on sensitivity of 0.16 to 0.50%. However, it is possible to systematically lower the sequencing error rate by resequencing the sample template multiple times, as has been demonstrated by Helicos Biosciences (Harris, T. D., et al., Science, 320, 106-109 (2008)). A single application of resequencing would reduce the expected error rate of donor SNP detection to ε²/9 or less than 0.003%.

Genotyping

In some embodiments, the methods of the invention are used to genotype the donor and/or the recipient before transplantation to enable the detection of donor-specific nucleic acids such as DNA or RNA in bodily fluids such as blood or urine from the organ recipient after transplantation. Sequencing performed on the nucleic acid recovered from plasma or other biological samples may directly quantitate the percentage of donor-specific species within the sample. This approach allows for a reliable identification of sequences arising solely from the organ transplantation that can be made in a manner that is independent of the genders of donor and recipient.

The sequencing methods described herein are useful for generating a genetic fingerprint for the donor organ, tissue, or cell and/or a genetic fingerprint for the transplant recipient. Genotyping of transplant donors and transplant recipients prior to transplantation establishes a profile, using distinguishable markers, for detecting donor nucleic acids (e.g. circulating cell-free nucleic acid or nucleic acids from circulating donor cells). In some embodiments, for xenotransplants, nucleic acids from the donors can be mapped to the genome of the donor species.

Following transplantation, samples as described herein can be drawn from the patient and analyzed for the donor genetic fingerprint and/or the transplant recipient genetic fingerprint. The proportion of donor nucleic acids can be monitored over time and an increase in this proportion can be used to determine transplant status or outcome (e.g. transplant rejection).

In some embodiments, genotyping comprises whole genome sequencing and quantitation of nucleic acids from circulating transplant donor cells or circulating cell-free nucleic acids. In some embodiments, genotyping comprises detection and quantitation of polymorphic markers. Examples of polymorphic markers include single nucleotide polymorphisms (SNPs), restriction fragment length polymorphisms (RFLPs), variable number of tandem repeats (VNTRs), short tandem repeats (STRs), hypervariable regions, minisatellites, dinucleotide repeats, trinucleotide repeats, tetranucleotide repeats, simple sequence repeats, and insertion elements such as Alu. In some embodiments, genotyping comprises detection and quantitation of STRs. In some embodiments, genotyping comprises detection and quantitation of VNTRs.

In some instances, genotyping comprises genotyping foreign molecules. In some instances, genotyping comprises genotyping non-foreign molecules. In some instances, non-foreign molecules are genotyped and homozygous non-foreign positions are monitored for foreign bases. In some instances, the foreign molecules are not genotyped. In some embodiments, genotyping comprises detection and quantitation of SNPs. Without intending to be limited to any theory, any donor and recipient will vary at roughly three million SNP positions if fully genotyped. Usable SNPs must be homozygous for the recipient and ideally homozygous for the donor as well. While the majority of these positions will contain SNPs that are heterozygous for either the donor or the recipient, over 10% (or hundreds of thousands) will be homozygous for both donor and recipient meaning a direct read of that SNP position can distinguish donor DNA from recipient DNA. For example, after genotyping a transplant donor and transplant recipient, using existing genotyping platforms know in the art including the one described herein, one could identify approximately 1.2 million total variations between a transplant donor and transplant recipient. Usable SNPs may comprise approximately 500,000 heterozygous donor SNPs and approximately 160,000 homozygous donor SNPs.

Due to the low number of expected reads for any individual nucleic acid (e.g. SNP) in patient samples, some preamplification of the sample material may be required before analysis to increase signal levels, but using either preamplification, sampling more target nucleic acid positions (e.g. SNP positions), or both, will provide a reliable read-out of the transplant donor nucleic acid fraction. Preamplification can be performed using any suitable method known in the art such as multiple displacement amplification (MDA) (Gonzalez et al. Environ Microbiol; 7(7); 1024-8 (2005)) or amplification with outer primers in a nested PCR approach. This permits detection and analysis of donor nucleic acids even if the total amount of donor nucleic acid in the sample (e.g. blood from transplant patient) is only up to 1000 ng, 500 ng, 200 ng, 100 ng, 50 ng, 40 ng, 30 ng, 20 ng, 10 ng, 5 ng, 1 ng, 500 pg, 200 pg, 100 pg, 50 pg, 40 pg, 30 pg, 20 p, 10 pg, 5 pg, or 1 pg or between 1-5 μg, 5-10 μg, or 10-50 μg.

Methods, compositions and systems disclosed herein can provide for digital counting of whole genomes, or unique regions thereof. Methods, compositions and systems disclosed herein can afford more data via long sequence reads than traditional methods of analysis, such as PCR, SNP arrays, restriction fragment length polymorphism identification (RFLPI) of genomic DNA, random amplified polymorphic detection (RAPD) of genomic DNA, and amplified fragment length polymorphism detection (AFLPD). As such, methods of the invention can deliver a higher detection rate of circulating nucleic acids in a host subject and improved clinical performance compared to conventional screening methods.

Amplification

In some instances, the methods disclosed herein comprise amplification of the foreign molecules. Additionally, or alternatively, the methods disclosed herein comprise amplification of the subject-derived molecules. The RNA or DNA molecules may be cell-free. Alternatively, the RNA or DNA molecules are isolated from a cell. Amplification methods disclosed herein may be combined with reverse transcription to quantify and detect a foreign and/or subject-derived RNA molecule. In some instances, amplification comprises a PCR-based method. In some instances, the PCR-based method is PCR, quantitative PCR, emulsion PCR, droplet PCR, hot start PCR, in situ PCR, inverse PCR, multiplex PCR, Variable Number of Tandem Repeats (VNTR) PCR, asymmetric PCR, long PCR, nested PCR, hemi-nested PCR, touchdown PCR, assembly PCR, or colony PCR.

In other instances, amplification comprises a non-PCR-based method. In some instances, the non-PCR-based method is multiple displacement amplification (MDA), transcription-mediated amplification (TMA), nucleic acid sequence-based amplification (NASBA), strand displacement amplification (SDA), real-time SDA, rolling circle amplification, or circle-to-circle amplification.

Quantitative Methods

In some instances, the methods, compositions and systems disclosed herein comprise the use of quantitative methods for the detection of the molecules (e.g., DNA, RNA) in the sample. In some instances, the methods, compositions and systems disclosed herein comprise the use of quantitative methods for the detection of foreign molecules. Alternatively, or additionally, the methods, compositions, and systems disclosed herein comprise the use of quantitative methods for the detection of subject-derived molecules. Foreign molecules and subject-derived molecules may be RNA or DNA molecules. The RNA or DNA molecules may be cell-free. Alternatively, the RNA or DNA molecules are isolated from a cell. Examples of quantitative methods include, but are not limited to, qPCR, digital PCR, nanoreporters, and chromatography. Quantitative methods can be used to determine the percentage of molecules in a heterogeneous sample. The relative or absolute amounts of the molecules may also be determined by the quantitative methods disclosed herein.

Quantitative PCR (e.g., qPCR, RTQ-PCR) may be used to determine the amount of the foreign molecules in a heterogeneous sample. Alternatively, or additionally, quantitative PCR (e.g., qPCR, RTQ-PCR) may be used to determine the amount of subject-derived molecules in a heterogeneous sample. Generally, qPCR is used to amplify and simultaneously quantify a DNA molecule. In some instances, qPCR may be combined with reverse transcription to quantify and detect an RNA molecule. qPCR follows the same general principle of polymerase chain reaction, however, qPCR allows real time detection of the amplified molecule (e.g., as the reaction progresses, the amplified product may be detected). Two common methods for detection of products in qPCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target. qPCR can be used to quantify nucleic acids by two methods: relative quantification and absolute quantification. However, both relative and absolute quantification are comparative methods. Relative quantification is based on internal reference genes to determine fold-differences in expression of the target gene. Absolute quantification gives the exact number of target DNA molecules by comparison with DNA standards.

Digital PCR and/or next-generation sequencing methods allows for the presence of multiple genotypes to be detected and quantified in a biological sample containing a mixture of genetic material from different genomic sources. Partial or whole genomes, or unique regions thereof (e.g., genotype patterns such as variable number tandem repeats (VNTRs), short tandem repeats (STRs), and SNP patterns), can be detected and quantified.

Digital PCR (dPCR) may be used to directly quantify and clonally amplify nucleic acids including DNA, cDNA or RNA. dPCR also carries out a single reaction within a sample, however the sample is separated into a large number of partitions and the reaction is carried out in each partition individually. The partitioning of the sample allows one to count the molecules by estimating according to Poisson. As a result, each part will contain “0” or “1” molecules, or a negative or positive reaction, respectively. After PCR amplification, nucleic acids may be quantified by counting the regions that contain PCR end-product, positive reactions. In conventional PCR, starting copy number is proportional to the number of PCR amplification cycles. dPCR, however, is not dependent on the number of amplification cycles to determine the initial sample amount, eliminating the reliance on uncertain exponential data to quantify target nucleic acids and providing absolute quantification. Unlike qPCR, absolute quantification by dPCR is not a comparative method.

In some instances, quantitation of molecules comprises absolute quantitation. Absolute quantitation may comprise use of one or more control oligonucleotide species. The one or more control oligonucleotide species can be added to a sample from the subject. The one or more control oligonucleotide species can be of comparable size to the molecules expected in the cell-free molecules (e.g., DNA, RNA) samples. The one or more control oligonucleotide species can have distinct sequence identity. The one or more control oligonucleotide species can have non-natural sequence identity. The one or more control oligonucleotide species may comprise at least one deoxyribonucleotide and/or ribonucleotide. The one or more control oligonucleotide species can have one or more non-natural nucleotides. The samples with the control oligonucleotide species can be prepared and sequenced by any of the methods disclosed herein. The percentage of foreign molecules and the percentage of control oligonucleotide species can be calculated. The number of observed control oligonucleotide species can be used to calculate the number of molecules in the starting sample. Consequently, the absolute number of foreign molecules in the starting sample can be calculated. Control oligonucleotide species ratios and/or control oligonucleotide species lengths can be used to account for the biases resulting from different sample inputs. Alternatively, or additionally, the percentage of subject-derived molecules in the starting sample can be calculated.

Another method to quantify molecules may comprise the use of unique identifiers (e.g., barcodes, nanoreporters, labels). Generally, the molecules are labeled with unique identifiers and the uniquely labeled molecules may be detected by methods such as sequencing, ELISAs, and arrays and quantified. In some instances, the unique identifiers are nanoreporters, such as those described in U.S. Pat. No. 7,473,767, US publication number 2007/0166708, U.S. application Ser. No. 11/645,270, and PCT application number US06/049274. The unique identifiers may comprise nucleic acids, biomolecules, peptides, enzymes, kinases, proteins, antibodies, or antigens. The unique identifiers may be attached to the molecules and attachment may occur by ligation, binding, covalent attachment, hybridization or PCR. For example, a unique identifier comprising a nucleic acid may be ligated to a molecule (e.g., nucleic acid). In another example, a unique identifier comprising an antibody may be bound to a molecule (e.g., protein). Alternatively, the unique identifier is hybridized to the molecule.

In some instances, the molecule may be fragmented. Fragmentation of the molecules can occur by sonication, needle shear, nebulisation, shearing (e.g., acoustic shearing, mechanical shearing, point-sink shearing), passage through a French pressure cell, or enzymatic digestion. Enzymatic digestion may occur by nuclease digestion (e.g., micrococcal nuclease digestion, endonucleases, exonucleases, RNAse H or DNase I). Fragmentation of the target nucleic acid may result in fragment sized of about 100 bp to about 2000 bp, about 200 bp to about 1500 bp, about 200 bp to about 1000 bp, about 200 bp to about 500 bp, about 500 bp to about 1500 bp, and about 500 bp to about 1000 bp.

The detection, identification and/or quantitation of the molecules can be performed using arrays (e.g. SNP arrays). Results can be visualized using a scanner that enables the viewing of intensity of data collected and software to detect and quantify nucleic acid. Such methods are disclosed in part U.S. Pat. No. 6,505,125. Another method contemplated by the present invention to detect and quantify nucleic acids involves the use of beads as is commercially available by Illumina, Inc. (San Diego) and as described in U.S. Pat. Nos. 7,035,740; 7033,754; 7,025,935, 6,998,274; 6,942,968; 6,913,884; 6,890,764; 6,890,741; 6,858,394; 6,812,005; 6,770,441; 6,620,584; 6,544,732; 6,429,027; 6,396,995; 6,355,431 and US Publication Application Nos. 20060019258; 20050266432; 20050244870; 20050216207; 20050181394; 20050164246; 20040224353; 20040185482; 20030198573; 20030175773; 20030003490; 20020187515; and 20020177141; and in B. E. Stranger, et al., Public Library of Science-Genetics, I (6), December 2005; Jingli Cai, et al., Stem Cells, published online Nov. 17, 2005; C. M. Schwartz, et al., Stem Cells and Development, f 4, 517-534, 2005; Barnes, M., J. et al., Nucleic Acids Research, 33 (1 81, 5914-5923, October 2005; and Bibikova M, et al. Clinical Chemistry, Volume 50, No. 112, 2384-2386, December 2004. Additional description for preparing RNA for bead arrays is described in Kacharmina J E, et al., Methods Enzymol. 303: 3-18, 1999; Pabon C, et al., Biotechniques 3 1(4): 8769, 2001; Van Gelder R N, et al., Proc Natl Acad Sci USA 87: 1663-7 (1990); and Murray, S S. BMC Genetics B (SuppII):SX5 (2005).

When analyzing SNPs according to the methods described herein, the foreign and/or subject nucleic acids can be labeled and hybridized with a DNA microarray (e.g., 100K Set Array or other array). Results can be visualized using a scanner that enables the viewing of intensity of data collected and software “calls” the SNP present at each of the positions analyzed. Computer implemented methods for determining genotype using data mapping arrays are disclosed, for example, in Liu, et al., Bioinformatics 19:2397-2403, 2003; and Di et al., Bioinformatics 21: 1958-63, 2005. Computer implemented methods for linkage analysis using mapping array data are disclosed, for example, in Ruschendorf and Nusnberg, Bioinformatics 21:2123-5, 2005; and Leykin et al., BMC Genet. 6:7, 2005; and in U.S. Pat. No. 5,733,729.

In some instances, genotyping microarrays that are used to detect SNPs can be used in combination with molecular inversion probes (MIPs) as described in Hardenbol et al., Genome Res. 15(2):269-275, 2005; Hardenbol, P. et al. Nature Biotechnology 21(6), 673-8, 2003; Faham M, et al. Hum Mol Genet. 10(16):1657-64, 2001; Maneesh Jain, Ph.D., et al. Genetic Engineering News V24: No. 18, 2004; Fakhrai-Rad H, et al. Genome Res. July; 14(7): 1404-12, 2004; and in U.S. Pat. No. 5,858,412. Universal tag arrays and reagent kits for performing such locus specific genotyping using panels of custom M1Ps are available from Affymetrix and Par Allele. MIP technology involves the use of enzymological reactions that can score up to 10,000; 20,000; 50,000; 100,000; 200,000; 500,000; 1,000,000; 2,000,000 or 5,000,000 SNPs (target nucleic acids) in a single assay. The enzymological reactions are insensitive to cross-reactivity among multiple probe molecules and there is no need for pre-amplification prior to hybridization of the probe with the genomic DNA. In some instances, the molecules or SNPs can be obtained from a single cell.

Electrophoretic methods may also be used to detect and/or quantify molecules. In some instances, the methods, compositions and systems disclosed herein comprise electrophoretic detection and/or quantitation of foreign molecules. Alternatively, or additionally, the methods, compositions and systems disclosed herein comprise electrophoretic detection and/or quantitation of subject-derived molecules. For example, gel electrophoresis is used to detect nucleic acids and proteins and includes overlay gel electrophoresis, charge shift method, band shift assay, countermigration electrophoresis, affinophoresis, affinity electrophoresis, rocket immunoelectrophoresis, and crossed immunoelectrophoresis. Another example of an electrophoretic method is capillary electrophoresis. Affinity capillary electrophoresis has demonstrated its value in the measurement of binding constants, the estimation of kinetic rate constants, and the determination of stoichiometry of biomolecular interactions. It offers short analysis time, requires minute amounts of protein samples, usually involves no radiolabeled compounds, and, most importantly, is carried out in solution. SDS-PAGE may also be used to detect nucleic acid molecules. Electrophoretic methods may also be used to generate a size profile of the molecules, thereby detecting foreign molecules.

Additional methods for quantifying molecules include, but are not limited to, gas chromatography, supercritical fluid chromatography, liquid chromatography (including partition chromatography, adsorption chromatography, ion exchange chromatography, size exclusion chromatography, thin-layer chromatography, and affinity chromatography), electrophoresis (including capillary electrophoresis, capillary zone electrophoresis, capillary isoelectric focusing, capillary electrochromatography, micellar electrokinetic capillary chromatography, isotachophoresis, transient isotachophoresis and capillary gel electrophoresis), comparative genomic hybridization (CGH), microarrays, bead arrays, and high-throughput genotyping such as with the use of molecular inversion probe (MIP). Southern blot and Northern blot may also be used to detect and/or quantify nucleic acid molecules. ELISA, immunofluorescence, and Western blot are additional methods that may be used to detect and/or quantify biomolecules (e.g., proteins).

The quantification method may involve amplification; although, in some cases, the method may be amplification independent. Cylindrical illumination confocal spectroscopy and molecular barcoding are examples of quantification methods that can be conducted in an amplification-independent manner.

Alternatively, fluorescent dyes may also be used for the detection and/or quantification of molecules. Fluorescent dyes may typically be divided into families, such as fluorescein and its derivatives; rhodamine and its derivatives; cyanine and its derivatives; coumarin and its derivatives; Cascade Blue™ and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa Fluor®-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX™), LIZ™, VIC™, NED™, PET™, SYBR, PicoGreen, RiboGreen, and the like. Descriptions of fluorophores and their use, can be found in, among other places, R. Haugland, Handbook of Fluorescent Probes and Research Products, 9.sup.th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

In another aspect of the invention, a branched-DNA (bDNA) approach is used to increase the detection sensitivity. In some instances, bDNA approach is applied to an array detection assay. The array detection assay can be any array assay known in the art, including the array assays described herein. bDNA approach amplifies the signals through a branched DNA that are attached by tens or hundreds of alkaline phosphatase molecules. Thus, the signals are significantly amplified while the fidelity of the original nucleic acid target abundance is maintained.

Marker Profile

In some instances, detection of the molecules may comprise producing a marker profile. The marker profile may comprise one or more molecules or fragments thereof. In some instances, producing a marker profile comprises sequencing the foreign molecules. The foreign molecules may be DNA or RNA molecules. The DNA or RNA molecules may be from a cell. Alternatively, the DNA or RNA molecules are cell-free molecules. The marker profile may comprise at least about 1, at least about 2, at least about 3, at least about 4, at least about 5, at least about 10, at least about 15, at least about 20, at least about 30, at least about 40, at least about 50, at least about 100, at least about 200, at least about 300, at least about 400, at least about 500, at least about 600, at least about 700, at least about 800, at least about 900, or at least about 1000 foreign molecules. The foreign molecules may be identical, similar, or different. Identical foreign molecules may comprise the same sequence (nucleotide or peptide sequence). In some instances, the sequence of two or more foreign molecules in the marker profile are at least about 10%, 20%, 30%, 40%, 50%, 60%, 70%, 75%, 80%, 90%, 95%, or 97% identical. The sequence of two or more foreign may overlap (partially or fully). The sequence of two or more foreign molecules may be different. In some instances, the sequence of two or more foreign molecules may be less than about 1%, 5%, 10%, 15%, 20%, 25%, 30%, 35%, 40%, 50%, 60%, 70%, 75%, 80%, 90%, 95%, or 97% identical.

Alternatively, or additionally, producing a marker profile comprises sequencing the subject molecules. The subject molecules may be DNA or RNA molecules. The DNA or RNA molecules may be from a cell. Alternatively, the DNA or RNA molecules are cell-free molecules. The marker profile may comprise at least about 1, at least about 2, at least about 3, at least about 4, at least about 5, at least about 10, at least about 15, at least about 20, at least about 30, at least about 40, at least about 50, at least about 100, at least about 200, at least about 300, at least about 400, at least about 500, at least about 600, at least about 700, at least about 800, at least about 900, or at least about 1000 subject molecules.

The methods disclosed herein may comprise the detection of at least about 10%, 20%, 30%, 40%, 50%, 60%, 70%, 80%, 90%, 95%, or 97% of the foreign molecules in the heterogeneous sample. In some instances, the foreign molecules comprise less than about 90%, 80%, 70%, 60%, 50%, 40%, 30%, or 20% of the total molecules in the sample. Preferably, the foreign molecules comprise less than about 15%, 10%, 9%, 8%, 7%, 6%, 5%, 4%, 3%, 2%, or 1% of the total molecules in the sample. In some instances, the foreign molecules comprise less than about 1%, 0.5%, 0.25%, 0.1% of the total molecules in the sample.

The methods disclosed herein may comprise the detection of at least about 10%, 20%, 30%, 40%, 50%, 60%, 70%, 80%, 90%, 95%, or 97% of the subject molecules in the heterogeneous sample. In some instances, the subject molecules comprise less than about 90%, 80%, 70%, 60%, 50%, 40%, 30%, or 20% of the total molecules in the sample. Alternatively, the subject molecules may comprise less than about 15%, 10%, 9%, 8%, 7%, 6%, 5%, 4%, 3%, 2%, or 1% of the total molecules in the sample. In some instances, the subject molecules comprise greater than about 95%, 96%, 97%, 98%, 98.5%, 99%, 99.5%, or 99.9% of the total molecules in the sample.

Size Profile

In some instances, the methods disclosed herein comprise generating a size profile of the molecules. Generating a size profile may comprise electrophoretic separation of the molecules and/or sequencing the molecules. The size profile may comprise nucleic acid fragments (e.g., DNA fragments, RNA fragments). In some instances, the nucleic acid fragments comprise DNA fragments. Alternatively, the nucleic acid fragments comprise RNA fragments. In some instances, the nucleic acid fragments are cell-free nucleic acid fragments. Alternatively, the nucleic acid fragments are from a cell. The nucleic acid fragments may be foreign molecules. The nucleic acid molecules may be subject-derived molecules. The nucleic acid fragments may form a ladder of sizes. In some instances, the ladder of sizes comprises nucleic acid fragments of about 180 bp increments. For example, the ladder may comprise of nucleic acid fragments of about 180 bp, about 360 bp, about 540 bp, about 720 bp, about 900 bp, about 1080 bp, about 1260 bp, about 1440 bp, about 1620 bp, about 1800 bp, etc.

In some instances, for a urine sample, a different use of sizing may involve filtering DNA from the urinary system from DNA coming from other parts of the body (e.g., an organ such as a heart, lung, or liver). Healthy kidneys can normally function to filter out DNA from the blood, though, in some instances, small DNA fragments pass through the kidney. Donor-derived signal can be enriched by isolating sequences, either experimentally or informatically, that are less than about 25, 50, 75, 100, 125, or 150 base pairs. Similarly, kidney-derived or other urinary tract DNA could be enriched by selecting molecules that are at least about 150, 200, 300, or 500 base pairs in length. In some embodiments, the use of both small and/or large DNA fragments may be used to inform patient treatment. For example, the amount of donor DNA observed in small fragments reveals that the donor graft is healthy, thereby resulting in a reduction in an immunosuppressive therapy. In another example, an increase in kidney-derived DNA is indicative of drug nephrotoxicity, thereby resulting in a reduction in an immunosuppressive therapy and/or administration of a new immunosuppressive therapy.

In some instances, the size distribution of the foreign molecules is assayed by sequencing. In addition, the subject-derived molecules are also assayed by sequencing. Sequencing, such as paired-end sequencing, can be performed on the molecules. Identification of subject and foreign-specific SNPs can be used to identify all foreign-derived molecules. The size of the foreign molecules can be calculated from the paired-end sequence alignment. A histogram of sizes can be made. In some instances, features of the size distribution, including mean/median size and the extent of apoptotic ladder-like patterning, is used to determine the ratio of apoptotic versus necrotic contribution. The size distribution and/or the ratio of apoptotic versus necrotic contribution can be used to perform differential diagnosis of different causes of graft injury. Optionally, overall foreign molecule levels, sequences from an infectious agent, and/or sequences from an immune repertoire are also used to perform differential diagnosis of different causes of graft injury.

In some instances, the size profile comprises at least about 1, at least about 2, at least about 3, at least about 4, at least about 5, at least about 10, at least about 15, at least about 20, at least about 25, at least about 30, at least about 35, at least about 40, at least about 45, at least about 50, at least about 60, at least about 70, at least about 80, at least about 90, or at least about 100 molecules. Alternatively, the size profile comprises at least about 500, at least about 1,000, at least about 2,000, at least about 3,000, at least about 4,000, at least about 5,000, at least about 6000, at least about 7,000, at least about 8,000, at least about 9,000, at least about 10,000, at least about 15,000, at least about 20,000, at least about 25,000, at least about 30,000, at least about 40,000, at least about 50,000, at least about 75,000, or at least about 100,000 molecules. Additional information about size profiles can be found in other sections of the present disclosure, such as sections relating to organ transplantation. Such size profiles can also be used for diseases or conditions outside of the organ transplantation setting.

Analysis

After digitally counting the number of genomes and/or genotype patterns present in the heterogeneous biological sample, ratios of the different genomes and/or genotype patterns can then be compared to determine the relative amounts of the various genotypes and/or genotype patterns in the biological sample. By counting the number of genomes and/or genotype patterns, the over- or underrepresentation of any foreign genome within a given individual can be detected. It should be noted that methods of the invention do not require the differentiation of foreign versus host nucleic acid, and with large enough sequence counts, methods of the invention can be applied to arbitrarily small fractions of foreign nucleic acid.

High-throughput analysis can be achieved using one or more bioinformatics tools, such as ALLPATHS (a whole genome shotgun assembler that can generate high quality assemblies from short reads), Arachne (a tool for assembling genome sequences from whole genome shotgun reads, mostly in forward and reverse pairs obtained by sequencing cloned ends, BACCardl (a graphical tool for the validation of genomic assemblies, assisting genome finishing and intergenome comparison), CCRaVAT & QuTie (enables analysis of rare variants in large-scale case control and quantitative trait association studies), CNV-seq (a method to detect copy number variation using high throughput sequencing), Elvira (a set of tools/procedures for high throughput assembly of small genomes (e.g., viruses)), Glimmer (a system for finding genes in microbial DNA, especially the genomes of bacteria, archaea and viruses), gnumap (a program designed to accurately map sequence data obtained from nextgeneration sequencing machines), Goseq (an R library for performing Gene Ontology and other category based tests on RNA-seq data which corrects for selection bias), ICAtools (a set of programs useful for medium to large scale sequencing projects), LOCAS (a program for assembling short reads of second generation sequencing technology), Maq (builds assembly by mapping short reads to reference sequences, MEME (motif-based sequence analysis tools, NGSView (allows for visualization and manipulation of millions of sequences simultaneously on a desktop computer, through a graphical interface, OSLay (Optimal Syntenic Layout of Unfinished Assemblies), Perm (efficient mapping for short sequencing reads with periodic full sensitive spaced seeds, Projector (automatic contig mapping for gap closure purposes), Qpalma (an alignment tool targeted to align spliced reads produced by sequencing platforms such as Illumina, Solexa, or 454), RazerS (fast read mapping with sensitivity control), SHARCGS (SHort read Assembler based on Robust Contig extension for Genome Sequencing; a DNA assembly program designed for de novo assembly of 25-40 mer input fragments and deep sequence coverage), Tablet (next generation sequence assembly visualization), and Velvet (sequence assembler for very short reads).

The methods described herein are used to detect and/or quantify whole genomes or genomic DNA regions. In some embodiments, the methods described herein can discriminate and quantitate genomic DNA regions. The methods described herein can discriminate and quantitate at least 1; 2; 3; 4; 5; 10, 20; 50; 100; 200; 500; 1,000; 2,000; 5,000; 10,000, 20,000; 50,000; 100,000; 200,000; 300,000; 400,000; 500,000; 600,000; 700,000; 800,000; 900,000; 1,000,000; 2,000,000 or 3,000,000 different genomic DNA regions. The methods described herein can discriminate and quantitate genomic DNA regions varying by 1 nt or more than 1, 2, 3, 5, 10, 15, 20, 21, 22, 24, 25, 30, 40, 50, 60, 70, 80, 90, 100, 200, 300, 400 or 500 nt.

In some cases, the methods described herein are used to detect and/or quantify genomic DNA regions such as a region containing a DNA polymorphism. A polymorphism refers to the occurrence of two or more genetically determined alternative sequences or alleles in a population. A polymorphic marker or site is the locus at which divergence occurs. Preferred markers have at least two alleles, each occurring at a frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include single nucleotide polymorphisms (SNPs), restriction fragment length polymorphisms (RFLPs), short tandem repeats (STRs), variable number of tandem repeats (VNTRs), hypervariable regions, minisatellites, dinucleotide repeats, trinucleotide repeats, tetranucleotide repeats, simple sequence repeats, and insertion elements such as Alu. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

In some cases, the methods described herein can discriminate and quantitate a DNA region containing a DNA polymorphism. The methods described herein can discriminate and quantitate of at least 1; 2; 3; 4; 5; 10, 20; 50; 100; 200; 500; 1,000; 2,000; 5,000; 10,000, 20,000; 50,000; 100,000; 200,000; 300,000; 400,000; 500,000; 600,000; 700,000; 800,000; 900,000; 1,000,000; 2,000,000 or 3,000,000 DNA polymorphism. In some embodiments, the methods described herein can discriminate and quantitate at least 1; 2; 3; 4; 5; 10; 20; 50; 100; 200; 500; 1,000; 2,000; 5,000; 10,000; 20,000; 50,000; 100,000; 200,000; 300,000; 400,000; 500,000; 600,000; 700,000; 800,000; 900,000; 1,000,000; 2,000,000 or 3,000,000 different polymorphic markers. In some embodiments, the methods described herein can discriminate and quantitate at least 1; 2; 3; 4; 5; 10; 20; 50; 100; 200; 500; 1,000; 2,000; 5,000; 10,000; 20,000; 50,000; 100,000; 200,000; 300,000; 400,000; 500,000; 600,000; 700,000; 800,000; 900,000; 1,000,000; 2,000,000 or 3,000,000 different SNPs.

In some cases, the methods described herein are used to detect and/or quantify gene expression. In some embodiments, the methods described herein provide high discriminative and quantitative analysis of multiple genes. The methods described herein can discriminate and quantitate the expression of at least 1, 2, 3, 4, 5, 10, 20, 50, 100, 200, 500, 1,000, 2,000, 5,000, 10,000, 20,000, 50,000, 100,000, different target nucleic acids. In some instances, the target nucleic acids are DNA molecules. The DNA molecules may comprise introns and/or exons. The DNA molecules may be cDNA molecules. Alternatively, the target nucleic acids are RNA molecules. In some instances, the RNA molecules are mRNA molecules. The mRNA molecules may be immature mRNA molecules. Alternatively, the mRNA molecules are mature mRNA molecules. The DNA and/or RNA molecules may be from a subject. Alternatively, or additionally, the DNA and/or RNA molecules may be from a foreign source.

Gene expression of one or more DNA and/or RNA molecules can be used to determine the health of particular cells, tissues, or organs. For example, some genes may only be expressed, or may be primarily expressed, in heart tissue, and the quantification of RNA from these genes would give a signal regarding the status of the heart. In some instances, the cell, tissue or organ is from a transplant donor. Alternatively, the cell, tissue, or organ is from the subject. Gene expression of one or more DNA and/or RNA molecules can be used to determine the health of a subject. For example, gene expression of one or more DNA and/or RNA molecules from a cancerous cell can be used to determine the health of a subject suffering from a cancer. In another example, gene expression of one or more DNA and/or RNA molecules from a pathogen and/or pathogen-infected subject can be used to determine the health of a subject suffering from a pathogenic infection. Alternatively, gene expression of one or more DNA and/or RNA molecules can be used to determine the health of a fetus. The signal may comprise the presence/absence of RNAs from a particular gene or several genes. The signal may also represent an increase, or decrease, in the level of a particular gene or several genes.

In some embodiments, the methods described herein are used to detect and/or quantify gene expression of genes with similar sequences. The methods described herein can discriminate and quantitate the expression of genes varying by 1 nt or more than 1, 2, 3, 5, 10, 15, 20, 21, 22, 24, 25, 30, 40, 50, 60, 70, 80, 90, 100, 200, 300, 400 or 500 nt.

In some embodiments, the methods described herein are used to detect and/or quantify genomic DNA regions by mapping the region to the genome of a species in the case where the transplant donor and the transplant recipient are not from the same species (e.g., xenotransplants). In some embodiments, the methods described herein can discriminate and quantitate a DNA region from a species. The methods described herein can discriminate and quantitate of at least 1; 2; 3; 4; 5; 10, 20; 50; 100; 200; 500; 1,000; 2,000; 5,000; 10,000, 20,000; 50,000; 100,000; 200,000; 300,000; 400,000; 500,000; 600,000; 700,000; 800,000; 900,000; 1,000,000; 2,000,000 or 3,000,000 DNA regions from a species.

In some instances, the foreign molecules are detected in a multiplexed reaction. For example, at least about 2, at least about 3, at least about 4, at least about 5, at least about 10, at least about 15, at least about 20, at least about 30, at least about 40, at least about 50, at least about 100, at least about 200, at least about 300, at least about 400, at least about 500, at least about 600, at least about 700, at least about 800, at least about 900, or at least about 1000 molecules are detected in a single reaction or a single reaction container. In another example, at least about 2000, at least about 5000, at least about 10000, at least about 15000, at least about 20000, at least about 30000, at least about 40000, at least about 50000, at least about 100000, at least about 200000, at least about 300000, at least about 400000, at least about 500000, at least about 600000, at least about 700000, at least about 800000, at least about 900000, or at least about 1000000 molecules are detected in a single reaction or a single reaction container.

Detection of the foreign molecules may comprise the detection of genetic variants. In some instances, at least about 2, at least about 3, at least about 4, at least about 5, at least about 10, at least about 15, at least about 20, at least about 30, at least about 40, at least about 50, at least about 100, at least about 200, at least about 300, at least about 400, at least about 500, at least about 600, at least about 700, at least about 800, at least about 900, or at least about 1000 genetic variants are detected in a single reaction. In another example, at least about 2000, at least about 5000, at least about 10000, at least about 15000, at least about 20000, at least about 30000, at least about 40000, at least about 50000, at least about 100000, at least about 200000, at least about 300000, at least about 400000, at least about 500000, at least about 600000, at least about 700000, at least about 800000, at least about 900000, or at least about 1000000 genetic variants are detected in a single reaction.

In some instances, detection of molecules comprises physically separating the molecules into a single target molecule in a reaction. Alternatively, the detection of molecules does not comprise dilution or distribution of the target molecules in the sample into discrete sub-samples or individual molecules. Often, detection of the target molecules occurs in a single reaction volume. The target molecules can be detected simultaneously or sequentially. In some instances, target molecules derived from a foreign genotype are detected. Alternatively, target molecules from multiple genotypes are detected. For example, foreign molecules and subject molecules are detected.

In some instances, the presence or absence of molecules is determined by the use of an integral detector. Generally, an integral detector measures the accumulated quantity of sample component(s) that reach the detector. Alternatively, the presence or absence of molecules is determined by the use of a differential detector. Generally, differential detection is an encoding and detection technique that uses phase changes in the carrier to signal binary ones and zeros.

### VII. Heterogeneous Samples

The present disclosure provides methods and compositions for the detection of foreign molecules within a heterogeneous sample (e.g., a sample comprising at least two different genomic sources). Heterogeneous samples may be from a transplant recipient, a chimeric individual, a subject suffering from cancer, a subject suffering from a disease or condition caused by a pathogen, or a subject with a different disease, disorder or condition.

The heterogeneous sample may be from a tissue, organ, or bodily fluid of a subject. The heterogeneous biological sample can be blood, a blood fraction (e.g., plasma, serum), saliva, sputum, urine, semen, transvaginal fluid, vaginal flow, cerebrospinal fluid, brain fluid, sweat, breast milk, breast fluid (e.g., breast nipple aspirate), stool, bile, secretions, lymph fluid, tears, ear flow, lymph, bone marrow suspension, or ascites. Preferably, the sample is from blood. In some instances, the biological sample is from whole blood, plasma, or serum. In some cases, the biological sample is urine.

In some cases, the heterogeneous biological sample is derived from secretions of the respiratory, intestinal or genitourinary tracts or from a lavage of a tissue or organ (e.g. lung) or tissue which has been removed from organs. The heterogeneous biological sample may be a cell or a tissue biopsy, or a sample taken as a smear. In a particular embodiment, the biological sample is drawn blood and circulating nucleic acids (or other molecules such as proteins) from different genomic sources is found in the blood or plasma, rather than in cells.

The heterogeneous sample can comprise a mixture of molecules (e.g., genetic material, nucleic acids, proteins) from at least two different genomic sources. The different genomic sources contributing the genetic material or other molecules to the biological sample can be from any of the following sets of sources: a pregnant female and a fetus; a non-cancerous cell or a tissue and a cancerous cell or tissue; a donor tissue and a transplant recipient tissue; a healthy cell or tissue and diseased cell or tissue; or from a pathogen (e.g, virus, bacterium, fungus) and an infected subject.

In some cases, the heterogeneous samples are from a chimeric individual. The chimeric individual may be a pregnant subject and the heterogeneous sample may comprise a foreign molecule from a fetus and a molecule from the pregnant subject. In other instances, the chimeric individual is a recipient of a blood transfusion and the heterogeneous sample comprises a foreign molecule from a blood donor and a molecule from the recipient.

The sample can be obtained by a health care provider, for example, a physician, physician assistant, nurse, veterinarian, dermatologist, rheumatologist, dentist, paramedic, or surgeon. The sample can be obtained by a research technician. In some cases, information related to the sample such as the name of the subject, gender, ethnicity, national origin, race, disease-status, site where sample was obtained, name of person who obtained the sample, etc. is entered into a computer or database. In some cases, the information is transmitted to a different location.

In some instances, more than one sample from a subject is obtained. In some instances, at least 2, 3, 4, 5, 6, 7, 8, 9, 10, 11, 12, 13, 14, 15, 20, 25, or 30 samples are obtained from the subject. In some instances, the multiple samples are obtained over a period of time. For example, the multiple samples are obtained over a 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30-week period. Alternatively, the multiple samples are obtained over a 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30-month period. In some instances, the multiple samples are obtained over a 1, 1.5, 2, 2.5, 3, 3.5, 4, 4.5, 5, 6, 7, 8, 9, or 10-year period. In some instances, the multiple samples are obtained about every year, 6-months, 4-months, 3-months, 2-months, 1-month, 4-weeks, 3-weeks, 2-weeks, week, 4-days, 3-days, 2-days, day, 24-hours, 20-hours, 15-hours, 12-hours, 10-hours, 8-hours, 6-hours, 4-hours, 3-hours, 2-hours, or hour.

The biological sample may optionally be enriched for nucleic acid from one or more of the contributing genomic sources using techniques described herein. In the methods of the provided invention, the amount of RNA or DNA from a subject that can be analyzed includes, for example, as low as a single cell in some applications (e.g., a calibration test) and as many as 10 millions of cells or more translating to a range of DNA of 6 pg-60 ug, and RNA of approximately 1 pg-10 ug.

In some embodiments, less than 1 pg, 5 pg, 10 pg, 20 pg, 30 pg, 40 pg, 50 pg, 100 pg, 200 pg, 500 pg, 1 ng, 5 ng, 10 ng, 20 ng, 30 ng, 40 ng, 50 ng, 100 ng, 200 ng, 500 ng, 1 ug, 5 ug, 10 ug, 20 ug, 30 ug, 40 ug, 50 ug, 100 ug, 200 ug, 500 ug or 1 mg of nucleic acids are obtained from the heterogeneous biological sample for genotyping analysis. In some cases, about 1-5 pg, 5-10 pg, 10-100 pg, 100 pg-1 ng, 1-5 ng, 5-10 ng, 10-100 ng, 100 ng-1 ug of nucleic acids are obtained from the sample for genotyping analysis.

### VIII. Exemplary Methods

In some instances, the method comprises generally the steps of: (a) providing a heterogeneous sample from a subject in need thereof; (b) conducting a reaction on the heterogeneous sample to detect one or more foreign molecules; (c) optionally, diagnosing, predicting, or monitoring a status or outcome of a disease or condition based on the detection of one or more foreign molecules; and (d) optionally, determining a therapeutic regimen based on the detection of one or more foreign molecules.

In some instances, the method comprises (a) providing a biological sample containing genetic material from different genomic sources (e.g., a heterogeneous biological sample); (b) optionally, isolating one or more molecules (e.g., DNA, RNA or genomic DNA) from the heterogeneous biological sample; (c) optionally, amplifying the isolated nucleic acid molecule; (d) optionally, directly sequencing the isolated nucleic acid without diluting the genetic material within the sample or distributing the mixture of genetic material into discrete reaction samples; (e) optionally, counting the number of sequences for each genome in the heterogeneous sample; and (f) optionally, conducting an analysis that compares the ratios of the various unique sequences to determine relative amounts of the unique sequences and/or different genomes in the heterogeneous biological sample. Counting the number of sequences for each genome may be achieved via sequence reads. Alternatively, counting the number of sequences for each genome may comprise labeling the molecules. Labeling may comprise the use of barcodes. The method may further comprise mapping one or more unique sequences to one or more genomes represented within the sample. The methods may further comprise diagnosing a disease or condition, predicting the status or outcome of a disease or condition, monitoring the status or outcome of a disease or condition, differentially diagnosing the origin of a graft injury, or determining a therapeutic regimen. In some instances, the methods further comprise the use of a computer, computer software, and/or algorithm for analyzing one or more molecules in the sample. In other instances, the methods further comprise generating a report.

The reaction may comprise sequencing the foreign molecules. Alternatively, the reaction comprises hybridizing the foreign molecules to an array. In some instances, the reaction comprises quantifying the foreign molecules. Quantifying the foreign molecules may comprise determining the absolute amount of a foreign molecule. Quantifying the foreign molecules may comprise a comparative method or a non-comparative method. Quantifying the foreign molecules may comprise quantitative PCR. Alternatively, quantifying the foreign molecules may comprise labeling the foreign molecules with barcodes or labels. The reaction may comprise generating a size profile of the foreign molecules. In some instances, the reaction comprises amplifying the foreign molecules. Amplification may comprise a PCR-based method or a non-PCR based method. Alternatively, the reaction may comprise an amplification-free reaction. The reaction may comprise hybridizing the foreign molecules to a solid support. In some instances, the solid support is a microarray. In some instances, the solid support is a bead. The reaction may comprise a multiplex reaction. Alternatively, the reaction may comprise two or more sequential reactions.

In some instances, the disease or condition is organ transplant rejection. The disease or condition may be a cancer. Alternatively, the disease or condition is fetal genetic disorder. The disease or condition may also be pathogenic infection. Examples of pathogenic infections include, but are not limited to, bacterial infections, viral infections, fungal infections, and protozoan infections.

Determining a therapeutic regimen may comprise administering a therapeutic drug. Alternatively, determining a therapeutic regimen comprises modifying, continuing, or initiating a therapeutic regime. Alternatively, determining a therapeutic regimen comprises treating the disease or condition. In some instances, the therapy is an immunosuppressive therapy, anticancer therapy, or antimicrobial therapy. In other instances, diagnosing, predicting, or monitoring a disease or condition comprises determining the efficacy of a therapeutic regimen. Diagnosing, predicting, or monitoring a disease or condition comprises determining drug resistance. In some instances, monitoring a disease or condition comprises detecting transplant rejection. Predicting a disease or condition can comprise predicting the risk of a transplant rejection. Diagnosing a disease or condition may comprise diagnosing a fetal genetic disorder. In some instances, predicting a disease or condition comprises determining the risk of a fetal genetic disorder.

### IX. Performance

In some embodiments, the invention provides highly sensitive, non-invasive diagnostics for monitoring the health of a transplanted organ and managing the overall-health of the recipient using partial or whole genome analysis of circulating nucleic acids derived from tumors as compared to the patient's genome. Often, the methods of the invention deliver a higher detection rate of molecules (e.g., circulating nucleic acids, foreign molecules) in a host subject. For example, the methods of the invention may detect at least about 1.5-fold, 2-fold, 3-fold, 4-fold, 5-fold, 6-fold, 7-fold, or 10-fold more molecules than current methods.

The methods of the invention often provide improved clinical performance compared to conventional screening methods. In some instances, improved clinical performance comprises earlier diagnosis of a disease or condition. Alternatively, improved clinical performance comprises improved prediction of a status or outcome.

In some instances, the accuracy of the diagnosis, prediction, or monitoring a status or outcome of a disease or condition is at least about 50%, at least about 55%, at least about 60%, at least about 65%, at least about 70%, or at least about 75%. Preferably, the accuracy of the diagnosis, prediction, or monitoring of a disease or condition is at least about 80%, at least about 85%, at least about 90%, at least about 95%, or at least about 97%.

In some instances, the methods, compositions, and systems disclosed herein further comprise the use of a proportional-integral-derivative controller (PID controller). Generally, a PID controller is a generic control loop feedback mechanism (controller). A PID controller can calculate an “error” value as the difference between a measured process variable and a desired setpoint. The controller can attempt to minimize the error by adjusting the process control inputs. Generally, the PID controller calculation (algorithm) involves three separate constant parameters, and is accordingly sometimes called three-term control: the proportional, the integral and derivative values, denoted P, I, and D. These values can be interpreted in terms of time: P depends on the present error, I on the accumulation of past errors, and D is a prediction of future errors, based on current rate of change. The weighted sum of these three actions can be used to adjust the process via a control element.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

### EXAMPLES

### Example 1

### Differential Diagnosis of the Origin of a Graft Injury

In response to a rise in donor DNA levels, differential diagnosis of the origin of graft injury can be performed by looking at the size profile of donor DNA molecules identified in the cell-free fraction. Cell-free DNA is released from both apoptotic and necrotic cells, but the size distribution of DNAs differs in these two cases. Apoptotic cell death involves nuclease digestion of the genomic DNA while still bound to nucleosomes prior to release from the cell. Consequently, apoptotic contributions to the cell-free DNA are small fragments that form a ladder of sizes starting around 180 bp, then 360 bp, then 540 bp, and so on with the majority of molecules at the smallest sizes. Necrotic cell death is not as orderly and the released DNA is generally of a larger size, and is not digested into a smooth ladder of sizes but instead is a smear. Different causes of graft injury can give a different proportion of apoptotic and necrotic cell death. Infectious pathogens, for example, often express RNA messages that turn off cellular apoptotic processes, and increased necrotic contribution of cellular DNA is expected in such cases.

To assay the size distribution of donor DNA molecules, paired-end sequencing of cell-free DNA libraries made from fluids, including but not limited to plasma and urine, is performed. These libraries may be prepared with size selection, to assay smaller/larger ranges than normal, or without additional size selection after preparation. Following sequencing, the recipient/donor SNP approach is used to identify all molecules of donor origin, and the size of the inserts for this population calculated from the paired-end sequence alignment. A histogram of sizes can be made and features of this distribution, including mean/median size and the extent of apoptotic ladder-like patterning, is used to determine the ratio of apoptotic versus necrotic contribution. This ratio is used (possibly in combination with other signals including overall donor DNA levels, sequences from an infectious agent, or sequences from the immune repertoire) to perform the differential diagnosis of different causes of graft injury.

### Example 2

### Predicting Transplant Rejection

A blood sample from a transplant recipient is analyzed for donor-derived DNA in order to predict a risk of transplant rejection. The donor-derived cell-free DNA is detected via sequencing. Long-sequencing technology is used to sequence at least about 1500 bp of the donor-derived cell-free DNA. The amount of donor-derived cell-free DNA is quantified by counting the number of sequence reads. A transplant rejection is predicted if the total percentage of the donor-derived cell-free DNA is greater than about 1% of the total DNA in the sample.

### Example 3

### Modifying an Immunosuppressive Regimen

A urine sample from a liver transplant recipient treated with an immunosuppressive regimen is analyzed for donor-derived DNA in order to monitor an immunosuppressive regimen. A small fragment sample is generated by isolating DNA fragments less than about 150 base pairs from the urine sample. The amount of donor-derived DNA in the small fragment sample is determined by quantitative PCR. The amount of donor-derived DNA is less than about 0.5% of the total DNA in the small fragment sample, indicating that the donor graft is healthy. As a result of the healthy graft, one or more immunosuppressive drugs in the immunosuppressive regimen are reduced.

Alternatively, multiple urine samples are obtained from a heart transplant recipient treated with an immunosuppressive regimen are analyzed for kidney-derived DNA in order to monitor an immunosuppressive regimen. The urine samples are collected at different time points (e.g., 0 days, 7 days, 14 days, and 21 days after administration of an immunosuppressive regimen). For each urine sample at each time point, a large fragment sample is generated by isolating DNA fragments greater than about 150 base pairs from each urine sample. The amount of kidney-derived DNA in the large fragment sample is determined by digital PCR. The amount of kidney-derived DNA in the large fragment increases over time, indicating increased drug nephrotoxicity. As a result of the nephrotoxicity, one or more immunosuppressive therapies in the immunosuppressive regimen are reduced or removed from the regimen. Optionally, one or more immunosuppressive therapies in the immunosuppressive regimen are replaced with an alternative therapy.

### Example 4

### Determining an Anti-Cancer Regimen

A serum sample from a subject suffering from a breast cancer is analyzed for cancer cell-derived RNA. The nucleic acids in the sample are mixed with a plurality of unique barcodes to produce barcoded-RNA molecules. The barcoded-RNA molecules are reverse transcribed to produce barcoded-DNA molecules. The barcoded-DNA molecules are sequenced and the quantity of the nucleic acids is determined by counting the number of unique barcodes for each sequence. If the amount of the cancer cell-derived RNA increases by at least about 2-fold, then an anti-cancer regimen is administered or increased.

### Example 5

### Identification of Drug Responders

A urine sample from a subject suffering from a bacterial infection is analyzed for the presence of bacterial DNA. Multiple urine samples are collected from the subject suffering from the bacterial infection. The subject is concurrently treated with an antibiotic and the amount of bacterial DNA in the urine sample is detected over time. If the amount of bacterial DNA in the sample decreases over time, then the subject is identified as a responder to the antibiotic therapy.

### Example 6

### Determining an Antiviral Regimen

A serum sample from a subject suffering from a viral infection is analyzed for the presence of viral RNA. The viral RNA is isolated from the serum sample and reverse transcribed into DNA. The viral DNA is sequenced and the strain of the virus is determined. An antiviral regimen is administered based on the strain of the virus. Seven days after administration of the antiviral regimen, a second serum sample is obtained from the subject. A size profile of the subject-derived DNA is generated. The size profile indicates high necrotic cell death. The antiviral regimen is terminated and a new antiviral regimen is administered. Seven days after administration of the second antiviral regimen, a third serum sample is obtained from the subject. A size profile of the subject-derived DNA is generated. The size profile reveals normal levels of necrotic cell death. The second antiviral regimen is maintained until the viral infection is no longer detected.

1. A method comprising:
a. obtaining a sample from a subject who is the recipient of transplanted tissue;
b. inserting the sample into a device that generates a size profile of a set of molecules derived from the transplanted tissue; and
c. using the size profile to evaluate the level of necrosis in the transplanted tissue.

2-34. (canceled)

35. A method of treating a subject, the method comprising the steps of:
a) administering a therapeutic regimen to the subject;
b) obtaining a biological sample from the subject comprising circulating cell-free nucleic acids, wherein the circulating cell-free nucleic acids comprise nucleic acids from cancer tissue and normal tissue;
c) detecting an amount of the cell-free nucleic acids from cancer tissue in the biological sample; and
d) adjusting the therapeutic regimen administered to the subject based on the amount of the cell-free nucleic acids from the cancer tissue in the biological sample, wherein the therapeutic regimen is increased if the amount of the cell-free nucleic acids from the cancer tissue in the biological sample is greater than a threshold value.

36-38. (canceled)

39. A method of treating a subject, the method comprising the steps of:
a) administering a therapeutic regimen to the subject;
b) at a first point in time, obtaining a first biological sample comprising circulating cell-free nucleic acids from the subject, wherein the circulating cell-free nucleic acids comprise nucleic acids from normal tissue and cancer tissue;
c) detecting a first quantity of the cell-free nucleic acids from the cancer tissue in the biological sample;
d) at a second point in time, obtaining a second biological sample from the subject, wherein the second point of time is within a time period after the obtaining of the first biological sample of the subject;
e) detecting a second quantity of the cell-free nucleic acids from the cancer tissue in the second biological sample; and
f) adjusting the therapeutic regimen administered to the subject based on the first and second quantities, wherein the therapeutic regimen is increased if the second quantity of cell-free nucleic acids is greater than the first quantity of cell-free nucleic acids.

40-44. (canceled)

45. The method of claim 35, wherein the biological sample is blood.

46-49. (canceled)

50. The method of claim 35, wherein the cancer is prostrate cancer, breast cancer, ovarian cancer, lung cancer, colon cancer, pancreatic cancer, leukemia, lymphoma., central nervous system, or skin cancer.

51. (canceled)

52. The method of claim 35, wherein the therapeutic regimen is reduced by at least 50%.

53-56. (canceled)

57. The method of claim 35, wherein the therapeutic regimen is a chemotherapeutic regimen, a radiation therapy regimen, a monoclonal antibody regimen, an anti-angiogenic regimen, an oligonucleotide therapeutic regimen, or any combination thereof.

58. (canceled)

59. The method of claim 35, wherein the detecting further comprises conducting a sequencing reaction on the cell-free nucleic acids.

60. The method of claim 59, wherein the sequencing reaction is a next generation sequencing reaction.

61-62. (canceled)

63. The method of claim 35, wherein the detecting comprises detecting one or more of the following: a variable number tandem repeat (VNTR), a short tandem repeat (STR), a single nucleotide polymorphism (SNP) pattern, a hypervariable region, a dinucleotide repeat, a trinucleotide repeat, a tetranucleotide repeat, or a simple sequence repeat.

64-74. (canceled)

75. The method of claim 35, wherein the detecting comprises using an array.

76. The method of claim 75, wherein the array is a single nucleotide polymorphism array.

77. The method of claim 35, wherein the circulating cell-free nucleic acids comprise DNA.

78. The method of claim 35, wherein the detecting comprises detecting a mutation in an oncogene, a microsatellite alteration, or a viral genomic sequence.

79. The method of claim 35, wherein the detecting comprises detecting the presence of at least 25 genetic loci.

80. The method of claim 35, wherein the detecting discriminates and quantitates the expression of at least 25 genes.

81. The method of claim 35, wherein the detecting discriminates a DNA region containing a polymorphism.

82. The method of claim 35, further comprising inserting the biological sample into a device that generates a size profile of a set of molecules derived from the biological sample.

83. The method of claim 35, wherein the circulating cell-free nucleic acids comprise RNA.

84. The method of claim 35, wherein the detecting comprises detecting a copy number variation.

85. The method of claim 35, wherein the detecting comprises detecting a nucleic acid translocation or a nucleic acid rearrangement.

86. The method of claim 35, wherein the detecting comprises detecting a nucleic acid deletion.

87. The method of claim 50, wherein the lung cancer is selected from the group consisting of non-small cell lung carcinoma, small cell lung carcinoma, and mesothelioma.

88. The method of claim 50, wherein the leukemia is selected from the group consisting of acute lymphocytic leukemia, acute myelocytic leukemia, chronic lymphocytic leukemia, and chronic myelocytic leukemia.

89. The method of claim 50, wherein the lymphoma is a Hodgkin's lymphoma or a non-Hodgkin's lymphoma.

90. The method of claim 50, wherein the central nervous system cancer is a glioma or non-glioma.

91. The method of claim 39, wherein the biological sample is blood.

92. The method of claim 39, wherein the cancer is prostrate cancer, breast cancer, ovarian cancer, lung cancer, colon cancer, pancreatic cancer, leukemia, lymphoma, central nervous system, or skin cancer.

93. The method of claim 39, wherein the therapeutic regimen is reduced by at least 50%.

94. The method of claim 39, wherein the therapeutic regimen is a chemotherapeutic regimen, a radiation therapy regimen, a monoclonal antibody regimen, an anti-angiogenic regimen, an oligonucleotide therapeutic regimen, or any combination thereof.

95. The method of claim 39, wherein the detecting comprises conducting a sequencing reaction on the nucleic acids.

96. The method of claim 99, wherein the sequencing reaction is a next generation sequencing reaction.

97. The method of claim 39, wherein the detecting comprises using an array.

98. The method of claim 97, wherein the array is a single nucleotide polymorphism array.

99. The method of claim 39, wherein the circulating nucleic acids comprise DNA.

100. The method of claim 39, wherein the detecting comprises detecting a mutation in an oncogene, a microsatellite alteration, or a viral genomic sequence.

101. The method of claim 39, wherein the detecting comprises detecting the presence of at least 25 genetic loci.

102. The method of claim 39, wherein the detecting discriminates and quantitates the expression of at least 25 genes.

103. The method of claim 39, wherein the second point of time is within a three-month period after the obtaining of the first biological sample of the subject.

104. The method of claim 39, wherein the second point of time is within a six-month period after the obtaining of the first biological sample of the subject.

105. The method of claim 39, wherein the second point of time is within a two-year time period after the obtaining of the first biological sample of the subject.
